SEQUENCE LISTING (29 sequences)

SEQ ID NO: 1 - protein, 135 aa, Homo sapiens
ARTKQTARKSTGGKAPRKQLATKAARKSAPATGGVKKPHRYRPGTVALREIRRYQKSTELLIRKLPFQRLVREIAQDFKTDLRFQSSAVMALQEASEAYLVGLFEDTNLCAIHAKRVTIMPKDIQLARRIRGERA

SEQ ID NO: 2 - protein, 20 aa, Artificial
  N-terminal cleavage fragment H3
ARTKQTARKSTGGKAPRKQL

SEQ ID NO: 3 - protein, 114 aa, Artificial
  C-terminal cleavage fragment H3
TKAARKSAPATGGVKKPHRYRPGTVALREIRRYQKSTELLIRKLPFQRLVREIAQDFKTDLRFQSSAVMALQEASEAYLVGLFEDTNLCAIHAKRVTIMPKDIQLARRIRGERA

SEQ ID NO: 4 - protein, 6 aa, Artificial
  histone proteolysis motif
KQLATK

SEQ ID NO: 5 - protein, 10 aa, Artificial
  H3 peptide fragment
TKAARXSAPX

SEQ ID NO: 6 - protein, 8 aa, Artificial
  H3 peptide fragment
SAXATGGV

SEQ ID NO: 7 - DNA, 58 bp, Artificial
  siRNA to mouse Cathepsin L
ccggccagctatcctgtcgtgaattctcgagaattcacgacaggatagctggtttttg

SEQ ID NO: 8 - DNA, 57 bp, Artificial
  siRNA to human Cathepsin L1
ccgggaattgcctcagctactctaactcgagttagagtagctgaggcaattcttttt

SEQ ID NO: 9 - protein, 334 aa, Mus musculus
MNLLLLLAVLCLGTALATPKFDQTFSAEWHQWKSTHRRLYGTNEEEWRRAIWEKNMRMIQLHNGEYSNGQHGFSMEMNAFGDMTNEEFRQVVNGYRHQKHKKGRLFQEPLMLKIPKSVDWREKGCVTPVKNQGQCGSCWAFSASGCLEGQMFLKTGKLISLSEQNLVDCSHAQGNQGCNGGLMDFAFQYIKENGGLDSEESYPYEAKDGSCKYRAEFAVANDTGFVDIPQQEKALMKAVATVGPISVAMDASHPSLQFYSSGIYYEPNCSSKNLDHGVLLVGYGYEGTDSNKNKYWLVKNSWGSEWGMEGYIKIAKDRDNHCGLATAASYPVVN

SEQ ID NO: 10 - protein, 333 aa, Homo sapiens [sequence omitted]

SEQ ID NO: 11 - protein, 227 aa, Mus musculus
EPLMLKIPKSVDWREKGCVTPVKNQGQCGSCWAFSASGCLEGQMFLKTGKLISLSEQNLVDCSHAQGNQGCNGGLMDFAFQYIKENGGLDSEESYPYEAKDGSCKYRAEFAVANDTGFVDIPQQEKALMKAVATVGPISVAMDASHPSLQFYSSGIYYEPNCSSKNLDHGVLLVGYGYEGTDSNKNKYWLVKNSWGSEWGMEGYIKIAKDRDNHCGLATAASYPVVN

SEQ ID NO: 12 - protein, 226 aa, Homo sapiens
EPLFYEAPRSVDWREKGYVTPVKNQGQCGSCWAFSATGALEGQMFRKTGRLISLSEQNLVDCSGPQGNEGCNGGLMDYAFQYVQDNGGLDSEESYPYEATEESCKYNPKYSVANDTGFVDIPKQEKALMKAVATVGPISVAIDAGHESFLFYKEGIYFEPDCSSEDMDHGVLVVGYGFESTESDNNKYWLVKNSWGEEWGMGGYVKMAKDRRNHCGIASAASYPTV

SEQ ID NO: 13 - protein, 221 aa, Mus musculus
IPKSVDWREKGCVTPVKNQGQCGSCWAFSASGCLEGQMFLKTGKLISLSEQNLVDCSHAQGNQGCNGGLMDFAFQYIKENGGLDSEESYPYEAKDGSCKYRAEFAVANDTGFVDIPQQEKALMKAVATVGPISVAMDASHPSLQFYSSGIYYEPNCSSKNLDHGVLLVGYGYEGTDSNKNKYWLVKNSWGSEWGMEGYIKIAKDRDNHCGLATAASYPVVN

SEQ ID NO: 14 - protein, 220 aa, Homo sapiens
APRSVDWREKGYVTPVKNQGQCGSCWAFSATGALEGQMFRKTGRLISLSEQNLVDCSGPQGNEGCNGGLMDYAFQYVQDNGGLDSEESYPYEATEESCKYNPKYSVANDTGFVDIPKQEKALMKAVATVGPISVAIDAGHESFLFYKEGIYFEPDCSSEDMDHGVLVVGYGFESTESDNNKYWLVKNSWGEEWGMGGYVKMAKDRRNHCGIASAASYPTV

SEQ ID NO: 15 - DNA, 1005 bp, Mus musculus
atgaatcttttactccttttggctgtcctctgcttgggaacagccttagctactccaaaatttgatcaaacctttagtgcagagtggcaccagtggaagtccacacacagaagactgtatggcacgaatgaggaagagtggaggagagcgatatgggagaagaacatgagaatgatccagctacacaacggggaatacagcaacgggcagcacggcttttccatggagatgaacgccttcggtgacatgaccaatgaggaattcaggcaggtggtgaatggctaccgccaccagaagcacaagaaggggaggctttttcaggaaccgctgatgcttaagatccccaagtctgtggactggagagaaaagggttgtgtgactcctgtgaagaaccagggccagtgcgggtcttgttgggcgtttagcgcatcgggttgcctagaaggacagatgttccttaagaccggcaaactgatctcactgagtgaacagaaccttgtggactgttctcacgctcaaggcaatcagggctgtaacggaggcctgatggattttgctttccagtacattaaggaaaatggaggtctggactcggaggagtcttacccctatgaagcgaaggacggatcttgtaaatacagagccgagttcgctgtggctaatgacacagggttcgtggatatccctcagcaagagaaagccctcatgaaggctgtggcgactgtggggcctatttctgttgctatggacgcaagccatccgtctctccagttctatagttcaggcatctactatgaacccaactgtagcagcaagaacctcgaccatggggttctgttggtgggctatggctatgaaggaacagattcaaataagaataaatattggcttgtcaagaacagctggggaagtgaatggggtatggaaggctacatcaaaatagccaaagaccgggacaaccactgtggacttgccaccgcggccagctatcctgtcgtgaattga

SEQ ID NO: 16 - DNA, 1002 bp, Homo sapiens
atgaatcctacactcatccttgctgccttttgcctgggaattgcctcagctactctaacatttgatcacagtttagaggcacagtggaccaagtggaaggcgatgcacaacagattatacggcatgaatgaagaaggatggaggagagcagtgtgggagaagaacatgaagatgattgaactgcacaatcaggaatacagggaagggaaacacagcttcacaatggccatgaacgcctttggagacatgaccagtgaagaattcaggcaggtgatgaatggctttcaaaaccgtaagcccaggaaggggaaagtgttccaggaacctctgttttatgaggcccccagatctgtggattggagagagaaaggctacgtgactcctgtgaagaatcagggtcagtgtggttcttgttgggcttttagtgctactggtgctcttgaaggacagatgttccggaaaactgggaggcttatctcactgagtgagcagaatctggtagactgctctgggcctcaaggcaatgaaggctgcaatggtggcctaatggattatgctttccagtatgttcaggataatggaggcctggactctgaggaatcctatccatatgaggcaacagaagaatcctgtaagtacaatcccaagtattctgttgctaatgacaccggctttgtggacatccctaagcaggagaaggccctgatgaaggcagttgcaactgtggggcccatttctgttgctattgatgcaggtcatgagtccttcctgttctataaagaaggcatttattttgagccagactgtagcagtgaagacatggatcatggtgtgctggtggttggctacggatttgaaagcacagaatcagataacaataaatattggctggtgaagaacagctggggtgaagaatggggcatgggtggctacgtaaagatggccaaagaccggagaaaccattgtggaattgcctcagcagccagctaccccactgtgtga

SEQ ID NO: 17 - DNA, 684 bp, Mus musculus
gaaccgctgatgcttaagatccccaagtctgtggactggagagaaaagggttgtgtgactcctgtgaagaaccagggccagtgcgggtcttgttgggcgtttagcgcatcgggttgcctagaaggacagatgttccttaagaccggcaaactgatctcactgagtgaacagaaccttgtggactgttctcacgctcaaggcaatcagggctgtaacggaggcctgatggattttgctttccagtacattaaggaaaatggaggtctggactcggaggagtcttacccctatgaagcgaaggacggatcttgtaaatacagagccgagttcgctgtggctaatgacacagggttcgtggatatccctcagcaagagaaagccctcatgaaggctgtggcgactgtggggcctatttctgttgctatggacgcaagccatccgtctctccagttctatagttcaggcatctactatgaacccaactgtagcagcaagaacctcgaccatggggttctgttggtgggctatggctatgaaggaacagattcaaataagaataaatattggcttgtcaagaacagctggggaagtgaatggggtatggaaggctacatcaaaatagccaaagaccgggacaaccactgtggacttgccaccgcggccagctatcctgtcgtgaattga

SEQ ID NO: 18 - DNA, 681 bp, Homo sapiens
gaacctctgttttatgaggcccccagatctgtggattggagagagaaaggctacgtgactcctgtgaagaatcagggtcagtgtggttcttgttgggcttttagtgctactggtgctcttgaaggacagatgttccggaaaactgggaggcttatctcactgagtgagcagaatctggtagactgctctgggcctcaaggcaatgaaggctgcaatggtggcctaatggattatgctttccagtatgttcaggataatggaggcctggactctgaggaatcctatccatatgaggcaacagaagaatcctgtaagtacaatcccaagtattctgttgctaatgacaccggctttgtggacatccctaagcaggagaaggccctgatgaaggcagttgcaactgtggggcccatttctgttgctattgatgcaggtcatgagtccttcctgttctataaagaaggcatttattttgagccagactgtagcagtgaagacatggatcatggtgtgctggtggttggctacggatttgaaagcacagaatcagataacaataaatattggctggtgaagaacagctggggtgaagaatggggcatgggtggctacgtaaagatggccaaagaccggagaaaccattgtggaattgcctcagcagccagctaccccactgtgtga

SEQ ID NO: 19 - DNA, 666 bp, Mus musculus
atccccaagtctgtggactggagagaaaagggttgtgtgactcctgtgaagaaccagggccagtgcgggtcttgttgggcgtttagcgcatcgggttgcctagaaggacagatgttccttaagaccggcaaactgatctcactgagtgaacagaaccttgtggactgttctcacgctcaaggcaatcagggctgtaacggaggcctgatggattttgctttccagtacattaaggaaaatggaggtctggactcggaggagtcttacccctatgaagcgaaggacggatcttgtaaatacagagccgagttcgctgtggctaatgacacagggttcgtggatatccctcagcaagagaaagccctcatgaaggctgtggcgactgtggggcctatttctgttgctatggacgcaagccatccgtctctccagttctatagttcaggcatctactatgaacccaactgtagcagcaagaacctcgaccatggggttctgttggtgggctatggctatgaaggaacagattcaaataagaataaatattggcttgtcaagaacagctggggaagtgaatggggtatggaaggctacatcaaaatagccaaagaccgggacaaccactgtggacttgccaccgcggccagctatcctgtcgtgaattga

SEQ ID NO: 20 - DNA, 663 bp, Homo sapiens
gcccccagatctgtggattggagagagaaaggctacgtgactcctgtgaagaatcagggtcagtgtggttcttgttgggcttttagtgctactggtgctcttgaaggacagatgttccggaaaactgggaggcttatctcactgagtgagcagaatctggtagactgctctgggcctcaaggcaatgaaggctgcaatggtggcctaatggattatgctttccagtatgttcaggataatggaggcctggactctgaggaatcctatccatatgaggcaacagaagaatcctgtaagtacaatcccaagtattctgttgctaatgacaccggctttgtggacatccctaagcaggagaaggccctgatgaaggcagttgcaactgtggggcccatttctgttgctattgatgcaggtcatgagtccttcctgttctataaagaaggcatttattttgagccagactgtagcagtgaagacatggatcatggtgtgctggtggttggctacggatttgaaagcacagaatcagataacaataaatattggctggtgaagaacagctggggtgaagaatggggcatgggtggctacgtaaagatggccaaagaccggagaaaccattgtggaattgcctcagcagccagctaccccactgtgtga

SEQ ID NO: 21 - protein, 5 aa, Artificial
  pentapeptide amide inhibitor of cathepsin-L
LLLTR

SEQ ID NO: 22 - protein, 5 aa, Artificial
  pentapeptide amide inhibitor of cathepsin-L
RKLLW

SEQ ID NO: 23 - protein, 5 aa, Artificial
  pentapeptide amide inhibitor of cathepsin-L
LFLTR

SEQ ID NO: 24 - protein, 5 aa, Artificial
  pentapeptide amide inhibitor of cathepsin-L
RKLWL

SEQ ID NO: 25 - protein, 5 aa, Artificial
  pentapeptide amide inhibitor of cathepsin-L
RKLWD

SEQ ID NO: 26 - protein, 16 aa, Artificial
  Cathepsin L peptide
ENGGLDSEESYPYEAK

SEQ ID NO: 27 - protein, 14 aa, Artificial
  Cathepsin L peptide
NSWGSEWGMEGYIK

SEQ ID NO: 28 - protein, 18 aa, Artificial
  Cathepsin L peptide
DRDNHCGLATAASYPVVN

SEQ ID NO: 29 - protein, 16 aa, Artificial
  Cathepsin L peptide
DNHCGLATAASYPVVN

(19) United States
(12) Patent Application Publication
(10) Pub. No.: US 2011/0312897 A1
(43) Pub. Date: Dec. 22, 2011

(54) CATHEPSIN L PROTEOLYTICALLY PROCESSES HISTONE H3 DURING MOUSE EMBRYONIC STEM CELL DIFFERENTIATION

(75) Inventors: C. David Allis, Princeton, NJ (US); Elizabeth Duncan, Prairie Village, KS (US)

(73) Assignee: THE ROCKEFELLER UNIVERSITY, New York, NY (US)

(21) Appl. No.: 13/119,503

(22) PCT Filed: Sep. 17, 2009
(86) PCT No.: PCT/US2009/057249

§ 371 Date: Jul. 11, 2011

Related U.S. Application Data
(60) Provisional application No. 61/097,679, filed on Sep. 17, 2008.

(51) Int. Cl. A61K 38/00; A61P 35/00; A61K 31/7105; C12N 5/00; C12Q 1/37

(52) U.S. Cl. 514/19.3; 435/377; 435/375; 435/24; 514/44 A

(57) ABSTRACT

Methods and agents useful for modulating histone proteolysis, stem cell differentiation, and gene transcription and for treating cancer are disclosed. Antibodies or antigen binding fragments that selectively bind to histone-3 cleavage products and are useful for diagnosing cancer and monitoring a subject's response to cancer treatment are also disclosed.

This application claims the benefit of U.S. Provisional Patent Application Ser. No. 61/097,697, filed Sep. 17, 2008, which is hereby incorporated in its entirety.

The subject matter of this application was made with support from the United States Government under the National Institutes of Health, Grant No. RO1-GM53512. The government has certain rights in this invention.

### FIELD OF THE INVENTION

The present invention describes methods and agents useful for modulating histone proteolysis, stem cell differentiation, and gene transcription. The invention further describes methods and agents for diagnosing and treating cancer based on histone proteolysis.

### BACKGROUND OF THE INVENTION

Embryonic stem cells (ESCs) undergo dramatic changes in morphology, cell cycle, and gene expression as they differentiate into defined cell types (Kim et al., “An Extended Transcriptional Network for Pluripotency of Embryonic Stem Cells,” Cell 132:1049-1061 (2008) and Murry et al., “Differentiation of Embryonic Stem Cells to Clinically Relevant Populations: Lessons from Embryonic Development,” Cell 132:661-680 (2008)). An increasing body of literature demonstrates that these changes extend to, if not originate from, changes in genomic and epigenomic organization, which together enable cells to establish and maintain cellular identity. Since, in vivo, eukaryotic genomes are intimately associated with histone proteins to form chromatin, this physiologically-relevant structure must be remodeled as part of a large-scale mechanism to achieve rapid and drastic changes in gene expression (Amey et al., “Epigenetic Aspects of Differentiation,” J Cell Sci 117:4355-4363 (2004)). Undifferentiated cells, for example, typically display increased physical plasticity and less compacted chromatin than their differentiated counterparts (Meshorer et al., “Hyperdynamic Plasticity of Chromatin Proteins in Pluripotent Embryonic Stem Cells,” Dev Cell 10:105-116 (2006) and Pajerowski et al., “Physical Plasticity of the Nucleus in Stem Cell Differentiation,” Proc Natl Acad Sci USA 104:15619-15624 (2007)). On a molecular level, undifferentiated cells undergo radical changes in gene expression as they differentiate; conveniently providing markers of “stemness” whose expression dramatically decreases (e.g., the transcription factor Oct 3/4) as differentiation progresses. Such evidence of change on both the cell biological and molecular levels suggests that cells undergo a significant reorganization of their genome during the differentiation process and that, moreover, this transition must be carefully regulated in order for the cell to differentiate properly and adopt a specific lineage.

Recent studies have shown that histone covalent modification patterns change significantly upon ESC differentiation (Giadrossi et al., “Chromatin Organization and Differentiation in Embryonic Stem Cell Models,” Curr Opin Genet Dev 17:132-138 (2007)). For example, core histones (H2A, H2B, H3 and H4) are largely deacetylated upon differentiation and histone deacetylase activity may be required for ESC differentiation (Lee et al., “Histone Deacetylase Activity is Required for Embryonic Stem Cell Differentiation,” Genesis 38:32-38 (2004)). Chromatin-immunoprecipitation (ChIP) experiments have also identified specific genes and/or genomic regions that change their “epigenetic signature” upon differentiation (Azuara et al., “Chromatin Signatures of Pluripotent Cell Lines,” Nat Cell Biol 8:532-538 (2006) and Bernstein et al., “A Bivalent Chromatin Structure Marks Key Developmental Genes in Embryonic Stem Cells,” Cell 125:315-326 (2006)).

Despite a wealth of emerging data describing changing patterns of epigenetic signatures during ESC differentiation, very little is known about the mechanisms used to achieve such change. Several possible mechanisms for removing the more stable histone modifications such as lysine methylation have been suggested and include: enzymatic demethylation, histone replacement, and regulated histone proteolysis (Bannister et al., “Histone Methylation: Recognizing the Methyl Mark,” Methods Enzymol 376:269-288 (2004)). Enzymatic activities have been identified that carry out the first two mechanisms (Ahmad et al., “The Histone Variant H3.3 Marks Active Chromatin by Replication-Independent Nucleosome Assembly,” Mol Cell 9:1191-1200 (2002) and Shi et al., “Histone Demethylation Mediated by the Nuclear Amine Oxidase Homolog LSD1,” Cell 119:941-953 (2004)) and there is precedence for controlled histone H3-specific proteolysis (Allis et al., “Proteolytic Processing of Histone H3 in Chromatin: A Physiologically Regulated Event in Tetrahymena Micronuclei,” Cell 20:55-64 (1980) and Falk et al., “Foot-and-Mouth Disease Virus Protease 3C Induces Specific Proteolytic Cleavage of Host Cell Histone H3,” J Virol 64:748-756 (1990)). However, specific, regulated, endogenous proteolysis has not been well documented in mammalian cells.

### SUMMARY OF THE INVENTION

A first aspect of the invention is directed to a method of administering to a cell an agent that modulates histone proteolysis at a motif comprising KQLATK (SEQ ID NO:4) of the histone.

A second aspect of the present invention is directed to a method of regulating stem cell differentiation. This method involves administering to a population of stem cells an agent that modulates histone proteolysis under conditions effective to regulate stem cell differentiation.

A third aspect of the present invention relates to a method of modulating gene transcription in a cell. This method involves administering to a population of cells an agent that modulates histone proteolysis under conditions effective to modulate gene transcription in the cell.

The present invention is also directed an antibody or antigen-binding fragment thereof that selectively binds to a histone-3 cleavage product.

Another aspect of the present invention relates to a method of diagnosing cancer in a subject, which involves providing a sample from the subject and contacting the sample with an antibody that selectively binds to a histone-3 cleavage product. The method further involves identifying the presence of a histone-3 cleavage product in the sample with the antibody that selectively binds to a histone-3 cleavage product and diagnosing cancer in the subject based on the identifying step.

Another aspect of the present invention relates to a method of monitoring a subject's response to cancer treatment. This method involves obtaining a first biological sample from the subject before administration of the cancer treatment and a second biological sample from the subject after administration of the cancer treatment and contacting the samples with an antibody that selectively binds to a histone-3 cleavage product. The presence of a histone-3 cleavage product in the samples is identified with the antibody and the subject's response to cancer treatment is monitored based on the presence or absence of the histone-3 cleavage product.

The present invention is also directed to a method of identifying candidate compounds useful for modulating histone proteolysis. This method involves providing the candidate compound and a population of differentiating stem cells, and contacting the candidate compound and the population of differentiating stem cells under conditions effective for the candidate compound to modulate histone proteolysis. The presence or absence of a histone cleavage product in the population of differentiated stem cells is detected and a compound useful for modulating histone proteolysis is identified based the presence or absence of a cleavage product.

An additional aspect of the present invention relates to a method of treating a subject having cancer. This method involves selecting a patient based on his/her propensity to undergo histone proteolysis at a motif comprising KQLATK (SEQ ID NO:4) and administering an agent that modulates histone proteolysis to the subject under conditions effective to treat cancer.

Another aspect of the present invention is directed to a method of administering to a cell an agent that inhibits histone proteolysis in the cell. The agent administered to the cell is a cathepsin inhibitor selected from the group consisting of a nucleic acid, a peptide, or a small molecule cathepsin inhibitor.

Another aspect of the present invention is directed to a method of administering to a cell an agent that induces histone proteolysis in the cell. The agent administered to the cell is a recombinant cathepsin protein or proteolytic active cathepsin polypeptide, or a nucleic acid molecule encoding the recombinant cathepsin protein or proteolytic active cathepsin polypeptide.

ESCs employ a novel, regulated histone proteolysis mechanism in order to change their “epigenetic signature” upon differentiation. Cathepsin L has been identified as a developmentally-regulated histone H3 protease whose activity may be modulated by the modification of the histone tail itself. Taken together, these studies call attention to novel nuclear functions of this family of cysteine proteases in histone and stem cell biology and suggest that controlled histone proteolysis may be part of a more general mechanism for introducing physiologically-relevant variation into the chromatin polymer than was previously known.

### BRIEF DESCRIPTION OF THE DRAWINGS

FIGS. 1A-1C show the distinct histone H3 species that were detected in chromatin during ESC differentiation. In FIG. 1A, undifferentiated (und) ESCs were differentiated with retinoic acid (RA) in a monolayer and harvested for whole cell extracts (WCEs) at the time points indicated. WCEs for each time point were analyzed by immunoblotting with the antibodies indicated to the right of each panel (H3gen refers to the H3 general C-terminal antibody). Molecular weights (in kD) are indicated to the left in this and all subsequent gels and immunoblots. In FIG. 1B, chromatin isolated from either undifferentiated ESCs (top panel) or those differentiated with RA for 3 days was subsequently digested with micrococcal nuclease for the indicated times. The solubilized chromatin pellet input (P), Mnase digested chromatin, and solubilized post-Mnase pellet (P′) was then analyzed by immunoblotting with an H3-general antibody. FIG. 1C shows ESCs that were differentiated using three basic methods: monolayer differentiation with RA (left), monolayer differentiation with leukemia inhibitory factor (LIF) withdrawal (middle), and embryoid body (EB) formation by cell aggregation (right). WCEs were analyzed as in FIG. 1A for both a marker of pluripotency (Oct 3/4, top panels) and the histone H3 sub-band.

FIGS. 2A-2C demonstrate histone H3 N-terminal cleavage during ESC differentiation. Histones were acid-extracted from 3 days+RA differentiating nuclei and purified using RP-HPLC. Fractions containing the histone H3 sub-band were pooled, re-fractionated by RP-HPLC, and the subsequent fractions were then screened by immunoblotting with an H3-general antibody as shown in FIG. 2A (left). Equal amounts of fractions 52-55 were pooled, separated by SDS-PAGE, transferred to PVDF membrane and stained with Ponceau Red (right). Both bands of the sub-band doublet (asterisks) were excised from the membrane and subjected to Edman degradation, which provided evidence that the faster migrating H3 had been N-terminally cleaved between residues A21 and T22, generating the peptide fragment of TKAARXSAPX (SEQ ID NO:5), and cleaved between residues K27 and S28 (right), generating the peptide fragment of SAXATGGV (SEQ ID NO:6). Residues not clearly identified in the generated sequence are denoted “X.” In FIG. 2B, the sample in fraction 54 from the RP-HPLC enrichment shown in FIG. 2A was digested with GluC, which generates intact N-terminal peptides that terminate with E50 (i.e., residues A1-E50 of SEQ ID NO:1). Peptides generated were then analyzed on a linear ion trap—Fourier transform mass spectrometer. The six highly modified, truncated peptide fragments of the GluC-generated 1-50 peptide that were observed are listed in FIG. 2B, right column, and include peptides consisting of residues T22-E50 of SEQ ID NO:1, residues K23-E50 of SEQ ID NO:1, residues A24-E50 of SEQ ID NO:1, residues A25-E50 of SEQ ID NO:1, residues K27-E50 of SEQ ID NO:1, and residues S28-E50 of SEQ ID NO:1. The detected post-translational modifications for each peptide fragment are summarized in FIG. 9. Summed ion currents for all charge states and modified forms of the above sequences were employed to estimate the relative abundances of the six peptides beginning with the indicated residues as follows: T22 and A24>than K23 and K27>A25 and S28 (FIG. 2B, C-terminal peptides). The peptide beginning with R26 was not detected. Note that the two sequences detected by Edman degradation were also detected by mass spectrometry (MS) (denoted by asterisks). All six of the truncated 1-50 peptides contain Ala at position 31 (underlined) and are thus derived from the H3 isoform H3.2. Three highly modified forms of the complementary N-terminal fragments consisting of residues A1-A21 of SEQ ID NO:1, residues A1-K23 of SEQ ID NO:1, and residues A1-R26 of SEQ ID NO:1 (FIG. 2B, left column) are also present in the same HPLC fraction. FIG. 2C shows the sequence of the mammalian histone H3 tail (residues 1-38 of SEQ ID NO:1) surrounding the cleavage sites mapped in FIGS. 2A and B. The bold solid line indicates the “primary” cleavage site mapped by both Edman degradation and MS (H3.cs1); additional significant cleavage sites are marked with regular solid lines; less abundant sites are marked by dashed lines. Lysines found by MS to be highly acetylated (ac) or methylated (me) are marked by a triangle or circle, respectively (see FIG. 9 for details).

FIGS. 3A-3E show detection of the cysteine protease Cathepsin L in fractions enriched for histone H3 cleavage activity. FIG. 3A is a schematic of the in vitro H3 cleavage assay. FIG. 3B is a representative example of the H3 cleavage assay comparing soluble cytosolic plus nuclear protein extract (S) and solubilized chromatin extract (C) from undifferentiated and differentiated ESCs (i.e., those cultured for 3 days+RA). FIG. 3C is a schematic of extract fractionation for protease enrichment. FIG. 3D shows an H3 cleavage assay of hydroxyapatite fractions generated by the scheme shown in FIG. 3C. Assay reactions were analyzed by immunoblotting with both HIS-HRP and H3.cs1 antibodies. In FIG. 3E, hydroxyapatite fractions assayed in FIG. 3D were analyzed for the presence of Cathepsin L by immunoblotting with Cathepsin L antibody; # designates proprotein (˜37 kD), • indicates intermediate processed form (˜30 kD), and * indicates mature processed form (˜25 kD) of the Cathepsin L protein.

FIGS. 4A-4D show Cathepsin L mediated cleavage of histone H3 in vitro and association with chromatin in vivo. FIG. 4A shows hydroxyapatite fraction #23 (FIG. 3D) assayed +/−protease inhibitors in the H3 cleavage assay. Cysteine protease inhibitor E64 is a potent inhibitor of the H3 protease activity in fraction #23. In FIG. 4B, immobilized E64 was incubated with both an active hydroxyapatite fraction, fraction 23, and a fraction without enzymatic activity, fraction 20; control resin was incubated with each fraction in parallel. Resins were precipitated from solution, boiled in SDS sample buffer to remove bound proteins, and analyzed by immunoblotting (FIG. 4B, bottom panel). The supernatant was tested for H3 protease activity (FIG. 4B, top panel). In FIG. 4C, hydroxyapatite fraction #23 (FIG. 3D) was assayed with various rH3-HIS point mutants in the H3 cleavage assay. Mutations in L20 abolished activity as assayed by both the HIS and H3.cs1 antibodies (top two panels) and mutations in K23 abolished cleavage as assayed by the H3.cs1 antibody (bottom panel). FIG. 4D shows chromatin from undifferentiated ESCs and ESCs differentiated +RA, for the number of days indicated, digested with micrococcal nuclease and analyzed by immunoblotting with Cathepsin L antibody to assay whether Cathepsin L protein is associated with chromatin. Note that Cathepsin L is associated with chromatin in differentiating ESCs, particularly the mature form (*).

FIGS. 5A-5C illustrate recombinant Cathepsin L (rCathepsin L) cleavage of histone H3 in vitro. Recombinant Cathepsin L cleaves rH3 in vitro at both pH 5.5 and pH 7.4 and generates a fragment that is recognized by both α-HIS-HRP (FIG. 5A, top panel) and α-H3.cs1 (FIG. 5A, bottom panel) antibodies. FIG. 5B shows a mass spectrometry analysis of recombinant mouse Cathepsin L cleavage products. rCathepsin L was incubated with recombinant H3-HIS at both pH 5.5 and pH 7.4; after 2 hours, the reaction products were subjected to analysis by mass spectrometry. Both N-terminal (left) and C-terminal (right) fragments of the rH3 cleavage were detected; note the similarity to the pattern of in vivo cleavage shown in FIG. 2B. The C-terminal peptides generated by Cathepsin L cleavage of H3 (FIG. 5B, right column) consist of residues T22-E50 of SEQ ID NO:1, residues K23-E50 of SEQ ID NO:1, residues A24-E50 of SEQ ID NO:1, residues A25-E50 of SEQ ID NO:1, residues K27-E50 of SEQ ID NO:1, residues S28-E50 of SEQ ID NO:1, and residues K37-E50 of SEQ ID NO:1. Summed ion currents for all charge states of the unmodified sequences were employed to estimate the relative abundances of the six peptides beginning with the indicated residues as follows: T22>K23, A24, and S28>A25 and K27. Also observed in both of the above digests are five of the six complementary N-terminal peptides generated by Cathepsin L cleavage of H3 (5B, left column) containing residues A1-A21 of SEQ ID NO:1, residues A1-T22 of SEQ ID NO:1, residues A1-K23 of SEQ ID NO:1, residues A1-R26 of SEQ ID NO:1, and residues A1-K27 of SEQ ID NO:1. Relative abundances of these N-terminal peptides correlate, as expected, with their C-terminal counterparts: A1-A21>A1-T22, A1-K23, A1-K27>A1-R26. FIG. 5C is an immunoblot of rH3-HIS cleavage following incubation with recombinant pre-activated Cathepsins B, K, and L at both pH 5.5 and pH 7.4 for 15 minutes.

FIGS. 6A-6C show the reduction in histone H3 cleavage by RNAi knockdown and chemical inhibition of Cathepsin L in vivo. Control and Ctsl RNAi cells lines were differentiated with RA as usual and harvested at the indicated time points. WCEs were then separated by SDS-PAGE and analyzed for both Cathepsin L expression (FIG. 6A, upper panel) and histone H3 cleavage (FIG. 6A, lower panel) by immunoblotting. FIG. 6B is an immunoblot analysis of samples from day 3 post-induction with RA serially diluted two-fold. The addition of Cathepsin L Inhibitor Ito the cell media of differentiating ESCs (FIG. 6C, panel a, left) inhibits the processing of Cathepsin L itself (panel a) as well as that of histone H3 (panels b, c) as compared to DMSO alone treated control cells (FIG. 6C, panel a, right). Loss of pluripotency marker Oct 3/4 was not affected (panel d) nor was the processing of another cathepsin family member, Cathepsin B (panel e).

FIGS. 7A-7D demonstrate that covalent histone modifications modulate Cathepsin L activity and its downstream effects. FIG. 7A is an immunoblot showing cleavage of the modified recombinant histone 3 substrates following incubation with rCathepsin L. The four rH3 substrates used in the cleavage assay include: 1=rH3 unmodified, 2=rH3 alkylated to K27me2, 3=rH3 pan-acetylated with acetic anhydride, 4=rH3+K27me2 pan-acetylated with acetic anhydride. FIG. 7B is a graph showing the amount of recombinant H3 cleavage product formed following incubation with Cathepsin L. H3 cleavage reactions were performed as in FIG. 7A using synthesized H3 peptides representing amino acids 15 to 31. Reactions were incubated with ˜250 pmol peptide and quenched with 0.1% TFA before being plated in duplicate for ELISA with the H3cs.1 antibody. Signal was normalized to that of mock reactions for each peptide. Results represent three independent experiments. FIG. 7C shows the results of peptide pull-down assays performed using the chromodomain of mouse CBX7 and the recombinant PHD finger of human BPTF (sequences and methylation status indicated). Peptide-bound polypeptides were resolved by SDS PAGE and visualized by silver staining FIG. 7D shows fluorescence anisotropy of Cbx7-CD protein binding to non-cleaved peptide (amino acid residues 18-37) vs. cleaved peptide (amino acid residues 22-37). Binding decreases 3-fold with cleaved peptide. p<0.01. K_{d}s are in μM±SEM. Data points represent the mean±SD

FIGS. 8A-8B show the faster migrating histone H3 sub-species appears to be lacking its amino terminus but is not cleaved during whole-cell extract preparation. In FIG. 8A, recombinant (r) histone H3, rH2A, H3 1-20 peptide, and RP-HPLC purified endogenous (e) H3 from differentiated ESCs (eH3 diff) were separated by SDS-PAGE and analyzed by immunoblotting with the H3-general antibody. Although the antibody generated against the H3C-terminus recognized the faster migrating H3 sub-band, the antibody generated against the H3N-terminus does not. To test whether uncontrolled proteolysis occurred upon cell lysis, unmodified rH3-HIS was added to SDS-Laemmli sample buffer prior to adding it to cell pellets and solubilization to generate WCEs (FIG. 8B). The unmodified rH3-HIS showed no evidence of cleavage when added to the lysing cells (FIG. 8B, right); however, endogenous H3 cleavage was detected in the lysates of 2 and 3 day differentiated ESC, as expected, which was shown by immunoblotting with an H3K27me2 antibody (FIG. 8B, left).

FIG. 9 is a summary of the post-translational modifications detected on proteolytically cleaved H3 obtained from differentiating ESCs. Material from fraction 54 (FIG. 2A) was digested with GluC to produce N-terminal, H3 fragments ending in E50. The resulting mixture was then analyzed by nano-flo HPLC interfaced with both a linear ion trap-Fourier transform mass spectrometer and a linear ion trap instrument equipped for electron transfer dissociation. Spectra recorded with the former instrument detected six, highly-modified truncated, N-terminal, histone H3-peptides consisting of residues T22-E50 of SEQ ID NO:1, residues K23-E50 of SEQ ID NO:1, residues A24-E50 of SEQ ID NO:1, residues A25-E50 of SEQ ID NO:1, residues K27-E50 of SEQ ID NO:1, and residues S28-E50 of SEQ ID NO:1 (FIG. 2B, right column). Post-translational modification detected by recording electron transfer dissociation (ETD) mass spectra on different isoforms of these six peptides are shown for each residue in the right panel of FIG. 9. Sixty-seven different forms of the above six peptides were characterized. Ten contain marks associated with active transcription (H3K23Ac, H3K36me, H3K36me2 or H3K36me3), ten contain marks associated with gene silencing (H3K27me, H3K27me2 or H3K27me3), and 43 contain combinations of above active and repressive marks. Three of the most abundant, complementary, N-terminal fragments generated by proteolytic cleavage of H3, (residues A1-A21 of SEQ ID NO:1, residues A1-K23 of SEQ ID NO:1, and residues A1-R26 of SEQ ID NO:1) were also detected in HPLC fraction 54 (FIG. 2B, left column). Post-translational modifications detected on different isoforms of these three peptides are shown in the left panel of FIG. 9. Monomethyl-, dimethyl, and trimethyl-marks are depicted by one, two, and three solid circles, respectively. Acetyl marks are depicted by triangles. An asterisk indicates that the modified isoform was detected by accurate mass measurement only. All ETD spectra were interpreted manually.

FIG. 10 shows that high-salt extraction of nuclei does not efficiently extract H3 cleavage activity. Nuclear extracts were prepared as described (Dignam et al., “Accurate Transcription Initiation by RNA Polymerase II in a Soluble Extract From Isolated Mammalian Nuclei,” Nucleic Acids Res 11:1475-1489 (1983), which is hereby incorporated by reference in it entirety) from 3 days+RA differentiated cells. After the initial extraction of nuclear proteins with 420 mM KCl (420), the chromatin pellet (P) was further extracted by sequential 60 mM increases in KCl concentration. The remaining chromatin pellet was then solubilized by sonication in buffer A. The cytosolic (cyt), high salt, and chromatin extracts were then assayed for H3 cleavage activity using the H3 cleavage assay described in FIG. 3.

FIGS. 11A-11C depict the characterization of the H3.cs1 antibody. FIG. 11A shows the 2× branched peptide sequence used to generate the antibody. FIG. 11B is an immunoblot showing the biological specificity of the anti-H3.cs1 antibody. Rabbit serum containing the H3.cs1 antibody was used to immunoblot WCE samples from undifferentiated, 3 days+RA, and 5-days+RA. In FIG. 11C, rabbit serum was tested for amino acid sequence and modification specificity (H3K23ac) by ELISA.

FIG. 12 shows the sequences of four peptides identified in hydroxyapatite fractions exhibiting H3 cleavage activity along with the amino acid sequence of mouse Cathepsin L preproprotein (SEQ ID NO:9). To identify the putative H3 protease, proteins in two of the active fractions (#22 and #23) and one of the adjacent non-active fractions (#20) were digested with trypsin. The resulting peptides were analyzed by nano-flow HPLC interfaced with a linear ion trap-Fourier transform mass spectrometer and several thousand collision activated dissociation (CAD) mass spectra were acquired. By searching theses spectra against a database of murine proteins with the SEQUEST algorithm, more than 1,000 peptides from 80-100 proteins were identified in each fraction. Four low level tryptic peptides (0.5%) detected in each of the two active fractions, but not in the inactive fraction, matched the mature form of the lysosomal cysteine protease, Cathepsin L. All were present at or below the 0.5% abundance level. Sequences for the detected peptides are ENGGLDSEESYPYEAK (SEQ ID NO:26), NSWGSEWGMEGYIK (SEQ ID NO:27), DRDNHCGLATAASYPVVN (SEQ ID NO:28), and DNHCGLATAASYPVVN (SEQ ID NO:29). The corresponding residues are underlined within the full amino acid sequence of Cathepsin L. No other proteases were detected in the above analyses.

FIG. 13 is an immunoblot showing inhibition of recombinant mouse Cathepsin L by the cysteine protease inhibitor, E64. Recombinant mouse Cathepsin L was incubated with C-terminally HIS-tagged recombinant histone H3 at both pH 7.5 (left) and pH 5.5 (right) in either buffer alone (−) or in the presence of the cysteine protease inhibitor E64 (+); the final concentration of E64 was 10 μM, 50 μM, and 500 μM.

FIGS. 14A-14C are immunoblots confirming the methylation and acetylation status of rH3-HIS. In FIG. 14A, C-terminally HIS-tagged recombinant histone H3 was mutated to K27C and then alkylated to convert K27C to K27me2. An aliquot of this protein was then treated with acetic anhydride to acetylate all free lysines. These proteins were assayed for the presence of K27me2 by immunoblot. The key applies to FIGS. 14A-14C. The same recombinant histone H3 proteins were also assayed for the presence of H3K18ac (FIG. 14B) and H3K23ac (FIG. 14C).

FIGS. 15A-15C show the effect of in vivo chemical inhibition of Cathepsin L on the transcriptional profile of differentiating ESCs. Cells were differentiated with RA in the presence of either Cathepsin L I inhibitor or DMSO alone, as in FIG. 6B. mRNA was isolated from those cells at the time points indicated, reverse transcribed into cDNA, and assayed for the expression of specific genes by Q-PCR with SYBR green. The data shown in FIGS. 15A-15C represent the average of two independent experiments. As shown in FIG. 15A, expression of the pluripotency marker Nanog decreases upon differentiation, as expected, and does not differ significantly between inhibitor treated and control cells. Some markers for neuronal/ectodermal differentiation showed slight differences in their expression pattern upon Cathepsin L inhibition (FIG. 15B). Both Nestin and Pax3 trend toward maintaining higher expression after 3 days of differentiation with Cathepsin L I inhibitor versus control cells in which expression peaks at 3 days and then decreases. Other markers of neuronal ectodermal differentiation did not show any significant trend (Musashi, HoxA1). Q-PCR for the genes Sox17 and Gata6, markers of endoderm, showed decreased expression with inhibitor treatment compared to control for both genes, although the trend in expression (increasing with differentiation) was the same for treated and control cells. Interestingly, Q-PCR for markers of mesodermal lineage, including Myf5 and Fgf8, did not show significant expression.

### DETAILED DESCRIPTION OF THE INVENTION

The present invention relates generally to methods of modulating histone proteolysis. Accordingly a first aspect of the present invention is directed to a method of administering to a cell an agent that modulates histone proteolysis at an amino acid motif comprising KQLATK (SEQ ID NO:4) of the histone.

In a preferred embodiment of the present invention, the agent modulates histone proteolysis of histone-3 at an amino acid motif comprising KQLATK. The full length amino acid sequence of histone 3 is set forth below in SEQ ID NO:1:

```
Ala Arg Thr Lys Gln Thr Ala Arg Lys Ser Thr Gly Gly Lys Ala Pro
1 5 10 15
Arg Lys Gln Leu Ala Thr Lys Ala Ala Arg Lys Ser Ala Pro Ala Thr
                        20 25 30
Gly Gly Val Lys Lys Pro His Arg Tyr Arg Pro Gly Thr Val Ala Leu
                35 40 45
Arg Glu Ile Arg Arg Tyr Gln Lys Ser Thr Glu Leu Leu Ile Arg Lys
        50 55 60
Leu Pro Phe Gln Arg Leu Val Arg Glu Ile Ala Gln Asp Phe Lys Thr
65 70 75 80
Asp Leu Arg Phe Gln Ser Ser Ala Val Met Ala Leu Gln Glu Ala Ser
                                85 90 95
Glu Ala Tyr Leu Val Gly Leu Phe Glu Asp Thr Asn Leu Cys Ala Ile
                        100 105 110
His Ala Lys Arg Val Thr Ile Met Pro Lys Asp Ile Gln Leu Ala Arg
                115 120 125
Arg Ile Arg Gly Glu Arg Ala
        130 135
```

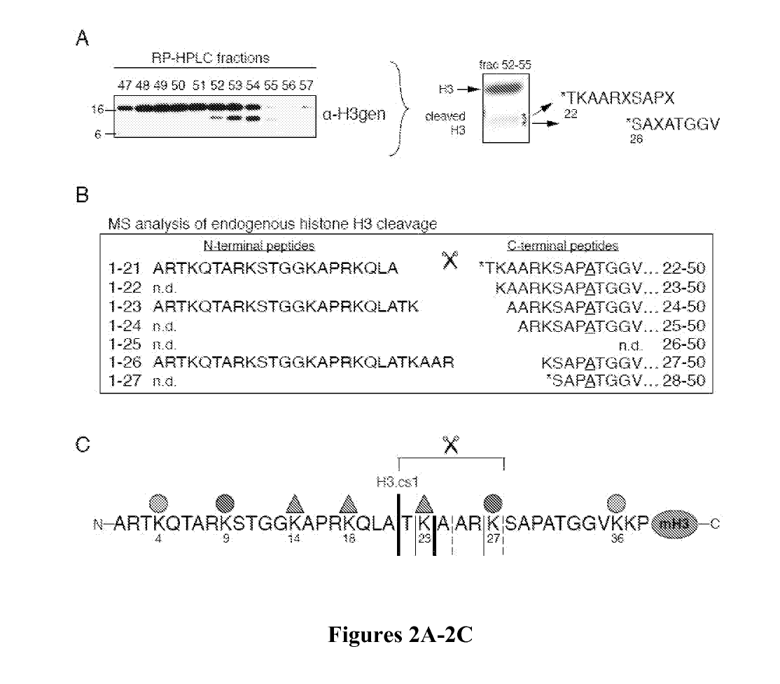

The KQLATK motif (SEQ ID NO:4) of histone 3 is located at amino acids 18-23 of SEQ ID NO:1. Histone proteolysis at a motif comprising KQLATK can occur within or adjacent to the motif. FIG. 2C shows the various cleavage sites within the KQLATK motif of histone 3 (i.e., between amino acids residues 21-22 and 22-23 of SEQ ID NO:1) and adjacent to the KQLATK motif (i.e., between amino acids residues 23-24, 24-25, 26-27 and 27-28 of SEQ ID NO:1), and the resulting N-terminal and C-terminal peptide fragments that are generated are shown in FIG. 2B. In a preferred embodiment of the present invention, the primary cleavage site of histone-3 is located between amino acids 21-22. Cleavage of histone 3 between amino acids residues 21-22 generates the N-terminal histone cleavage product of SEQ ID NO:2 (ARTKQTARKSTGGKAPRKQL) and the C-terminal histone 3 cleavage product of SEQ ID NO:3 (TKAARKSAPATGGVKKPHRYRPGTVALREIRRYQKSTELLIRKLPFQRLVREI AQDFKTDLRFQSSAVMALQEASEAYLVGLFEDTNLCAIHAKRVTIMPKDIQL ARRIRGERA).

In one embodiment of the present invention, the agent that modulates histone proteolysis at a motif comprising KQLATK of the histone is an agent that inhibits cathepsin or cathepsin-mediated proteolysis of a histone. Suitable cathepsin inhibitors include nucleic acid molecules, proteins or peptides, and small molecule inhibitors. In a preferred embodiment, the cathepsin inhibitor is selective for inhibiting cathepsin L mediated histone proteolysis.

Suitable nucleic acid inhibitors of cathepsin for use in the present invention include, but are not limited to, siRNA and antisense molecules. siRNA are double stranded synthetic RNA molecules approximately 20-25 nucleotides in length with short 2-3 nucleotide 3′ overhangs on both ends. The double stranded siRNA molecule represents the sense and anti-sense strand of a portion of the target mRNA molecule, in this case a portion of a cathepsin or, more preferably, a cathepsin L mRNA sequence. siRNA molecules are typically designed to a region of the mRNA target approximately 50-100 nucleotides downstream from the start codon. Upon introduction into a cell, the siRNA complex triggers the endogenous RNA interference (RNAi) pathway resulting in the cleavage and degradation of the target mRNA molecule. Various improvements of siRNA compositions, such as the incorporation of modified nucleosides or motifs into one or both strands of the siRNA molecule to enhance stability, specificity, and efficacy have been described and are suitable for use in accordance with this aspect of the invention (see e.g., WO2004015107 to Giese et al., WO2003070918 to McSwiggen et al., WO199839352 to Imanishi et al., U.S. Patent Application Publication No. 2002/0068708 to Jesper et al.; U.S. Patent Application Publication No. 2002/0147332 to Kaneko et al; and U.S. Patent Application Publication No. 2008/0119427 to Bhat et al., which are all hereby incorporated by reference in their entirety).

siRNA molecules suitable for inhibiting cathepsin L in accordance with the methods of the present invention have a nucleotide sequence of CCGGCCAGCTATCCTGTCGTGAATTCTCGAGAATTCACGACAGGATAGCT GGTTTTTG (SEQ ID NO:7) or CCGGGAATTGCCTCAGCTACTCTAACTCGAGTTAGAGTAGCTGAGGCAAT TCTTTTT (SEQ ID NO:8). Other siRNA molecules directed to the human cathepsin L mRNA sequence are known in the art and are also suitable for use in the methods of the present invention. One such siRNA molecule is directed to nucleotides 91-111 of SEQ ID NO:16 of the human cathepsin L cDNA sequence as described by Zheng et al., “Senescence-Initiated Reversal of Drug Resistance Specific Role of Cathepsin L,” Cancer Research 64:1773-1780 (2004) which is hereby incorporated by reference in its entirety.

Antisense compounds are nucleic acid molecules which specifically hybridize with one or more target nucleic acids encoding cathepsin or cathepsin L protein. “Target nucleic acids” encompass DNA encoding a cathepsin protein, RNA (including pre-mRNA and mRNA) transcribed from such DNA, and also cDNA derived from such RNA. The specific hybridization of an antisense compound with its target nucleic acid interferes with the normal function of the nucleic acid. The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity which may be engaged in or facilitated by the RNA. The overall effect of such interference with target nucleic acid function is modulation of the expression of the cathepsin protein.

Cathepsin L antisense molecules suitable for use in the methods of the present invention include those described by Levicar et al, “Selective Suppression of Cathepsin L by Antisense cDNA Impairs Human Brain Tumor Cell Invasion In Vitro and Promotes Apoptosis,” Cancer Gene Therapy 10:141-151 (2003), which is hereby incorporated by reference in its entirety.

Other cathepsin inhibitors suitable for use in the present invention include synthetic peptides and small molecule inhibitors, many which are known and described in the art. For example, peptide cathepsin L inhibitors suitable for use in the present invention include Z-Phe-Phe-CH₂F and Z-Phe-Tyr-CHO as described by Shaw et al., “The Affinity-Labeling of Cathepsin S with Peptidyl Diazomethyl Ketones. Comparison with the Inhibition of Cathepsin L and Calpain,” FEBS Lett 334:340-2 (1993), which is hereby incorporated by reference, and Z-LLY-CHN₂, Ac-LLnL-CHO-ALLN, aprotinin, and leupeptin. Peptide fluoromethyl ketones, such as N-morpholineurea-phenylalanyl-homophenylalanylfluoromethyl ketone, Z—FF-FMK, Z-LL-FMK, and Z-Phe-Tyr(t-Bu)-diazomethylketone as described by Harth et al., “Peptide Fluoromethyl Ketones Arrest Intracellular Replication and Intercellular Transmission of Trypanosoma cruzi,” Mol Biochem Parasitol 58(10):17-24 (1993) which is hereby incorporated by reference in its entirety, can also be used in the methods of the present invention.

Potent pentapeptide amide inhibitors of cathepsin L, in particular LLLTR-NH₂ (SEQ ID NO:21), RKLLW-NH₂ (SEQ ID NO:22), LFLTR-NH₂ (SEQ ID NO:23), RKLWL-NH₂ (SEQ ID NO:24), and RKLWD-NH₂ (SEQ ID NO:25), as described by Brinker et al., “Highly Potent Inhibitors of Human Cathepsin L Identified by Screening Combinatorial Pentapeptide Amide Collections,” Eur J Biochem 267(16):5085-92 (2000) are also suitable for use in the present invention.

Alpha-ketoamide derivatives, such as N-(quinoline-2-carbonyl)-L-isoleucyl-(3S)-3-amino-2-oxo-4-phenylbutyric acid benzylamide, N—[N-(6-oxo-1,4,5,6-tetrahydropyridazine-3-carbonyl)-L-leucyl]-(3S)-3-amino-2-oxo-4-phenylbutyric acid benzylamide, N-benzyloxycarbonyl-L-leucyl-L-leucyl-(3S)-3-amino-2-oxo-4-phenylbutyric acid benzylamide, or N-(quinoline-2-carbonyl)-L-leucyl-L-leucyl-(3S)-3-amino-2-oxo-4-phenylbutyric acid benzylamide, as described in WO/1996/016079 to Sohda et al., which is hereby incorporated by reference in its entirety, are also suitable for carrying out the methods of the present invention.

Other cathepsin L inhibitors suitable for carrying out the methods of the present invention include acylaminoaldehyde derivatives, such as N-valproyl-(L)-valine (IS)-3-formyl-1-(3-indolylmethyl)-2-propenylamide, N-benzyloxycarbonyl-(L)-alanyl-(L)-alanine (IS)-3-formyl-1-benzyl-2-propenylamide, N-α-naphthalenesulfonyl-(L)-isoleucine (IR)-3-formyl-1-(3-indolylmethyl)propylamide, and N-α-naphthalenesulfonyl-(L)-isoleucine (IR)-3-formyl-1-benzylpropylamide as described in WO/1996/010014 to Sohda et al., which is hereby incorporated by reference in its entirety.

Also suitable for inhibiting cathepsin L in accordance with the methods of the present invention are thiocarbazate derivatives as described by Myers et al., “Identification and Synthesis of Unique Thiocarbazate Cathepsin L Inhibitors,” Bioorganic & Medicinal Chemistry Letters 18(1):210-214 (2008), which is hereby incorporated by reference in its entirety.

In another embodiment of this method of the present invention, the agent that modulates histone proteolysis at an KQLATK motif of the histone is an agent that activates, induces, or enhances cathepsin activity. Exemplary cathepsin “activators” that can be used in the methods of the present invention include glycerol, urea, EDTA, transglutaminase and reducing agents. Urea, and its derivatives, enhance cathepsin L and D activity by a factor of 2.5 and 6, respectively (Wiederanders et al., “The Azocasein-Urea-Pepstatin Assay Discriminates Between Lysosomal Proteinases,” Biomed Biochim Acta 45(11-12):1477-1483 (1986), which is hereby incorporated by reference in its entirety). Ethylenediaminetetraacetic acid (EDTA) enhances cathepsin activity by preventing its inactivation by heavy metals.

Exemplary reducing agents that enhance cathepsin activity include, sulfides, thiols such as dithiothreitol or trithiohexitol, cysteine, N-acetylcysteine, proteins or protein hydrolysates high in cysteine, mercaptoethanol, thioglycerol, thioalkanoic acids, and mercaptocarboxylic acids and analogs thereof such as, for example, mercaptosuccinic acid, thiolactic acid, thioglycolic acid and salts thereof, coenzyme A, or reduced glutathione (GSH). These reducing agents can be administered in their active form or in the form of precursors such as, for example, oxothiazolidine carboxylate.

In another embodiment of the present invention, the agent modulating histone proteolysis at an KQLATK motif of the histone is a recombinant cathepsin protein or proteolytic active cathepsin polypeptide that mimics cathepsin proteolytic activity. Alternatively, the agent is a nucleic acid molecule encoding the recombinant cathepsin protein or proteolytic active cathepsin polypeptide. A proteolytic active cathepsin polypeptide is a polypeptide comprising the protease domain of the enzyme responsible for histone proteolysis. This polypeptide fragment may comprise an intermediate active cathepsin polypeptide or a mature cathepsin polypeptide that are each derived from the full length cathepsin preproprotein. The proteolytic active cathepsin polypeptide may also comprise only the amino acid sequence of the mature cathepsin polypeptide that is responsible for histone proteolysis.

In a preferred embodiment, the recombinant cathepsin protein or proteolytic active cathepsin polypeptide is cathepsin L or derived from cathepsin L preproprotein, or is a nucleic acid molecule encoding the cathepsin L protein or proteolytic active cathepsin polypeptide. The amino acid sequences of cathepsin L preproprotein and cathepsin L intermediate active and mature polypeptides are known in the art and disclosed infra.

Recombinant cathepsin L proteins and methods for making such recombinant proteins have been described in the art (see e.g., the isolated recombinant polypeptides of human cathepsin L described in U.S. Pat. No. 6,737,055 to Bernard et al.; Nomura et al., “Characterization and Crystallization of Recombinant Human Cathepsin L,” Biochem Biophys Res Commun 228(3):792-96 (1996); and Smith et al., “Activity and Deletion Analysis of Recombinant Human Cathepsin L Expressed in Escherichia coli,” J Biol Chem 264(34):20487-495 (1989) which are hereby incorporated by reference in their entirety). Such recombinant proteins and peptides are suitable for use in accordance with this aspect of the present invention. A number of recombinant cathepsin L proteins are also available commercially (see e.g., R&D Systems, Minneapolis, Minn. and Spring Bioscience, Fremont, Calif.).

Recombinant proteolytic active cathepsin L polypeptides can be generated by standard protein biosynthesis or peptide synthesis techniques (i.e., liquid-phase or solid-phase synthesis) using the amino acid sequence of the preproprotein, intermediate, or mature form of the cathepsin L protein.

The amino acid sequence of the preproprotein form of mouse Cathepsin L is set forth below as SEQ ID NO:9.

```
Met Asn Leu Leu Leu Leu Leu Ala Val Leu Cys Leu Gly Thr Ala Leu
1 5 10 15
Ala Thr Pro Lys Phe Asp Gln Thr Phe Ser Ala Glu Trp His Gln Trp
                        20 25 30
Lys Ser Thr His Arg Arg Leu Tyr Gly Thr Asn Glu Glu Glu Trp Arg
                35 40 45
Arg Ala Ile Trp Glu Lys Asn Met Arg Met Ile Gln Leu His Asn Gly
        50 55 60
Glu Tyr Ser Asn Gly Gln His Gly Phe Ser Met Glu Met Asn Ala Phe
65 70 75 80
Gly Asp Met Thr Asn Glu Glu Phe Arg Gln Val Val Asn Gly Tyr Arg
                                85 90 95
His Gln Lys His Lys Lys Gly Arg Leu Phe Gln Glu Pro Leu Met Leu
                        100 105 110
Lys Ile Pro Lys Ser Val Asp Trp Arg Glu Lys Gly Cys Val Thr Pro
                115 120 125
Val Lys Asn Gln Gly Gln Cys Gly Ser Cys Trp Ala Phe Ser Ala Ser
        130 135 140
Gly Cys Leu Glu Gly Gln Met Phe Leu Lys Thr Gly Lys Leu Ile Ser
145 150 155 160
Leu Ser Glu Gln Asn Leu Val Asp Cys Ser His Ala Gln Gly Asn Gln
                                165 170 175
Gly Cys Asn Gly Gly Leu Met Asp Phe Ala Phe Gln Tyr Ile Lys Glu
                        180 185 190
Asn Gly Gly Leu Asp Ser Glu Glu Ser Tyr Pro Tyr Glu Ala Lys Asp
                195 200 205
Gly Ser Cys Lys Tyr Arg Ala Glu Phe Ala Val Ala Asn Asp Thr Gly
        210 215 220
Phe Val Asp Ile Pro Gln Gln Glu Lys Ala Leu Met Lys Ala Val Ala
225 230 235 240
Thr Val Gly Pro Ile Ser Val Ala Met Asp Ala Ser His Pro Ser Leu
                                245 250 255
Gln Phe Tyr Ser Ser Gly Ile Tyr Tyr Glu Pro Asn Cys Ser Ser Lys
                        260 265 270
Asn Leu Asp His Gly Val Leu Leu Val Gly Tyr Gly Tyr Glu Gly Thr
                275 280 285
Asp Ser Asn Lys Asn Lys Tyr Trp Leu Val Lys Asn Ser Trp Gly Ser
        290 295 300
Glu Trp Gly Met Glu Gly Tyr Ile Lys Ile Ala Lys Asp Arg Asp Asn
305 310 315 320
His Cys Gly Leu Ala Thr Ala Ala Ser Tyr Pro Val Val Asn
                                325 330
```

The amino acid sequence of the preproprotein form of human Cathepsin L is set forth below as SEQ ID NO:10.

```
Met Asn Pro Thr Leu Ile Leu Ala Ala Phe Cys Leu Gly Ile Ala Ser
1 5 10 15
Ala Thr Leu Thr Phe Asp His Ser Leu Glu Ala Gln Trp Thr Lys Trp
                        20 25 30
Lys Ala Met His Asn Arg Leu Tyr Gly Met Asn Glu Glu Gly Trp Arg
                35 40 45
Arg Ala Val Trp Glu Lys Asn Met Lys Met Ile Glu Leu His Asn Gln
        50 55 60
Glu Tyr Arg Glu Gly Lys His Ser Phe Thr Met Ala Met Asn Ala Phe
65 70 75 80
Gly Asp Met Thr Ser Glu Glu Phe Arg Gln Val Met Asn Gly Phe Gln
                                85 90 95
Asn Arg Lys Pro Arg Lys Gly Lys Val Phe Gln Glu Pro Leu Phe Tyr
                        100 105 110
Glu Ala Pro Arg Ser Val Asp Trp Arg Glu Lys Gly Tyr Val Thr Pro
                115 120 125
Val Lys Asn Gln Gly Gln Cys Gly Ser Cys Trp Ala Phe Ser Ala Thr
        130 135 140
Gly Ala Leu Glu Gly Gln Met Phe Arg Lys Thr Gly Arg Leu Ile Ser
145 150 155 160
Leu Ser Glu Gln Asn Leu Val Asp Cys Ser Gly Pro Gln Gly Asn Glu
                                165 170 175
Gly Cys Asn Gly Gly Leu Met Asp Tyr Ala Phe Gln Tyr Val Gln Asp
                        180 185 190
Asn Gly Gly Leu Asp Ser Glu Glu Ser Tyr Pro Tyr Glu Ala Thr Glu
                195 200 205
Glu Ser Cys Lys Tyr Asn Pro Lys Tyr Ser Val Ala Asn Asp Thr Gly
        210 215 220
Phe Val Asp Ile Pro Lys Gln Glu Lys Ala Leu Met Lys Ala Val Ala
225 230 235 240
Thr Val Gly Pro Ile Ser Val Ala Ile Asp Ala Gly His Glu Ser Phe
                                245 250 255
Leu Phe Tyr Lys Glu Gly Ile Tyr Phe Glu Pro Asp Cys Ser Ser Glu
                        260 265 270
Asp Met Asp His Gly Val Leu Val Val Gly Tyr Gly Phe Glu Ser Thr
                275 280 285
Glu Ser Asp Asn Asn Lys Tyr Trp Leu Val Lys Asn Ser Trp Gly Glu
        290 295 300
Glu Trp Gly Met Gly Gly Tyr Val Lys Met Ala Lys Asp Arg Arg Asn
305 310 315 320
His Cys Gly Ile Ala Ser Ala Ala Ser Tyr Pro Thr Val
                                325 330
```

The amino acid sequence of the intermediate active form of mouse Cathepsin L is set forth below as SEQ ID NO:11.

```
Glu Pro Leu Met Leu Lys Ile Pro Lys Ser Val Asp Trp Arg Glu Lys
1 5 10 15
Gly Cys Val Thr Pro Val Lys Asn Gln Gly Gln Cys Gly Ser Cys Trp
                        20 25 30
Ala Phe Ser Ala Ser Gly Cys Leu Glu Gly Gln Met Phe Leu Lys Thr
                35 40 45
Gly Lys Leu Ile Ser Leu Ser Glu Gln Asn Leu Val Asp Cys Ser His
        50 55 60
Ala Gln Gly Asn Gln Gly Cys Asn Gly Gly Leu Met Asp Phe Ala Phe
65 70 75 80
Gln Tyr Ile Lys Glu Asn Gly Gly Leu Asp Ser Glu Glu Ser Tyr Pro
                                85 90 95
Tyr Glu Ala Lys Asp Gly Ser Cys Lys Tyr Arg Ala Glu Phe Ala Val
                        100 105 110
Ala Asn Asp Thr Gly Phe Val Asp Ile Pro Gln Gln Glu Lys Ala Leu
                115 120 125
Met Lys Ala Val Ala Thr Val Gly Pro Ile Ser Val Ala Met Asp Ala
        130 135 140
Ser His Pro Ser Leu Gln Phe Tyr Ser Ser Gly Ile Tyr Tyr Glu Pro
145 150 155 160
Asn Cys Ser Ser Lys Asn Leu Asp His Gly Val Leu Leu Val Gly Tyr
                                165 170 175
Gly Tyr Glu Gly Thr Asp Ser Asn Lys Asn Lys Tyr Trp Leu Val Lys
                        180 185 190
Asn Ser Trp Gly Ser Glu Trp Gly Met Glu Gly Tyr Ile Lys Ile Ala
                195 200 205
Lys Asp Arg Asp Asn His Cys Gly Leu Ala Thr Ala Ala Ser Tyr Pro
        210 215 220
Val Val Asn
225
```

The amino acid sequence of the intermediate active form of human Cathepsin L is set forth below as SEQ ID NO:12.

```
Glu Pro Leu Phe Tyr Glu Ala Pro Arg Ser Val Asp Trp Arg Glu Lys
1 5 10 15
Gly Tyr Val Thr Pro Val Lys Asn Gln Gly Gln Cys Gly Ser Cys Trp
                        20 25 30
Ala Phe Ser Ala Thr Gly Ala Leu Glu Gly Gln Met Phe Arg Lys Thr
                35 40 45
Gly Arg Leu Ile Ser Leu Ser Glu Gln Asn Leu Val Asp Cys Ser Gly
        50 55 60
Pro Gln Gly Asn Glu Gly Cys Asn Gly Gly Leu Met Asp Tyr Ala Phe
65 70 75 80
Gln Tyr Val Gln Asp Asn Gly Gly Leu Asp Ser Glu Glu Ser Tyr Pro
                                85 90 95
Tyr Glu Ala Thr Glu Glu Ser Cys Lys Tyr Asn Pro Lys Tyr Ser Val
                        100 105 110
Ala Asn Asp Thr Gly Phe Val Asp Ile Pro Lys Gln Glu Lys Ala Leu
                115 120 125
Met Lys Ala Val Ala Thr Val Gly Pro Ile Ser Val Ala Ile Asp Ala
        130 135 140
Gly His Glu Ser Phe Leu Phe Tyr Lys Glu Gly Ile Tyr Phe Glu Pro
145 150 155 160
Asp Cys Ser Ser Glu Asp Met Asp His Gly Val Leu Val Val Gly Tyr
                                165 170 175
Gly Phe Glu Ser Thr Glu Ser Asp Asn Asn Lys Tyr Trp Leu Val Lys
                        180 185 190
Asn Ser Trp Gly Glu Glu Trp Gly Met Gly Gly Tyr Val Lys Met Ala
                195 200 205
Lys Asp Arg Arg Asn His Cys Gly Ile Ala Ser Ala Ala Ser Tyr Pro
        210 215 220
Thr Val
225
```

The amino acid sequence of the mature active form of mouse Cathepsin L is set forth as SEQ ID NO:13.

```
Ile Pro Lys Ser Val Asp Trp Arg Glu Lys Gly Cys Val Thr Pro Val
1 5 10 15
Lys Asn Gln Gly Gln Cys Gly Ser Cys Trp Ala Phe Ser Ala Ser Gly
                        20 25 30
Cys Leu Glu Gly Gln Met Phe Leu Lys Thr Gly Lys Leu Ile Ser Leu
                35 40 45
Ser Glu Gln Asn Leu Val Asp Cys Ser His Ala Gln Gly Asn Gln Gly
        50 55 60
Cys Asn Gly Gly Leu Met Asp Phe Ala Phe Gln Tyr Ile Lys Glu Asn
65 70 75 80
Gly Gly Leu Asp Ser Glu Glu Ser Tyr Pro Tyr Glu Ala Lys Asp Gly
                                85 90 95
Ser Cys Lys Tyr Arg Ala Glu Phe Ala Val Ala Asn Asp Thr Gly Phe
                        100 105 110
Val Asp Ile Pro Gln Gln Glu Lys Ala Leu Met Lys Ala Val Ala Thr
                115 120 125
Val Gly Pro Ile Ser Val Ala Met Asp Ala Ser His Pro Ser Leu Gln
        130 135 140
Phe Tyr Ser Ser Gly Ile Tyr Tyr Glu Pro Asn Cys Ser Ser Lys Asn
145 150 155 160
Leu Asp His Gly Val Leu Leu Val Gly Tyr Gly Tyr Glu Gly Thr Asp
                                165 170 175
Ser Asn Lys Asn Lys Tyr Trp Leu Val Lys Asn Ser Trp Gly Ser Glu
                        180 185 190
Trp Gly Met Glu Gly Tyr Ile Lys Ile Ala Lys Asp Arg Asp Asn His
                195 200 205
Cys Gly Leu Ala Thr Ala Ala Ser Tyr Pro Val Val Asn
        210 215 220
```

The amino acid sequence of the mature active form of human Cathepsin L is set forth as SEQ ID NO:14.

```
Ala Pro Arg Ser Val Asp Trp Arg Glu Lys Gly Tyr Val Thr Pro Val
1 5 10 15
Lys Asn Gln Gly Gln Cys Gly Ser Cys Trp Ala Phe Ser Ala Thr Gly
                        20 25 30
Ala Leu Glu Gly Gln Met Phe Arg Lys Thr Gly Arg Leu Ile Ser Leu
                35 40 45
Ser Glu Gln Asn Leu Val Asp Cys Ser Gly Pro Gln Gly Asn Glu Gly
        50 55 60
Cys Asn Gly Gly Leu Met Asp Tyr Ala Phe Gln Tyr Val Gln Asp Asn
65 70 75 80
Gly Gly Leu Asp Ser Glu Glu Ser Tyr Pro Tyr Glu Ala Thr Glu Glu
                                85 90 95
Ser Cys Lys Tyr Asn Pro Lys Tyr Ser Val Ala Asn Asp Thr Gly Phe
                        100 105 110
Val Asp Ile Pro Lys Gln Glu Lys Ala Leu Met Lys Ala Val Ala Thr
                115 120 125
Val Gly Pro Ile Ser Val Ala Ile Asp Ala Gly His Glu Ser Phe Leu
        130 135 140
Phe Tyr Lys Glu Gly Ile Tyr Phe Glu Pro Asp Cys Ser Ser Glu Asp
145 150 155 160
Met Asp His Gly Val Leu Val Val Gly Tyr Gly Phe Glu Ser Thr Glu
                                165 170 175
Ser Asp Asn Asn Lys Tyr Trp Leu Val Lys Asn Ser Trp Gly Glu Glu
                        180 185 190
Trp Gly Met Gly Gly Tyr Val Lys Met Ala Lys Asp Arg Arg Asn His
                195 200 205
Cys Gly Ile Ala Ser Ala Ala Ser Tyr Pro Thr Val
        210 215 220
```

Alternatively, recombinant cathepsin L proteins or, more preferably, a polypeptide comprising the proteolytic active site of cathepsin L can be generated for use in the methods of the present invention using standard recombinant cloning technology well known in the art. To achieve recombinant cathepsin L protein or polypeptide production, an isolated nucleic acid molecule encoding the recombinant cathepsin L protein or peptide is inserted into an expression vector and transformed into a host cell to facilitate cathepsin L protein or peptide expression and, subsequently, purification. The nucleic acid molecule encoding the recombinant mouse Cathepsin L proenzyme may comprise the nucleotide sequence set forth below as SEQ ID NO:15.

| | |
|---|---|
| atgaatcttt tactcctttt ggctgtcctc tgcttgggaa cagccttagc tactccaaaa | 60 |
| tttgatcaaa cctttagtgc agagtggcac cagtggaagt ccacacacag aagactgtat | 120 |
| ggcacgaatg aggaagagtg gaggagagcg atatgggaga agaacatgag aatgatccag | 180 |
| ctacacaacg gggaatacag caacgggcag cacggctttt ccatggagat gaacgccttc | 240 |
| ggtgacatga ccaatgagga attcaggcag gtggtgaatg gctaccgcca ccagaagcac | 300 |
| aagaagggga ggctttttca ggaaccgctg atgcttaaga tccccaagtc tgtggactgg | 360 |
| agagaaaagg gttgtgtgac tcctgtgaag aaccagggcc agtgcgggtc ttgttgggcg | 420 |
| tttagcgcat cgggttgcct agaaggacag atgttcctta agaccggcaa actgatctca | 480 |
| ctgagtgaac agaaccttgt ggactgttct cacgctcaag gcaatcaggg ctgtaacgga | 540 |
| ggcctgatgg attttgcttt ccagtacatt aaggaaaatg gaggtctgga ctcggaggag | 600 |
| tcttacccct atgaagcgaa ggacggatct tgtaaataca gagccgagtt cgctgtggct | 660 |
| aatgacacag ggttcgtgga tatccctcag caagagaaag ccctcatgaa ggctgtggcg | 720 |
| actgtggggc ctatttctgt tgctatggac gcaagccatc cgtctctcca gttctatagt | 780 |
| tcaggcatct actatgaacc caactgtagc agcaagaacc tcgaccatgg ggttctgttg | 840 |
| gtgggctatg gctatgaagg aacagattca aataagaata aatattggct tgtcaagaac | 900 |
| agctggggaa gtgaatgggg tatggaaggc tacatcaaaa tagccaaaga ccgggacaac | 960 |
| cactgtggac ttgccaccgc ggccagctat cctgtcgtga attga | 1005 |

The nucleic acid molecule encoding the recombinant human cathepsin L proenzyme may comprise the nucleotide sequence set forth below as SEQ ID NO:16.

| | |
|---|---|
| atgaatccta cactcatcct tgctgccttt tgcctgggaa ttgcctcagc tactctaaca | 60 |
| tttgatcaca gtttagaggc acagtggacc aagtggaagg cgatgcacaa cagattatac | 120 |
| ggcatgaatg aagaaggatg gaggagagca gtgtgggaga agaacatgaa gatgattgaa | 180 |
| ctgcacaatc aggaatacag ggaagggaaa cacagcttca caatggccat gaacgccttt | 240 |
| ggagacatga ccagtgaaga attcaggcag gtgatgaatg gctttcaaaa ccgtaagccc | 300 |
| aggaagggga aagtgttcca ggaacctctg ttttatgagg cccccagatc tgtggattgg | 360 |
| agagagaaag gctacgtgac tcctgtgaag aatcagggtc agtgtggttc ttgttgggct | 420 |
| tttagtgcta ctggtgctct tgaaggacag atgttccgga aaactgggag gcttatctca | 480 |
| ctgagtgagc agaatctggt agactgctct gggcctcaag gcaatgaagg ctgcaatggt | 540 |
| ggcctaatgg attatgcttt ccagtatgtt caggataatg gaggcctgga ctctgaggaa | 600 |
| tcctatccat atgaggcaac agaagaatcc tgtaagtaca atcccaagta ttctgttgct | 660 |
| aatgacaccg gctttgtgga catccctaag caggagaagg ccctgatgaa ggcagttgca | 720 |
| actgtggggc ccatttctgt tgctattgat gcaggtcatg agtccttcct gttctataaa | 780 |
| gaaggcattt attttgagcc agactgtagc agtgaagaca tggatcatgg tgtgctggtg | 840 |
| gttggctacg gatttgaaag cacagaatca gataacaata aatattggct ggtgaagaac | 900 |
| agctggggtg aagaatgggg catgggtggc tacgtaaaga tggccaaaga ccggagaaac | 960 |
| cattgtggaa ttgcctcagc agccagctac cccactgtgt ga | 1002 |

The nucleic acid molecule encoding the recombinant intermediate form of mouse cathepsin L may comprise the nucleotide sequence set forth below as SEQ ID NO:17.

| | |
|---|---|
| gaaccgctga tgcttaagat ccccaagtct gtggactgga gagaaaaggg ttgtgtgact | 60 |
| cctgtgaaga accagggcca gtgcgggtct tgttgggcgt ttagcgcatc gggttgccta | 120 |
| gaaggacaga tgttccttaa gaccggcaaa ctgatctcac tgagtgaaca gaaccttgtg | 180 |
| gactgttctc acgctcaagg caatcagggc tgtaacggag gcctgatgga ttttgctttc | 240 |
| cagtacatta aggaaaatgg aggtctggac tcggaggagt cttaccccta tgaagcgaag | 300 |
| gacggatctt gtaaatacag agccgagttc gctgtggcta atgacacagg gttcgtggat | 360 |
| atccctcagc aagagaaagc cctcatgaag gctgtggcga ctgtggggcc tatttctgtt | 420 |
| gctatggacg caagccatcc gtctctccag ttctatagtt caggcatcta ctatgaaccc | 480 |
| aactgtagca gcaagaacct cgaccatggg gttctgttgg tgggctatgg ctatgaagga | 540 |
| acagattcaa ataagaataa atattggctt gtcaagaaca gctggggaag tgaatggggt | 600 |
| atggaaggct acatcaaaat agccaaagac cgggacaacc actgtggact tgccaccgcg | 660 |
| gccagctatc ctgtcgtgaa ttga | 684 |

The nucleic acid molecule encoding the recombinant intermediate form of human Cathepsin L may comprise the nucleotide sequence set forth below as SEQ ID NO:18.

| | |
|---|---|
| gaacctctgt tttatgaggc ccccagatct gtggattgga gagagaaagg ctacgtgact | 60 |
| cctgtgaaga atcagggtca gtgtggttct tgttgggctt ttagtgctac tggtgctctt | 120 |
| gaaggacaga tgttccggaa aactgggagg cttatctcac tgagtgagca gaatctggta | 180 |
| gactgctctg ggcctcaagg caatgaaggc tgcaatggtg gcctaatgga ttatgctttc | 240 |
| cagtatgttc aggataatgg aggcctggac tctgaggaat cctatccata tgaggcaaca | 300 |
| gaagaatcct gtaagtacaa tcccaagtat tctgttgcta atgacaccgg ctttgtggac | 360 |
| atccctaagc aggagaaggc cctgatgaag gcagttgcaa ctgtggggcc catttctgtt | 420 |
| gctattgatg caggtcatga gtccttcctg ttctataaag aaggcattta ttttgagcca | 480 |
| gactgtagca gtgaagacat ggatcatggt gtgctggtgg ttggctacgg atttgaaagc | 540 |
| acagaatcag ataacaataa atattggctg gtgaagaaca gctggggtga agaatggggc | 600 |
| atgggtggct acgtaaagat ggccaaagac cggagaaacc attgtggaat tgcctcagca | 660 |
| gccagctacc ccactgtgtg a | 681 |

The nucleic acid molecule encoding the recombinant mature form of mouse Cathepsin L may comprise the nucleotide sequence set forth below as SEQ ID NO:19.

| | |
|---|---|
| atccccaagt ctgtggactg gagagaaaag ggttgtgtga ctcctgtgaa gaaccagggc | 60 |
| cagtgcgggt cttgttgggc gtttagcgca tcgggttgcc tagaaggaca gatgttcctt | 120 |
| aagaccggca aactgatctc actgagtgaa cagaaccttg tggactgttc tcacgctcaa | 180 |
| ggcaatcagg gctgtaacgg aggcctgatg gattttgctt tccagtacat taaggaaaat | 240 |
| ggaggtctgg actcggagga gtcttacccc tatgaagcga aggacggatc ttgtaaatac | 300 |
| agagccgagt tcgctgtggc taatgacaca gggttcgtgg atatccctca gcaagagaaa | 360 |
| gccctcatga aggctgtggc gactgtgggg cctatttctg ttgctatgga cgcaagccat | 420 |
| ccgtctctcc agttctatag ttcaggcatc tactatgaac ccaactgtag cagcaagaac | 480 |
| ctcgaccatg gggttctgtt ggtgggctat ggctatgaag gaacagattc aaataagaat | 540 |
| aaatattggc ttgtcaagaa cagctgggga agtgaatggg gtatggaagg ctacatcaaa | 600 |
| atagccaaag accgggacaa ccactgtgga cttgccaccg cggccagcta tcctgtcgtg | 660 |
| aattga | 666 |

The nucleic acid molecule encoding the recombinant mature form of human Cathepsin L protein may comprise the nucleotide sequence set forth below as SEQ ID NO:20.

| | |
|---|---|
| gcccccagat ctgtggattg gagagagaaa ggctacgtga ctcctgtgaa gaatcagggt | 60 |
| cagtgtggtt cttgttgggc ttttagtgct actggtgctc ttgaaggaca gatgttccgg | 120 |
| aaaactggga ggcttatctc actgagtgag cagaatctgg tagactgctc tgggcctcaa | 180 |
| ggcaatgaag gctgcaatgg tggcctaatg gattatgctt tccagtatgt tcaggataat | 240 |
| ggaggcctgg actctgagga atcctatcca tatgaggcaa cagaagaatc ctgtaagtac | 300 |
| aatcccaagt attctgttgc taatgacacc ggctttgtgg acatccctaa gcaggagaag | 360 |
| gccctgatga aggcagttgc aactgtgggg cccatttctg ttgctattga tgcaggtcat | 420 |
| gagtccttcc tgttctataa agaaggcatt tattttgagc cagactgtag cagtgaagac | 480 |
| atggatcatg gtgtgctggt ggttggctac ggatttgaaa gcacagaatc agataacaat | 540 |
| aaatattggc tggtgaagaa cagctggggt gaagaatggg gcatgggtgg ctacgtaaag | 600 |
| atggccaaag accggagaaa ccattgtgga attgcctcag cagccagcta ccccactgtg | 660 |
| tga | 663 |

An isolated nucleic acid molecule encoding the cathepsin L protein or proteolytic active cathepsin polypeptide is inserted into an expression system to which the molecule is heterologous. The heterologous nucleic acid molecule is inserted into the expression system or vector in proper sense (5′→3′) orientation relative to the promoter and any other 5′ regulatory molecules, and correct reading frame. Preparation of the Nucleic Acid Constructs can be Carried Out Using Standard Cloning methods well known in the art as described by SAMBROOK AND RUSSELL, MOLECULAR CLONING: A LABORATORY MANUAL (Cold Springs Laboratory Press, 1989), which is hereby incorporated by reference in its entirety. U.S. Pat. No. 4,237,224 to Cohen and Boyer, which is hereby incorporated by reference in its entirety, also describes the production of expression systems in the form of recombinant plasmids using restriction enzyme cleavage and ligation with DNA ligase.

Suitable expression vectors include those which contain replicon and control sequences that are derived from species compatible with the host cell. For example, if E. coli is used as a host cell, plasmids such as pUC19, pUC18, or pBR322 may be used. Recombinant cathepsin L protein, or an active peptide thereof, can also be expressed and purified using a baculovirus system. Appropriate transfer vectors compatible with insect host cells include, pVL1392, pVL1393, pAcGP67, pAcSecG2T, pAcGHLT, and pAcHLT (BD Biosciences, Franklin Lakes, N.J.). Appropriate viral vectors include adenovirus, adeno-associated virus, retrovirus, lentivirus, and herpes virus vectors. Other suitable expression vectors are described in SAMBROOK AND RUSSELL, MOLECULAR CLONING: A LABORATORY MANUAL (Cold Springs Laboratory Press, 1989), which is hereby incorporated by reference in its entirety. Many known techniques and protocols for manipulation of nucleic acids, for example in preparation of nucleic acid constructs, mutagenesis, sequencing, introduction of DNA into cells and gene expression, and analysis of proteins, are described in detail in CURRENT PROTOCOLS IN MOLECULAR BIOLOGY (Fred M. Ausubel et al. eds., 1992), which is hereby incorporated by reference in its entirety.

Different genetic signals and processing events control many levels of gene expression (e.g., DNA transcription and messenger RNA (“mRNA”) translation). Transcription of DNA is dependent upon the presence of a promoter, which is a DNA sequence that directs the binding of RNA polymerase, and thereby promotes mRNA synthesis. Promoters vary in their “strength” (i.e., their ability to promote transcription). For the purposes of expressing a cloned gene, in this case cathepsin L, it is desirable to use strong promoters to obtain a high level of transcription and, hence, expression and kinase activity. Therefore, depending upon the host system utilized, any one of a number of suitable promoters may also be incorporated into the expression vector carrying the nucleic acid molecules of the present invention. For instance, when using E. coli, its bacteriophages, or plasmids, promoters such as the T7 phage promoter, lac promoter, trp promoter, recA promoter, ribosomal RNA promoter, the P_{R} and P_{L} promoters of coliphage lambda and others, including but not limited, to lacUV5, ompF, bla, lpp, and the like, may be used to direct high levels of transcription of adjacent DNA segments. Additionally, a hybrid trp-lacUV5 (tac) promoter or other E. coli promoters produced by recombinant DNA or other synthetic DNA techniques may be used to provide for transcription of the inserted gene. When using insect cells, suitable baculovirus promoters include late promoters, such as 39K protein promoter or basic protein promoter, and very late promoters, such as the p10 and polyhedron promoters. In some cases it may be desirable to use transfer vectors containing multiple baculovirus promoters.

Translation of mRNA in prokaryotes depends upon the presence of the proper prokaryotic signals, which differ from those of eukaryotes. Efficient translation of mRNA in prokaryotes requires a ribosome binding site called the Shine-Dalgarno (“SD”) sequence on the mRNA. This sequence is a short nucleotide sequence of mRNA that is located before the start codon, usually AUG, which encodes the amino-terminal methionine of the protein. The SD sequences are complementary to the 3′-end of the 16S rRNA (ribosomal RNA) and probably promote binding of mRNA to ribosomes by duplexing with the rRNA to allow correct positioning of the ribosome. For a review on maximizing gene expression, see Roberts and Lauer, Methods in Enzymology, 68:473 (1979), which is hereby incorporated by reference in its entirety.

Host cells suitable for expressing or propagating the nucleic acid construct encoding cathepsin L include any one of the more commonly available gram negative bacteria. Suitable microorganisms include Pseudomonas aeruginosa, Escherichia coli, Salmonella gastroenteritis (typhimirium), S. typhi, S. enteriditis, Shigella flexneri, S. sonnie, S dysenteriae, Neisseria gonorrhoeae, N. meningitides, Haemophilus influenzae, H. pleuropneumoniae, Pasteurella haemolytica, P. multilocida, Legionella pneumophila, Treponema pallidum, T. denticola, T. orales, Borrelia burgdorferi, Borrelia spp., Leptospira interrogans, Klebsiella pneumoniae, Proteus vulgaris, P. morganii, P. mirabilis, Rickettsia prowazeki, R. typhi, R. richettsii, Porphyromonas (Bacteriodes) gingivalis, Chlamydia psittaci, C. pneumoniae, C. trachomatis, Campylobacter jejuni, C. intermedis, C. fetus, Helicobacter pylori, Francisella tularenisis, Vibrio cholerae, Vibrio parahaemolyticus, Bordetella pertussis, Burkholderie pseudomallei, Brucella abortus, B. susi, B. melitens is, B. can is, Spirillum minus, Pseudomonas mallei, Aeromonas hydrophile, A salmonicida, and Yersinia pestis.

In addition to bacteria cells, eukaryotic cells such as mammalian, insect, and yeast systems are also suitable host cells for transfection/transformation of the expression vector carrying an isolated nucleic acid molecule encoding cathepsin L protein or active peptide. Mammalian cell lines available in the art for expression of a heterologous polypeptide include Chinese hamster ovary cells, HeLa cells, baby hamster kidney cells, COS cells and many others. Suitable insect cell lines include those susceptible to baculoviral infection, including Sf9 and Sf21 cells.

Methods for transforming/transfecting host cells with expression vectors are well-known in the art and depend on the host system selected, as described in SAMBROOK AND RUSSELL, MOLECULAR CLONING: A LABORATORY MANUAL (Cold Springs Laboratory Press, 1989), which is hereby incorporated by reference in its entirety. For bacterial cells, suitable techniques may include calcium chloride transformation, electroporation, and transfection using bacteriophage For eukaryotic cells, suitable techniques may include calcium phosphate transfection, DEAE-Dextran, electroporation, liposome-mediated transfection and transduction using retrovirus or other virus, e.g., vaccinia or, for insect cells, baculovirus. For insect cells, the transfer vector containing the nucleic acid construct encoding the cathepsin L protein or active peptide is co-transfected with baculovirus DNA, such a AcNPV, to facilitate homologous recombination between the cathepsin L construct in the transfer vector and baculovirus DNA and the production of a recombinant virus. Subsequent recombinant viral infection of Sf cells results in a high rate of recombinant protein production that can be readily purified using standard purification methods known in the art.

In yet another embodiment of this method of the present invention, the agent that modulates histone proteolysis at an KQLATK motif of the histone is an agent that modulates amino acid acetylation. Endogenously regulated amino acid acetylation occurs via histone acetyltransferases (“HATs”) and histone deacetylases (“HDACs”). HATs acetylate conserved lysine amino acids on histone proteins by transferring an acetyl group from acetyl CoA to a lysine, and HDACs catalyze the removal of an acetyl group from an amino acid. In a preferred embodiment of the present invention, acetylation of one or more lysines in the KQLATK motif modulate histone proteolysis. In a more preferred embodiment of the present invention, acetylation of one or both lysines in the KQLATK motif located at amino acid positions 18 and 23 of SEQ ID NO:1, modulates histone proteolysis.

Specific HAT and HDAC inhibitors for modulating endogenous amino acid acetylation are well known in the art and suitable for carrying out this method of the present invention.

HAT inhibitors useful for modulating histone proteolysis include any of those known in the art, including, but not limited to, coenzyme A conjugates as disclosed in U.S. Pat. No. 6,369,030 to Cole et al., which is hereby incorporated by reference in its entirety; polyisoprenylated benzophenones, such as garcinol, as disclosed in U.S. Published Patent Application No. 2007/0254961 to Tapas Kumar et al., which is hereby incorporated by reference in its entirety; quinoline derivatives, such as MC1626, as described by Smith et al., “Quinoline Derivative MC1626, a Putative GC5N Histone Acetyltransferase (HAT) Inhibitor, Exhibits HAT-Independent Activity Against Toxoplasma gondii,” Antimicrobial Agents and Chemotherapy 51:1109-11 (2007), which is hereby incorporated by reference in its entirety; curcumin and its derivatives; and isothiazolones as described by Stimson et al., “Isothiazolones as Inhibitors of PCAF and p300 Histone Acetyltransferase Activity,” Mol Cancer Ther 4:1521-32 (2005), which is hereby incorporated by reference in its entirety.

Histone deacetylase inhibitors suitable for use in the present invention include nucleoplasmin, chamydocin, Cyl-2, cyclic(eta-oxo-alpha-aminooxiraneoctanoylphenylalanylleucyl-2-piperidinecarbonyl (WF-3161), depudecin, radicocol, oxamfiatin, apidicin, suberoxylanilide hydroxamic acid, and 2-amino-8-oxo-9,10-epoxy-decanoic acid as disclosed in U.S. Patent Application Publication No. 2005/0222013 to Mira et al., which is hereby incorporated by reference in its entirety. In addition, U.S. Pat. No. 6,376,508 to Li, which is hereby incorporated by reference in its entirety, discloses the use of butyrate, trapoxin analogs and trichostatin A as potent HDAC inhibitors that are also suitable for use in the present invention. Other HDAC inhibitors known in the art that are suitable for use in the present invention include, valproic acid and its derivatives as disclosed in U.S. Pat. No. 7,265,154 to Gottlicher; carbamic acid compounds comprising sulfonamide linkages as disclosed in U.S. Pat. No. 6,888,027 to Watkins; compounds having a zinc-binding moiety, such as, a hydroxamic acid group, as disclosed in U.S. Pat. No. 6,495,716 to Lan-Hargest et al.; cyclic tetrapeptide derivatives disclosed in U.S. Pat. No. 6,825,317 to Nishino; and any of the HDAC inhibitory compounds disclosed in U.S. Pat. Nos. 7,399,884, 7,381,825, 7,375,228, 7,169,801, 7,154,002 all to Bressi, which are all hereby incorporated by reference in their entirety. Other HDAC inhibitors, including m-carboxycinnamic acid bis-hydroxamie and the bicyclic depsipeptide, FK228, described by Adachi et al., “Synergistic Effect of Histone Deacetylase Inhibitors FK228 and m-Carboxycinnamic Acid Bis-Hydroxamide with Proteasome Inhibitors PSI and PS-341 Against Gastrointestinal Adenocarcinoma Cells,” Clinical Cancer Research 10:3853-62 (2004); the benzamide, M344, as described by Riessland et al., “The Benzamide M344, A Novel Histone Deacetylase Inhibitor, Significantly Increases SMN2 RNA/Protein Levels in Spinal Muscular Atrophy Cells,” Hum Genet. 120(10):101-110 (2006); and 3-(4-aroyl-2-pyrrolyl)-N-hydroxy-2-propenamides as described by Massa et al., “3-(4-aroyl-1H-pyrrol-2-yl)-N-hydroxy-2-propenamides, A New Class of Synthetic Histone Deacetylase Inhibitors,” J Med Chem 44(13):2069-72 (2001), which are all hereby incorporated by reference in their entirety, are also suitable for use in the present invention.

Another aspect of the present invention is directed to a method of administering to a cell an agent that inhibits histone proteolysis in the cell. The agent administered to the cell is a cathepsin inhibitor selected from the group consisting of a nucleic acid, a peptide, or a small molecule cathepsin inhibitor. Any of the cathepsin inhibitors described supra are suitable for use in accordance with this aspect of the invention. In a preferred embodiment, the cathepsin inhibitor is a cathepsin L inhibitor and histone 3 proteolysis is inhibited.

A related aspect of the present invention is directed to a method of administering to a cell an agent that induces histone proteolysis in the cell. In accordance with this aspect of the present invention, the agent administered to the cell is a recombinant cathepsin protein or proteolytic active cathepsin polypeptide, or a nucleic acid molecule encoding the recombinant cathepsin protein or proteolytic active cathepsin polypeptide as described supra. An agent that induces or activates cathepsin activity may also be administered to the cell to induce histone proteolysis in the cell. In a preferred embodiment, the recombinant cathepsin protein or polypeptide fragment is a cathepsin L recombinant protein or polypeptide, and histone 3 proteolysis is induced in the cell.

A second aspect of the present invention is directed to a method of regulating stem cell differentiation. This method involves administering to a population of stem cells an agent that modulates histone proteolysis under conditions effective to regulate stem cell differentiation. In a preferred embodiment, the agent modulates histone proteolysis at an KQLATK motif (SEQ ID NO:4) of the histone. More preferably, the agent modulates histone proteolysis of histone-3.

In accordance with this aspect of the present invention, an agent that inhibits histone proteolysis is administered to suppress stem cell differentiation. Suitable agents that inhibit histone proteolysis and suppress stem cell differentiation include any of the cathepsin inhibitors, in particular, the cathepsin L inhibitors described supra. Alternatively, the agent may be a histone deacetylase inhibitor. Any of the above mentioned histone deacetylase inhibitors are suitable for suppressing stem cell differentiation.

In another embodiment, an agent that induces histone proteolysis is administered to promote stem cell differentiation. Suitable agents that induce histone proteolysis and promote stem cell differentiation include those agents that induce, enhance, or mimic the activity of cathepsin, in particular agents that induce, enhance, or mimic cathepsin L proteolytic activity. Any of the cathepsin activators, recombinant cathepsin proteins or polypeptides, or nucleic acid molecules encoding a recombinant cathepsin protein or polypeptide as described supra are suitable to induce histone proteolysis. A recombinant cathepsin L protein or proteolytic active polypeptide, or the nucleic acid molecule encoding cathepsin L or the proteolytic active polypeptide are also suitable for use. Other agents suitable for inducing histone proteolysis in accordance with this aspect of the present invention include histone acetyltransferase inhibitors. Any of the histone acetyltransferase inhibitors described supra can be administered to promote stem cell differentiation.

The agents of the present invention can be administered to a population of stem cells in vitro or in vivo.

The agents of the present invention can be administered to any population of adult or embryonic stem cells known in the art. For example, the population of stem cells may comprise a population of primitive hematopoietic stem cells, where the administration of the agents of the present invention enhance the repopulation of hematopoietic and mature blood cell lineages. Enhancing the repopulation of hematopoietic and/or mature blood cell lineages may be desirable to replenish a loss of such cells resulting from disease (e.g., erythrocytopenia, erthrodegenerative disorder, erythroblastopenia, leukoerythroblastosis, erythroclasis, thalassemia and anemia) or following chemotherapy or radiotherapy treatments. Repopulation of hematopoietic stem cell lineages is also desirable in a subject having an autoimmune or immunodeficiency mediated disease. Alternatively, the population of stem cells may comprise a population of mesenchymal stem cells. Agents of the present invention can be administered to promote or suppress mesenchymal stem cell differentiation into specific types of mesenchymal or connective tissues including adipose, osseous, cartilaginous, elastic, muscular, and fibrous connective tissue. Enhancing the differentiation of mesenchymal stem cells is desirable to promote and direct tissue regeneration, for example, cartilage or skin regeneration, muscle morphogenesis, and bone and stromal cell reconstitution. In another embodiment, the population of stem cells comprises a population of neuronal progenitor stem cells. The agents of the present invention can be administered to a population of neural progenitor cells to promote or suppress their differentiation into specific types of neuronal cells including, any type of neuron, glial cell, oligodendrocyte, or astrocyte. Enhancing the differentiation of neuronal progenitor cells is desirable to promote regeneration of a specific functional neuronal cell population which has been lost as a result of disease (e.g., Huntington's Disease, Parkinson's Disease, other neurodegenerative diseases) or injury.

A third aspect of the present invention relates to a method of modulating gene transcription in a cell. This method involves administering to a population of cells an agent that modulates histone proteolysis under conditions effective to modulate gene transcription in the cell. In a preferred embodiment, the agent modulates histone proteolysis at an KQLATK motif (SEQ ID NO:4) of the histone. More preferably, the agent modulates histone proteolysis of histone-3.

Histone proteolysis at or adjacent to a KQLATK motif generates a new N-terminus to the histone protein. This new N-terminus can alter recruitment of effector molecules (i.e., remove the steric hindrance of the previous N-terminal tail allowing effector molecule access) which subsequently alters the gene transcription controlled by such effector molecules. As demonstrated herein, histone-3 cleavage greatly diminishes the ability of the chromodomain-containing protein, Polycomb, from binding to the methylated K27 of histone 3. Polycomb binding causes chromatin remodeling resulting in modified gene transcription. Accordingly, modulation of histone proteolysis may alter the expression of genes associated with Polycomb induced chromatin remodeling including, but not limited to, Nanog, Nestin, Pax3, Musashi, HoxA, GATA6 and Sox17.

In one embodiment, agents that inhibit histone proteolysis are administered to modulate gene transcription in the cell. Any of the cathepsin L and histone deacetylase inhibitors discussed supra are suitable for modulating gene transcription in accordance with this aspect of the present invention.

In an alternative embodiment, agents that induce histone proteolysis, including agents that induce, enhance, or mimic cathepsin L activity, recombinant cathepsin L proteins or proteolytic active polypeptides, nucleic acid molecules encoding recombinant cathepsin L proteins or proteolytic active polypeptides, or histone acetyltransferase inhibitors as discussed supra are suitable for administration to modulate gene transcription in accordance with this aspect of the present invention.

Modulation of gene transcription by administering to a cell an agent according to the methods of the present invention can be monitored using any method known in the art for analyzing gene expression. Examples of known methods for analyzing gene expression include DNA arrays or microarrays (Brazma and Vilo, FEBS Letters 480:17-24 (2000); Celis et al., FEBS Letters 480:2-16 (2000), which are hereby incorporated by reference in their entirety); SAGE (serial analysis of gene expression) (Madden et al., Drug Discov. Today 5:415-425 (2000)), READS (restriction enzyme amplification of digested cDNAs) (Prashar and Weissman, Methods Enzymol. 303:258-72 (1999), which is hereby incorporated by reference in its entirety); TOGA (total gene expression analysis) (Sutcliffe et al., Proc. Natl. Acad. Sci. U.S.A. 97:1976-81 (2000), which is hereby incorporated by reference in its entirety); protein arrays and proteomics (Celis et al., FEBS Letters 480:2-16 (2000); Jungblut et al., Electrophoresis 20:2100-10 (1999), which are hereby incorporated by reference in their entirety); expressed sequence tag (EST) sequencing (Celis et al., FEBS Letters 480:2-16 (2000); Larsson et al., J. Biotechnol. 80:143-57 (2000), which are hereby incorporated by reference in their entirety); subtractive RNA fingerprinting (SuRF) (Fuchs et al., Anal. Biochem. 286:91-98 (2000); Larson et al., Cytometry 4:203-208 (2000), which are hereby incorporated by reference in their entirety); subtractive cloning; differential display (DD) (Jurecic and Belmont, Curr. Opin. Microbiol. 3:316-21 (2000), which is hereby incorporated by reference in its entirety); comparative genomic hybridization (Carulli et al., J. Cell Biochem. 31:286-96 (1998), which is hereby incorporated by reference in its entirety); FISH (fluorescent in situ hybridization) techniques (Going and Gusterson, Eur. J. Cancer 35:1895-904 (1999), which is hereby incorporated by reference in its entirety); and mass spectrometry methods (To, K., Comb. Chem. High Throughput Screen 3:235-41 (2000), which is hereby incorporated by reference in its entirety).

The present invention is also directed to antibodies or antigen-binding fragments thereof that selectively bind to a histone-3 cleavage product. In a preferred embodiment, the antibodies of the present invention selectively bind to the C-terminus of the histone-3 cleavage product of SEQ ID NO:2 or the N-terminus of the histone-3 cleavage product of SEQ ID NO:3. The generation and characterization of a preferred antibody of the invention that selectively binds a histone-3 cleavage product is described infra in the Examples.

The antibodies of the present invention can be monoclonal or polyclonal.

Procedures for raising polyclonal antibodies are well known in the art. Typically, such antibodies can be raised by administering a peptide containing the epitope of interest subcutaneously to New Zealand white rabbits which have first been bled to obtain pre-immune serum. Administered antigenic material will contain synthetic surfactant adjuvant pluronic polyols, or pulverized acrylamide gel containing the protein or polypeptide after SDS-polyacrylamide gel electrophoresis. The rabbits are bled approximately every two weeks after the first injection and periodically boosted with the same antigen three times every six weeks. A sample of serum is then collected 10 days after each boost and polyclonal antibodies are recovered by affinity chromatography using the corresponding antigen to capture the antibody. This and other procedures for raising polyclonal antibodies are disclosed in ANTIBODIES: A LABORATORY MANUAL (Harlow et al. eds., 1988), which is hereby incorporated by reference in its entirety.

A monoclonal antibody is obtained from a substantially homogeneous population of antibodies, i.e., the individual antibodies comprising the population are identical except for possible naturally occurring mutations that may be present in minor amounts. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired activity (See U.S. Pat. No. 4,816,567 to Cabilly et al., and Morrison et al., “Chimeric Human Antibody Molecules Mouse Antigen-Binding Domains with Human Constant Region Domains,” Proc. Natl. Acad. Sci. USA, 81:6851-6855 (1984) which are hereby incorporated by reference in their entirety).

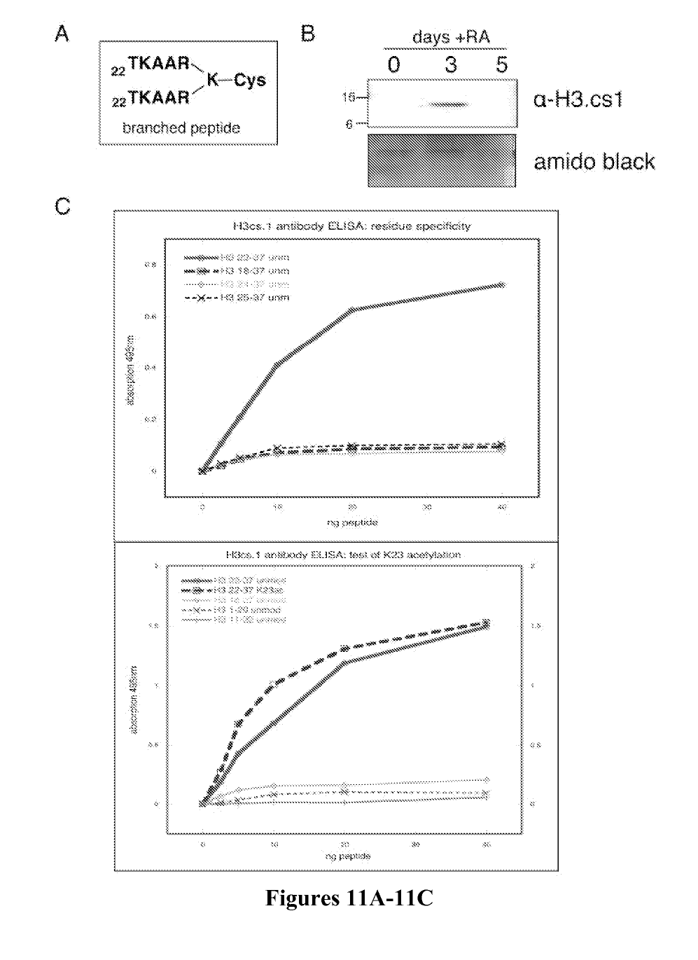

Monoclonal antibodies may be prepared using hybridoma methods, such as those described by Kohler et al., “Continuous Cultures of Fused Cells Secreting Antibody of Predefined Specificity,” Nature 256:495-7 (1975) or ANTIBODIES: A LABORATORY MANUAL (Harlow et al. eds., 1988), which are hereby incorporated by reference in their entirety. In a hybridoma method, a mouse or other appropriate host animal, is typically immunized with an immunizing agent to elicit lymphocytes that produce or are capable of producing antibodies that will specifically bind to the immunizing agent. Alternatively, the lymphocytes may be immunized in vitro. In accordance with the present invention, the immunizing agent comprises a histone cleavage product; preferably, a histone-3 cleavage product; more preferably, the histone-3 cleavage product of SEQ ID NO:2 or SEQ ID NO:3. Alternatively, the immunizing agent comprises the branched H3 cleavage peptide shown in FIG. 11A.

In addition to the traditional approaches for generating monoclonal antibodies, which depend on the availability of purified protein or peptide for use as the immunogen, more recently developed DNA based immunizations are also suitable for generating the antibodies of the present invention. In this approach, DNA-based immunization can be used, wherein DNA encoding a histone cleavage product is expressed as a fusion protein with human IgG1 and injected into the host animal according to methods known in the art (e.g., Kilpatrick et al., “Gene Gun Delivered DNA-Based Immunizations Mediate Rapid Production of Murine Monoclonal Antibodies to the Flt-3 Receptor,” Hybridoma 17(6):569-76 (1998) and Kilpatrick et al., “High-Affinity Monoclonal Antibodies to PED/PEA-15 Generated Using 5 Micrograms of DNA,” Hybridoma 19(4):297-302 (2000), which are hereby incorporated by reference in their entirety). Alternatively, the nucleic acid sequence encoding the histone cleavage product be can expressed in a baculovirus expression system. The advantages to this system include ease of generation, high levels of expression, and post-translational modifications that are highly similar to those seen in mammalian systems.

Generally, peripheral blood lymphocytes are used in methods of producing monoclonal antibodies if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (JAMES W. GODING, MONOCLONAL ANTIBODIES: PRINCIPLES AND PRACTICE (Academic Press 1986), which is hereby incorporated by reference in its entirety). Immortalized cell lines are usually transformed mammalian cells, including myeloma cells of rodent, bovine, equine, and human origin. The hybridoma cells are cultured in a suitable culture medium that preferably contains one or more substances that inhibit the growth or survival of the unfused, immortalized cells. Preferred immortalized cell lines (e.g., murine myeloma lines) are those that fuse efficiently and support stable high level expression of antibody by the selected antibody-producing cells. Human myeloma and mouse-human heteromyeloma cell lines have also been described for the production of human monoclonal antibodies (Kozbor et al., “A Human Hybrid Myeloma for Production of Human Monoclonal Antibodies,” J. Immunol. 133:3001-5 (1984) and MONOCLONAL ANTIBODY PRODUCTION TECHNIQUES AND APPLICATIONS (L. B. Shook ed., 1987), which are hereby incorporated by reference in their entirety). The culture medium in which the hybridoma cells are cultured can be assayed for the presence of monoclonal antibodies directed against the histone cleavage product. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA), or chemiluminescence assays. Such techniques and assays are known in the art, and are fully described in ANTIBODIES: A LABORATORY MANUAL (Harlow et al. eds., 1988), which is hereby incorporated by reference in its entirety.

After the desired hybridoma cells are identified, the clones may be subcloned by limiting dilution or FACS sorting procedures and grown by standard methods. Suitable culture media for this purpose include, for example, Dulbecco's Modified Eagle's Medium and RPMI-1640 medium. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-Sepharose, protein G, hydroxylapatite chromatography, gel electrophoresis, dialysis, or affinity chromatography.

The monoclonal antibodies of the present invention may also be made by recombinant DNA methods, such as those described in U.S. Pat. No. 4,816,567 to Cabilly et al, which is hereby incorporated by reference in its entirety. DNA encoding the monoclonal antibodies can be readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as simian COS cells, Chinese hamster ovary (CHO) cells, plasmacytoma cells, or myeloma cells that do not otherwise produce immunoglobulin protein, to obtain the synthesis of monoclonal antibodies in the recombinant host cells, using the appropriate vectors described herein. The DNA also may be modified, for example, by substituting the coding sequence for human heavy and light chain constant domains in place of the homologous murine sequences or by covalently joining to the immunoglobulin coding sequence all or part of the coding sequence for a non-immunoglobulin polypeptide (See U.S. Pat. No. 4,816,567 to Cabilly et al, which is hereby incorporated by reference in its entirety). Optionally, such a non-immunoglobulin polypeptide is substituted for the constant domains of an antibody or substituted for the variable domains of one antigen-combining site of an antibody to create a chimeric bivalent antibody comprising one antigen-combining site having specificity for the histone cleavage product and another antigen-combining site having specificity for a different antigen.

The antibodies of the present invention may be whole immunoglobulin (i.e., an intact antibody) of any class. Native antibodies are usually heterotetrameric glycoproteins, composed of two identical light (L) chains and two identical heavy (H) chains. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V(H)) followed by a number of constant domains. Each light chain typically has a variable domain at one end (V(L)) and a constant domain at its other end. Depending on the amino acid sequence of the constant domain of their heavy chains, immunoglobulins can be assigned to different classes. There are five major classes of human immunoglobulins: IgA, IgD, IgE, IgG, and IgM, and several of these may be further divided into subclasses (isotypes), e.g., IgG-1, IgG-2, IgG-3, and IgG-4; IgA-1 and IgA-2. The antigen-binding domain of the antibody is cleft formed by the variable regions of the heavy and light chains. Antibodies of the present invention can be mono-, bi-, or multivalent (i.e., having one, two, or multiple antigen binding domains).

In addition to whole antibodies, the present invention encompasses chimeric antibodies, hybrid antibodies, and fragments, such as scFv, sFv, F(ab′)2, Fab′, Fab and the like, including hybrid fragments. Thus, fragments of the antibodies that retain the ability to bind their specific antigens are provided. For example, fragments of antibodies which maintain histone cleavage product binding activity are included within the meaning of antibody or antigen binding fragment thereof. Such antibodies and fragments can be made and screened for specificity and activity by techniques known in the art (see ANTIBODIES: A LABORATORY MANUAL (Harlow et al. eds., 1988) which is hereby incorporated by reference in its entirety).

Monovalent antibodies can be generated by in vitro digestion of antibodies to produce fragments, i.e., Fab fragments, using routine techniques known in the art. For instance, digestion can be performed using papain as described in WO94/29348 to Landon, and ANTIBODIES: A LABORATORY MANUAL (Harlow et al. eds., 1988), which are hereby incorporated by reference in their entirety. Papain digestion of antibodies typically produces two identical antigen binding fragments, called Fab fragments, each with a single antigen binding site, and a residual Fc fragment. Pepsin treatment yields a fragment, called the F(ab′)2 fragment, that has two antigen combining sites and is still capable of cross-linking antigen. Methods for generating stable monovalent antibody fragments for therapeutic utility are further described in WO/2005063816 to Huang et al., which is hereby incorporated by reference in its entirety.

The Fab fragments produced by antibody digestion contain the constant domains of the light chain and the first constant domain of the heavy chain. Fab′ fragments differ from Fab fragments by the addition of a few residues at the carboxy terminus of the heavy chain domain including one or more cysteines from the antibody hinge region. The F(ab′)2 fragment is a bivalent fragment comprising two Fab′ fragments linked by a disulfide bridge at the hinge region. Other chemical couplings of antibody fragments are also known.

An isolated immunogenic specific paratope or fragment of the antibody is also provided. A specific immunogenic epitope of the antibody can be isolated from the whole antibody by chemical or mechanical disruption of the molecule. The purified fragments thus obtained are tested to determine their immunogenicity and specificity. Immunoreactive paratopes of the antibody, optionally, are synthesized directly. An immunoreactive fragment is defined as an amino acid sequence of at least about two to five consecutive amino acids derived from the antibody amino acid sequence.

The antibodies of the present invention can be generated in a non-human species and “humanized” for administration in humans. Humanized forms of non-human (e.g., murine) antibodies are chimeric immunoglobulins, immunoglobulin chains or fragments thereof which contain minimal sequence derived from non-human immunoglobulin. Humanized antibodies include human immunoglobulins in which residues of the complementary determining region (CDR) are replaced by residues from a CDR of a non-human species such as mouse, rat, or rabbit having the desired specificity, affinity, and capacity. In some instances, Fv framework residues of the human immunoglobulin are replaced by corresponding non-human residues. Methods for humanizing non-human antibodies are well known in the art as described in U.S. Pat. No. 4,816,567 to Cabilly et al.; Jones et al., “Replacing the Complementarity-Determining Regions in a Human Antibody with those From a Mouse,” Nature 321:522-525 (1986); Riechmann et al., “Reshaping Human Antibodies for Therapy,” Nature 332:323-327 (1988); and Verhoeyen et al., “Reshaping Human Antibodies:Grafting an Antilysozyme Activity,” Science 239:1534-1536 (1988), which are hereby incorporated by reference in their entirety.

Another aspect of the present invention relates to a method of diagnosing cancer in a subject, which involves providing a sample from the subject and contacting the sample with an antibody that selectively binds to a histone-3 cleavage product. The method further involves identifying the presence of a histone-3 cleavage product in the sample with the antibody that selectively binds to a histone-3 cleavage product and diagnosing cancer in the subject based on the identifying step.

An antibody directed to the C-terminus of the histone-3 cleavage product of SEQ ID NO:2 or the N-terminus of the histone-3 cleavage product of SEQ ID NO:3 can be used to identify the presence of a histone-3 cleavage product. A preferred antibody for use in the methods of the present invention is the H3.cs1 antibody described infra.

Identifying the presence of a histone-3 cleavage product in a sample from the subject using an antibody directed to a histone-3 cleavage product can be carried out using any standard immunochemical assay known in the art, including, but not limited to, ELISA, Western blot, immunocytochemistry, immunohistochemistry, or flow cytometry.

Another aspect of the present invention relates to a method of monitoring a subject's response to cancer treatment. This method involves obtaining a first biological sample from the subject before administration of a cancer treatment and a second biological sample from the subject after administration of the cancer treatment, and contacting the samples with an antibody that selectively binds to a histone-3 cleavage product. The presence of a histone-3 cleavage product in the samples is identified with the antibody and the subject's response to cancer treatment can be monitored based on the presence or absence of the histone-3 cleavage product.

In a preferred embodiment of this aspect of the present invention, the method is used to monitor a subject's response to cancer treatment, where the cancer treatment includes administration of an HDAC inhibitor. In this embodiment, the presence of a histone-3 cleavage product indicates the subject is not responsive to HDAC inhibitor therapy and the absence of a histone-3 cleavage product indicates the subject is responsive to HDAC inhibitor therapy.

An antibody directed to the C-terminus of the histone-3 cleavage product of SEQ ID NO:2 or the N-terminus of the histone-3 cleavage product of SEQ ID NO:3 can be used to identify the presence of a histone-3 cleavage product using any standard immunochemical assay known in the art. A preferred antibody for use in the methods of the present invention is the H3.cs1 antibody described infra.

The present invention is also directed to a method of identifying candidate compounds useful for modulating histone proteolysis. This method involves providing the candidate compound and a population of differentiating stem cells, and contacting the candidate compound and the population of differentiating stem cells under conditions effective for the candidate compound to modulate histone proteolysis. The presence or absence of a histone cleavage product in the population of differentiated stem cells is detected and a compound useful for modulating histone proteolysis is identified based the presence or absence of a cleavage product.

The candidate compound can be any chemical compound, for example, a small organic molecule, a carbohydrate, a lipid, an amino acid, a polypeptide, a nucleosides, a nucleic acid, or a peptide nucleic acid. The candidate compound can be naturally occurring, synthetic, or both.

In accordance with this aspect of the present invention, failure to detect a histone cleavage product in the differentiating stem cells identifies a compound useful for inhibiting histone proteolysis. The identified compound may be a cathepsin L or HDAC inhibitor. Alternatively, the identified compound may inhibit histone proteolysis by another mechanism.

The above method can further involve contacting the candidate compound with the population of differentiating stem cells in the presence of a cathepsin L inhibitor or an HDAC inhibitor. Under these conditions, detecting the presence of a histone-3 cleavage product identifies a compound useful for inducing histone proteolysis.

Detection of the histone cleavage product can be carried out using any of the antibodies of the present invention. In a preferred embodiment, the antibody selectively recognizes a histone-3 cleavage product. More preferably, the antibody selectively recognizes the C-terminus of the histone-3 cleavage product of SEQ ID NO:2 or the N-terminus of the histone-3 cleavage product of SEQ ID NO:3.

An additional aspect of the present invention relates to a method of treating a subject having cancer. This method involves selecting a patient based on his/her propensity to undergo histone proteolysis at a KQLATK motif (SEQ ID NO:4) and administering an agent that modulates histone proteolysis to the subject under conditions effective to treat cancer.

In accordance with this aspect of the present invention, a patient having a propensity to undergo histone proteolysis at a KQLATK motif is selected by identifying the presence of a histone-3 cleavage product in a sample from the subject. The presence of a histone 3 cleavage product in a sample from the subject can be identified using the antibodies of the present invention that are directed to a histone-3 cleavage product using standard immunochemical assays in the art (i.e., ELISA, Western blot, immunocytochemistry, immunohistochemistry, or flow cytometry).

Once a patient's propensity to undergo histone proteolysis at a KQLATK motif is identified, an appropriate agent that modulates histone proteolysis is administered to the subject under conditions effective to treat cancer. Accordingly, in one embodiment of this aspect of the invention, it may be preferable to administer an agent that inhibits proteolysis of histone-3. Agents that inhibit histone proteolysis, in particular histone-3 proteolysis, include cathepsin inhibitors and histone deacetylase inhibitors. Any of the above described cathepsin, in particular, cathepsin L inhibitors can be administered to the subject having cancer. Likewise, any of the above described histone deacetylase inhibitors can also be administered to a patient.

In an alternative embodiment of this aspect of the invention, it is preferable to administer an agent that induces proteolysis of the histone-3. Agents that induce histone proteolysis, in particular histone-3 proteolysis, include agents that induce, enhance, or mimic cathepsin L activity, recombinant cathepsin L proteins or proteolytic active cathepsin L polypeptides, or nucleic acid molecules encoding the same, or a histone acetyltransferase inhibitor. Any of the cathepsin inducers or activators, recombinant proteins or nucleic acid molecules, or histone acetyltransferase inhibitors described supra are suitable for administration to a subject to treat cancer according to this aspect of the present invention.

In accordance with this aspect of the present invention, the agents suitable for administration to a subject having cancer are in the form of a pharmaceutical composition. Appropriate pharmaceutical compositions containing the agents suitable for use in the present invention may vary depending on the route of administration and mode of delivery.

Agents of the invention that are nucleic acids molecules, such as siRNA or antisense cathepsin L inhibitors, or nucleic acids encoding cathepsin L recombinant proteins or proteolytic polypeptides, may be delivered using suitable gene therapy approaches and compositions. Methods of introducing nucleic acid molecule encoding a polypeptide of interest into expression vectors, including those vectors suitable for gene therapy delivery are described supra. Gene therapy vectors can be delivered to a subject by, for example, intravenous injection, local administration (see U.S. Pat. No. 5,328,470 to Nabel et al., which is hereby incorporated by reference in its entirety) or by stereotactic injection (see e.g., Chen et al. “Gene Therapy for Brain Tumors Regression of Experimental Gliomas by Adenovirus Mediated Gene Transfer In Vivo,” Proc. Nat'l. Acad. Sci. 91:3054-3057 (1994), which is hereby incorporated by reference in its entirety). The pharmaceutical preparation of the gene therapy vector can include the gene therapy vector in an acceptable diluent, or a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system. The pharmaceutical compositions can be included in a container. Gene therapy vectors typically utilize constitutive regulatory elements which are responsive to endogenous transcriptions factors. In a preferred embodiment, the gene therapy vectors encoding the cathepsin L recombinant protein or active peptide is an expression vector derived from a virus that is an adenovirus, adeno-associated virus, retrovirus, lentivirus, or herpes virus. Adenoviral viral vector gene delivery vehicles can be readily prepared and utilized given the disclosure provided in Berkner, “Development of Adenovirus Vectors for the Expression of Heterologous Genes,” Biotechniques 6:616-627 (1988) and Rosenfeld et al., “Adenovirus-Mediated Transfer of a Recombinant Alpha 1-Antitrypsin Gene to the Lung Epithelium In Vivo,” Science 252:431-434 (1991), WO 93/07283 to Curiel et al., WO 93/06223 to Perricaudet et al., and WO 93/07282 to Curiel et al., which are hereby incorporated by reference in their entirety. Adeno-associated viral gene delivery vehicles can be constructed and used to deliver a gene to cells as described in Chatterjee et al., “Dual-Target Inhibition of HIV-1 In Vitro by Means of an Adeno-Associated Virus Antisense Vector,” Science 258:1485-1488 (1992); Walsh et al., “Regulated High Level Expression of a Human Gamma-Globin Gene Introduced Into Erythroid Cells by an Adeno-Associated Virus Vector,” Proc. Nat'l. Acad. Sci. 89:7257-7261 (1992); Walsh et al., “Phenotypic Correction of Fanconi Anemia in Human Hematopoietic Cells With a Recombinant Adeno-Associated Virus Vector,” J. Clin Invest. 94:1440-1448 (1994); Flotte et al., “Expression of the Cystic Fibrosis Transmembrane Conductance Regulator From a Novel Adeno-Associated Virus Promoter,” J. Biol. Chem. 268:3781-3790 (1993); Ponnazhagan et al., “Suppression of Human Alpha-Globin Gene Expression Mediated by the Recombinant Adeno-Associated Virus 2-Based Antisense Vectors,” J. Exp. Med. 179:733-738 (1994); and Zhou et al., “Adeno-associated Virus 2-Mediated Transduction and Erythroid Cell-Specific Expression of a Human Beta-Globin Gene,” Gene Ther. 3:223-229 (1996), which are hereby incorporated by reference in their entirety. In vivo use of these vehicles is described in Flotte et al., “Stable in Vivo Expression of the Cystic Fibrosis Transmembrane Conductance Regulator With an Adeno-Associated Virus Vector,” Proc. Nat'l Acad. Sci. 90:10613-10617 (1993); and Kaplitt et al., “Long-Term Gene Expression and Phenotypic Correction Using Adeno-Associated Virus Vectors in the Mammalian Brain,” Nature Genet. 8:148-153 (1994), which are hereby incorporated by reference in their entirety. Additional types of adenovirus vectors are described in U.S. Pat. No. 6,057,155 to Wickham et al.; U.S. Pat. No. 6,033,908 to Bout et al.; U.S. Pat. No. 6,001,557 to Wilson et al.; U.S. Pat. No. 5,994,132 to Chamberlain et al.; U.S. Pat. No. 5,981,225 to Kochanek et al.; U.S. Pat. No. 5,885,808 to Spooner et al.; and U.S. Pat. No. 5,871,727 to Curiel, which are hereby incorporated by reference in their entirety.

Retroviral vectors which have been modified to form infective transformation systems can also be used to deliver a nucleic acid encoding a desired protein or polypeptide into a target cell. One such type of retroviral vector is disclosed in U.S. Pat. No. 5,849,586 to Kriegler et al., which is hereby incorporated by reference.

Regardless of the type of infective transformation system employed, it should be targeted for delivery of the nucleic acid to a specific cell type. For example, for delivery of the nucleic acid into tumor cells, a high titer of the infective transformation system can be injected directly within the tumor site so as to enhance the likelihood of tumor cell infection. The infected cells will then express the desired protein product, for example a cathepsin L recombinant protein or active peptide fragment, to immolate the cancer cell.

Non-viral gene delivery vehicles are also a means to effect cell-specific delivery of the therapeutic plasmids for the present invention. These are traditionally antibodies or single-chain Fv antibodies that are coupled or fused to DNA complexing agents (See Uherek et al., “Long-Term Gene Expression and Phenotypic Correction Using Adeno-Associated Virus Vectors in the Mammalian Brain,” J. Biol. Chem. 273:8835-8841 (1998); Foster et al., “HER2—Targeted Gene Transfer,” Human Gene Ther. 8:719-727 (1997); Chen et al., “Design of a Genetic Immunotoxin to Eliminate Toxin Immunogenicity,” Gene Ther. 2:116-123 (1995), which are hereby incorporated by reference in their entirety). This class of gene delivery vehicles also includes antibodies or their fragments coupled to liposomes (U.S. Pat. Nos. 4,925,661, 4,957,735, and 6,008,202 to Huang, which are hereby incorporated by reference in their entirety).

Another approach for delivering recombinant proteins or peptides, siRNA molecules or other nucleic acid molecules, cathepsin L inhibitors, and histone acetylase and deacetylase inhibitors of the present invention directly into cells involves the use of liposomes. Liposomes are vesicles comprised of one or more concentrically ordered lipid bilayers which encapsulate an aqueous phase. They are normally not leaky, but becomes leaky if a hole or pore occurs in the membrane, if the membrane is dissolved or degrades, or if the membrane temperature is increased to the phase transition temperature. Current methods of drug delivery via liposomes require that the liposome carrier ultimately become permeable and release the encapsulated drug, in this case recombinant cathepsin L protein or active peptide fragment, siRNA molecule, cathepsin L inhibitor, or HAT or HDAC inhibitor at the target site. This can be accomplished, for example, in a passive manner wherein the liposome bilayer degrades over time through the action of various agents in the body. Every liposome composition will have a characteristic half-life in the circulation or at other sites in the body and, thus, by controlling the half-life of the liposome composition, the rate at which the bilayer degrades can be regulated.

In contrast to passive drug release, active drug release involves using an agent to induce a permeability change in the liposome vesicle. Liposome membranes can be constructed so that they become destabilized when the environment becomes acidic near the liposome membrane (see e.g., Wang et al., “pH-Sensitive Immunoliposomes Mediate Target-Cell-Specific Delivery and Controlled Expression of a Foreign Gene in Mouse,” Proc. Natl. Acad. Sci. USA 84:7851 (1987), which is hereby incorporated by reference). When liposomes are endocytosed by a target cell, for example, they can be routed to acidic endosomes which will destabilize the liposome and result in drug release. Alternatively, the liposome membrane can be chemically modified such that an enzyme is placed as a coating on the membrane which slowly destabilizes the liposome. The liposome delivery system can also be made to accumulate at a target organ, tissue, or cell via active targeting (e.g., by incorporating an antibody or hormone on the surface of the liposomal vehicle). This can be achieved according to known methods.

Different types of liposomes can be prepared according to Bangham et al., “Diffusion of Univalent Ions Across the Lamellae of Swollen Phospholipids,” J. Mol. Biol. 13:238-252 (1965); U.S. Pat. No. 5,653,996 to Hsu et al.; U.S. Pat. No. 5,643,599 to Lee et al.; U.S. Pat. No. 5,885,613 to Holland et al.; U.S. Pat. No. 5,631,237 to Dzau et al.; and U.S. Pat. No. 5,059,421 to Loughrey et al., which are hereby incorporated by reference in their entirety.

An alternative approach for delivery of proteins or polypeptides involves the conjugation of the desired protein or polypeptide to a polymer that is stabilized to avoid enzymatic degradation of the conjugated protein or polypeptide. Conjugated proteins or polypeptides of this type are described in U.S. Pat. No. 5,681,811 to Ekwuribe, which is hereby incorporated by reference in its entirety.

Yet another approach for delivery of proteins or polypeptides involves preparation of chimeric proteins according to U.S. Pat. No. 5,817,789 to Heartlein et al., which is hereby incorporated by reference in its entirety. A chimeric protein suitable for use in the methods of the present invention contains a ligand binding domain and the recombinant cathepsin L protein or active peptide thereof or a peptide cathepsin L inhibitor. The ligand binding domain is specific for cell surface receptors located on a target cell. Thus, when the chimeric protein is delivered intravenously or otherwise introduced into blood or lymph, the chimeric protein will be selectively taken up and internalized by the target cell.

In practicing the method of the present invention, agents suitable for treating a subject having cancer can be administered using any method standard in the art. The agents, in their appropriate delivery form, can be administered topically, enterally (i.e., orally, bucally, rectally), parenterally (i.e., intradermally, intramuscularly, intraperitoneally, intravenously, subcutaneously), or intranasally. The compositions of the present invention may be administered alone or with suitable pharmaceutical carriers, and can be in solid or liquid form, such as tablets, capsules, powders, solutions, suspensions, or emulsions.

The agents of the present invention may be orally administered, for example, with an inert diluent, or with an assimilable edible carrier, or it may be enclosed in hard or soft shell capsules, or it may be compressed into tablets, or they may be incorporated directly with the food of the diet. Agents of the present invention may also be administered in a time release manner incorporated within such devices as time-release capsules or nanotubes. Such devices afford flexibility relative to time and dosage. For oral therapeutic administration, the agents of the present invention may be incorporated with excipients and used in the form of tablets, capsules, elixirs, suspensions, syrups, and the like. Such compositions and preparations should contain at least 0.1% of the agent, although lower concentrations may be effective and indeed optimal. The percentage of the agent in these compositions may, of course, be varied and may conveniently be between about 2% to about 60% of the weight of the unit. The amount of an agent of the present invention in such therapeutically useful compositions is such that a suitable dosage will be obtained.

Also specifically contemplated are oral dosage forms of the agents of the present invention. The agents may be chemically modified so that oral delivery of the derivative is efficacious. Generally, the chemical modification contemplated is the attachment of at least one moiety to the component molecule itself, where said moiety permits (a) inhibition of proteolysis; and (b) uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline (Abuchowski and Davis, “Soluble Polymer-Enzyme Adducts,” in ENZYMES AS DRUGS 367-383 (Hocenberg and Roberts eds., 1981), which is hereby incorporated by reference in its entirety). Other polymers that could be used are poly-1,3-dioxolane and poly-1,3,6-tioxocane. Preferred for pharmaceutical usage, as indicated above, are polyethylene glycol moieties.

The tablets, capsules, and the like may also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, sucrulose, or saccharin. When the dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar, or both. A syrup may contain, in addition to active ingredient, sucrose as a sweetening agent, methyl and propylparabens as preservatives, a dye, and flavoring such as cherry or orange flavor.

The agents of the present invention may also be administered parenterally. Solutions or suspensions of the agent can be prepared in water suitably mixed with a surfactant such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions. Under ordinary conditions of storage and use, these preparations contain a preservative to prevent the growth of microorganisms.

Pharmaceutical formulations suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

When it is desirable to deliver the agents of the present invention systemically, they may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Intraperitoneal or intrathecal administration of the agents of the present invention can also be achieved using infusion pump devices such as those described by Medtronic, Northridge, Calif. Such devices allow continuous infusion of desired compounds avoiding multiple injections and multiple manipulations.

In addition to the formulations described previously, the agents may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt

The agents of the present invention may also be administered directly to the airways in the form of an aerosol. This form of administration is particularly suited for siRNA delivery. For use as aerosols, the agent of the present invention in solution or suspension may be packaged in a pressurized aerosol container together with suitable propellants, for example, hydrocarbon propellants like propane, butane, or isobutane with conventional adjuvants. The agent of the present invention also may be administered in a non-pressurized form such as in a nebulizer or atomizer.

Effective doses of the compositions of the present invention, for the treatment of cancer vary depending upon many different factors, including type and stage of cancer, means of administration, target site, physiological state of the patient, other medications or therapies administered, and physical state of the patient relative to other medical complications. Treatment dosages need to be titrated to optimize safety and efficacy.

### EXAMPLES

The Examples set forth below are for illustrative purposes only and are not intended to limit, in any way, the scope of the present invention.

### Example 1

### Cell Culture and Differentiation

ES cells (LF2 line) were cultured as previously described (Bernstein et al., “Mouse Polycomb Proteins Bind Differentially to Methylated Histone H3 and RNA and are Enriched in Facultative Heterochromatin,” Mol Cell Biol 26:2560-2569 (2006), which is hereby incorporated by reference in its entirety). Cells were grown on gelatin-coated plates without feeder cells and maintained in an undifferentiated state through culture in KODMEM (Invitrogen 1082-9018)), 2 mM L-glutamine (Sigma G7513), 15% ES grade fetal bovine serum (Gibco 10439-024), 10⁻⁴ mM 2-mercaptoethanol, and leukemia inhibitory factor (LIF). To differentiate, cells were plated sparsely at ˜1*10⁴ cells/cm², allowed to adhere overnight and then fed with differentiation media containing DMEM, 10% FBS, 10⁻⁴ mM 2-mercaptoethanol and, for retinoic acid (RA) differentiation, 100 nM all-trans-RA (Sigma R2625). Embryoid body formation was accomplished by splitting partially trypsinized cells onto non-treated petri dishes and allowing cells to cluster. Chemical inhibition of Cathepsin L was accomplished by adding 10 uM [final] Cathepsin L I inhibitor (Calbiochem, Cat. No. 219402) to the cell media.

### Example 2

### Cell Extract Preparation

Whole cell extracts were prepared by resuspending fresh or flash frozen cell pellets in SDS-Laemmli sample buffer and boiling immediately. Chromatin extracts were prepared by sonicating chromatin pellets in buffer (10 mM HEPES, 10 mM KCl, 1.5 mM MgCl₂, 0.34M sucrose, 10% glycerol, 5 mM β-mercaptoethanol) after isolation by various methods (high salt extraction as described by Dignam et al., “Accurate Transcription Initiation by RNA Polymerase II in a Soluble Extract From Isolated Mammalian Nuclei,” Nucleic Acids Res 11:1475-1489 (1983), which is hereby incorporated by reference in its entirety, or chromatin fractionation as described by Mendez et al., “Chromatin Association of Human Origin Recognition Complex, cdc6, and Minichromosome Maintenance Proteins During the Cell Cycle: Assembly of Prereplication Complexes in Late Mitosis,” Mol Cell Biol 20:8602-8612 (2000), which is hereby incorporated by reference in its entirety). Briefly, cells were swelled in low salt buffer and then lysed by either mechanical disruption (Dignam method) or detergent (Stillman method); soluble nuclear proteins were then either extracted with high-salt (Dignam) or released by incubation in no salt buffer and mechanical disruption (Stillman), leaving behind the chromatin pellet. Protease inhibitors were omitted from extract preparations used for activity assays. Digestion of chromatin fractions into mononucleosomes was accomplished by treatment with microccocal nuclease as described (Wysocka et al., “Loss of HCF-1-Chromatin Association Precedes Temperature-Induced Growth Arrest of tsBN67 Cells,” Mol Cell Biol 21:3820-3829 (2001), which is hereby incorporated by reference in its entirety).

### Example 3

### Histone Purification, Edman Degradation and MS-MS Mapping of H3Cleavage Sites

Histones were acid extracted from nuclei and purified by RP-HPLC using a C8 column as described (Shechter et al., “Extraction, Purification and Analysis of Histones,” Nat Protoc 2:1445-1457 (2007), which is hereby incorporated by reference in its entirety). RP-HPLC fractions containing the H3 sub-band were pooled and repurified by RP-HPLC using a C18 column. Equal amounts of fractions 52-55 were pooled, separated by SDS-PAGE, and blotted onto 0.2 μm pore size membrane (Millipore Cat. No. ISEQ00010). H3 sub-bands were excised and subjected to Edman degradation as described previously (Strahl et al., “Methylation of Histone H3 at Lysine 4 is Highly Conserved and Correlates With Transcriptionally Active Nuclei in Tetrahymena,” Proc Natl Acad Sci USA 96:14967-14972 (1999), which is hereby incorporated by reference in its entirety). Fraction 54 was digested with endoproteinase GluC (Roche Diagnostics, Indianapolis, Ind.) for 4 hours at 37° C. (1:20 wt:wt), and was loaded onto a C18 packed capillary column as previously described (Martin et al., “Subfemtomole MS and MS/MS Peptide Sequence Analysis Using Nano-HPLC Micro-ESI Fourier Transform Ion Cyclotron Resonance Mass Spectrometry,” Anal Chem 72:4266-4274 (2000), which is hereby incorporated by reference in its entirety). Samples were analyzed by nanoflow HPLC-microelectrospray ionization on a linear quadrupole ion trap-Fourier transform mass spectrometer (LTQ-FT; Thermo Electron) for accurate mass and a Thermo LTQ instrument modified for electron transfer dissociation (ETD) and proton transfer charge reduction (PTR) for adequate tandem mass spectrometry (MS/MS) as previously described (Coon et al., “Protein Identification Using Sequential Ion/Ion Reactions and Tandem Mass Spectrometry,” Proc Natl Acad Sci USA 102:9463-9468 (2005); Syka et al., “Peptide and Protein Sequence Analysis by Electron Transfer Dissociation Mass Spectrometry,” Proc Natl Acad Sci USA 101:9528-9533 (2004); and Syka et al., “Novel Linear Quadrupole Ion Trap/FT Mass Spectrometer: Performance Characterization and Use in the Comparative Analysis of Histone H3 Post-Translational Modifications,” J Proteome Res 3:621-626 (2004), which are hereby incorporated by reference in their entirety). Data was manually interpreted, as well as searched against an H3 database using OMSSA (Geer et al., Open Mass Spectrometry Search Algorithm,” J Proteome Res 3:958-964 (2004), which is hereby incorporated by reference in its entirety).

### Example 4

### In vitro H3Cleavage Assay

Extracts were incubated in buffer (10 mM HEPES, 10 mM KCl, 1.5 mM MgCl₂, 0.34M sucrose, 10% glycerol, 5 mM β-mercaptoethanol, all final) with 0.1 μg/μl rH3-HIS (purified from E. coli) and incubated at 37° C. for 1-3 hours. Reactions were stopped by addition of 5×SDS sample buffer and boiling; results were analyzed by immunoblotting with HIS-HRP and/or H3.cs1 antibody. For MS analysis, reactions were quenched by addition of 0.1% (final) TFA. rCathepsin L (R&D Systems, # 1515-CY-010), rCathepsin B (R&D Systems Cat No. BAF965), and rCathepsin K (Calbiochem Cat. No. 342001) were purchased from commercial sources and tested in the assay with rH3-HIS.

### Example 5

### Enzyme Enrichment and Identification

Three days+RA differentiating ESC chromatin pellets were solubilized by sonication in buffer A (80 mM NaPO⁻⁴, 200 mM NaCl), injected onto a 2 or 5 mL hydroxyapatite column (BioRad), and fractionated with a 200 mM to 2M NaCl gradient. Each fraction was then assayed as described above. Both active and inactive fractions were reduced with 1 mM DTT at 51° C. for 1 hr and alkylated with 2 mM iodoacetamide in the dark at RT for 45 mins, followed by digestion with trypsin (Promega Corp., Madison, Wis.) at an enzyme to substrate ratio of 1:20 (wt:wt) for 6 hrs at 37° C. Digest was then acidified with glacial acetic acid and aliquots of samples were analyzed using an LTQ-FT as described above. Data was searched against a mouse database using SEQUEST (Eng et al., Journal of the American Society of Mass Spectrometry 5:976-989 (1994), which is hereby incorporated by reference in its entirety). All spectra of interest were manually validated.

### Example 6

### Antibodies

The following antibodies were purchased from commercial vendors:

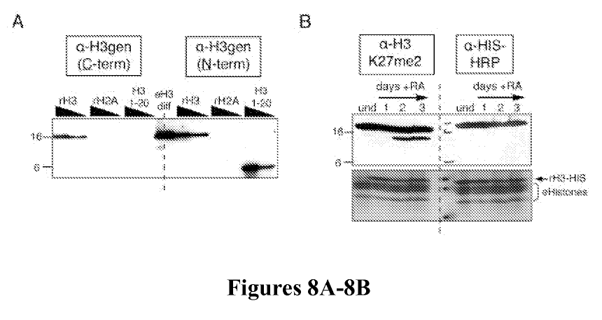

Cathepsin L (R&D Systems Cat. No. AF1515), Cathepsin B (R&D Systems Cat. No. AF965), Penta-HIS HRP Conjugate Kit (Qiagen Cat. No. 34460), Oct3/4 (BD Transduction Laboratories Cat. No. 611202), H3gen (Abcam Cat. No. 1791), H3K4me3 (Abcam Cat. No. 8580), H3K27me2 (Millipore Cat. No. 07-452). The cleavage-specific H3.cs1 antibody was generated as follows: a 2× branched peptide corresponding to mammalian histone H3 sequence 22-26 was conjugated to KLH and injected into rabbits (Covance). Serum was collected and tested for specificity as described in FIG. 11. The N-terminal H3gen antibody was generated as follows: a 6 amino acid peptide corresponding to mammalian histone H3 sequence 1-6 was conjugated to KLH and injected into rabbits (Covance). Serum was collected and recognition of the N-terminus was shown by recognition of an H3 1-20 peptide (compared to no recognition by the Abcam C-terminal H3gen antibody) as shown in FIG. 8.

### Example 7

### Plasmid Construction and Recombinant Protein Purification

A PCR fragment of the complete mouse histone H3 ORF was cloned into the pET30a plasmid vector such that a C-terminal HIS tag was added to the coding sequence. Mutations were made using the Quick-Change Mutagenesis II kit (Stratagene). Plasmids were transformed into BL21 E. coli and rH3-HIS protein was purified from inclusion bodies using both Ni²⁺-NTA and C8 columns (RP-HPLC). Acetic anhydride treatment was performed as described previously (Garcia et al., “Chemical Derivatization of Histones for Facilitated Analysis by Mass Spectrometry,” Nat Protoc 2:933-938 (2007), which is hereby incorporated by reference in its entirety). rH3-HIS was converted to rH3-HIS+K27me2 by first mutating K27 to C and C110 to A and then alkylating the cysteine to dimethyl by published methods (Simon et al., “The Site-Specific Installation of Methyl-Lysine Analogs into Recombinant Histones,” Cell 128:1003-1012 (2007), which is hereby incorporated by reference in its entirety). BPTF and CBX7 proteins were made as described in previous studies (Bernstein et al., “Mouse Polycomb Proteins Bind Differentially to Methylated Histone H3 and RNA and are Enriched in Facultative Heterochromatin,” Mol Cell Biol 26:2560-2569 (2006) and Li et al., “Molecular Basis for Site-Specific Read-Out of Histone H3K4me3 by the BPTF PHD Finger of NURF,” Nature 442:91-95 (2006), which are hereby incorporated by reference).

### Example 8

### RNA Isolation, Reverse Transcription and Q-PCR

RNA was isolated from undifferentiated and +RA differentiating ESCs using Trizol reagent as directed by the manufacturer (Invitrogen Cat. No. 15596-018). RNA was transcribed into cDNA using the First Strand Superscript kit (Invitrogen Cat. No. 18080-051) and analyzed by quantitative PCR using SYBRgreen reagent (Applied Biosystems Cat. No. 4309155).

### Example 9

### RNAi Knockdown

Short hairpins to both a control gene (a human gene that does not share sequence homology to the mouse genome) and the Ctsl gene were purchased from Open Biosystems (Cat. No. RHS3979-9628371 and RMM3981-9597987) and transfected into 293T cells to produce Lentiviral particles. The virus was then used to create ESC lines by infection and selection with puromycin. Heterogeneous cell populations expressing either the control or Ctsl RNAi were then differentiated with RA as usual.

### Example 10

### Peptide Pull-Downs

Peptide pull-down assays were performed as described Wysocka, J. “Identifying Novel Proteins Recognizing Histone Modifications Using Peptide Pull-down Assay,” Methods 40:339-343 (2006), which is hereby incorporated by reference in its entirety, using biotinylated peptides conjugated to streptavidin agarose beads (Pierce Cat. No. 20349). Elutions were loaded onto 10 or 15% SDS-PAGE gels and silver stained.

### Example 11

### A Faster Migrating H3 Species Is Detected in Differentiating Mouse ESCs

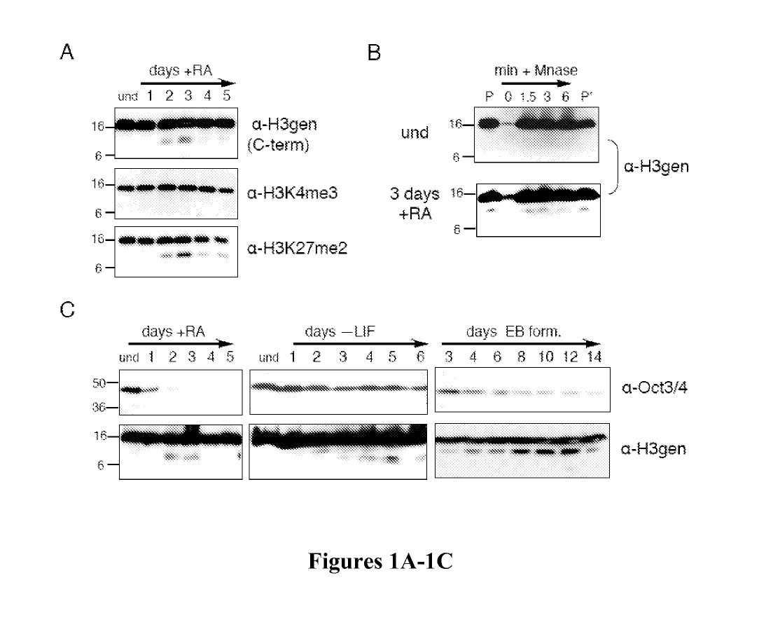

To survey changes in histone proteins and their modifications during mouse ESC differentiation, immunoblotting was used to probe whole-cell extracts (WCEs) with various histone antibodies. When probing with a specific subset of histone H3 antibodies (e.g., including the H3 general C-terminal, H3K27me2, and H3K27me1 antibodies), a faster migrating band at ˜14 kD was reproducibly observed in samples taken at time points corresponding to days two and three post-induction with retinoic acid (RA). Notably, this band(s) was observed using an H3 general antibody generated against the C-terminus of histone H3 (FIG. 1A, upper panel and FIG. 8A, left panel), but not with an H3 general antibody generated against the first six N-terminal amino acids (FIG. 8A, right panel). The faster migrating H3 species was also observed when probing immunoblots with an H3-K27me2 antibody (FIG. 1A, lower panel and FIG. 8B, left panel); in contrast, it was not recognized when replicate immunoblots were probed with the H3-K4me3 antibody (FIG. 1A, middle panel). Taken together, the results of these experiments suggested that an extreme amino-terminal fragment of H3 was missing in the faster-migrating H3 sub-band.

To determine whether the H3 sub-band was chromatin associated, micrococcal-digested chromatin was prepared by standard methods (Wysocka et al., “Loss of HCF-1-Chromatin Association Precedes Temperature-Induced Growth Arrest of tsBN67 Cells,” Mol Cell Biol 21:3820-3829 (2001), which is hereby incorporated by reference in its entirety) from both undifferentiated ESCs and ESCs undergoing differentiation with RA, and soluble mononucleosomes were probed with an H3 general antibody. As shown in FIG. 1B, the faster migrating H3 band was seen in the undigested chromatin pellet as well as in the solubilized mononucleosomes derived from differentiating cells (FIG. 1B, lower panel), but not in the chromatin isolated from undifferentiated ESCs (FIG. 1B, upper panel).

It was next determined whether the appearance of the faster migrating H3 species was dependent on the methods employed to trigger ESC differentiation and whether changing the timing and progression of differentiation would also change the time point at which the faster migrating H3 was detected. To address this question, ESCs were differentiated using three different methods: monolayer differentiation with RA, monolayer differentiation by withdrawal of leukemic inhibitory factor (LIF), and embryoid body formation (EB formation) by cell aggregation. As shown in FIG. 1C (upper panels), the time course for expression of pluripotency marker Oct3/4 differs for each of the induction methods employed, suggesting differences in the timing and progression of differentiation. Timing of the appearance of the faster migrating H3 band is also dependent on the method used to induce ESC differentiation (FIG. 1C, lower panels), suggesting that this event is also dependent on the progress of differentiation. The faster migrating H3 is observed at days two and three post RA induction during monolayer differentiation (FIG. 1A, upper panel, and FIG. 1C, lower left panel), but delayed until day five following withdrawal of LIF (FIG. 1C, lower middle panel), the latter correlating with the similarly delayed decrease of Oct3/4. During EB formation, the faster migrating H3 species appeared early and then peaked between days eight and twelve (FIG. 1C, lower right panel), suggesting a slower and more complex differentiation progression. Monolayer differentiation with RA was chosen for subsequent experiments in order to minimize differences in the timing and heterogeneity of differentiation.

### Example 12

### Histone H3, Marked by Both “Active” and “Silent” Modifications, is Proteolytically Cleaved in the N-terminal Tail During ESC Differentiation

To determine the nature of the faster migrating H3 sub-species, histones were extracted from differentiating ESC nuclei three days post RA induction and then separated by reverse phase high pressure liquid chromatography (RP-HPLC, C8 column). Fractions containing the H3 sub-band were pooled and further resolved by RP-HPLC (C18 column), and the resulting fractions were screened by immunoblot as shown in FIG. 2A (left panel). C18-RP-HPLC fractions containing the H3 sub-band were then subject to two separate sequencing methods. First, equal amounts of fractions 52-54 were pooled, separated by SDS-PAGE, and transferred to immunoblot; the material in each of the two sub-bands labeled by asterisks was then subjected to Edman degradation (FIG. 2A, right). Although multiple amino acids were released during each cycle for both samples, the observed data strongly suggested that the top and bottom sub-bands contained sequences derived from cleavage following residues A21 and K27, respectively, in the N-terminal tail of H3.

Second, to define in greater detail the peptide sequences and post-translational modifications present in the faster migrating H3 species, an additional sample from fraction 54 (FIG. 2A) was digested with GluC to produce N-terminal H3-fragments ending in E50 and the resulting peptides were then analyzed by MS. Spectra recorded with a linear ion trap-Fourier transform mass spectrometer detected six, highly-modified, truncated, N-terminal, histone H3-peptides beginning at residues T22, K23, A24, A25, K27, and S28 (FIG. 2B, right column). Relative abundances of the six peptides suggest that the preferred cleavage sites are C-terminal to A21 and K23. These results suggest that the primary H3 cleavage site is between amino acids 21 and 22 of the amino terminus (FIG. 2C, H3.cs1) and that the final cleavage site is between amino acids 27 and 28. Notably, the list of cleavage sites detected by MS contains the two sites detected by Edman degradation (asterisks).

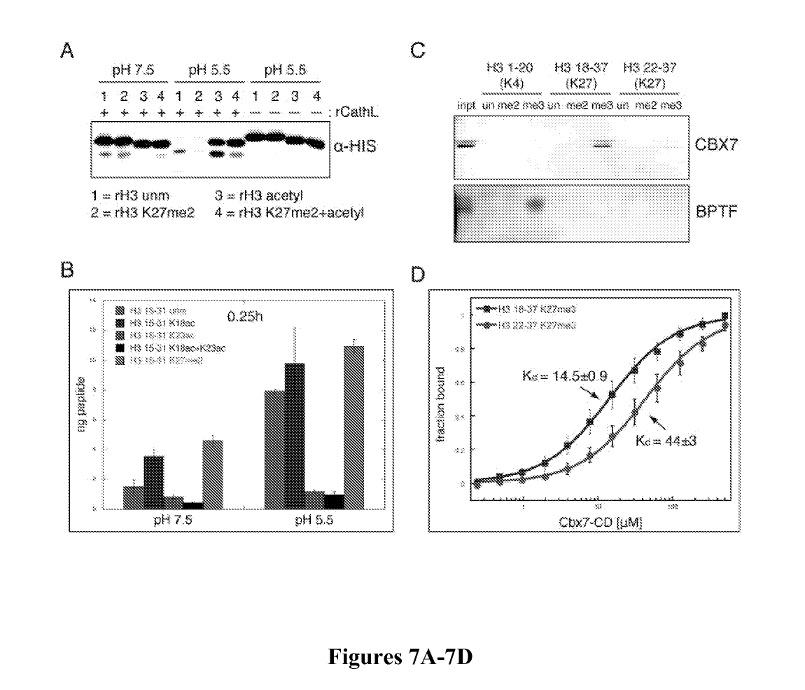

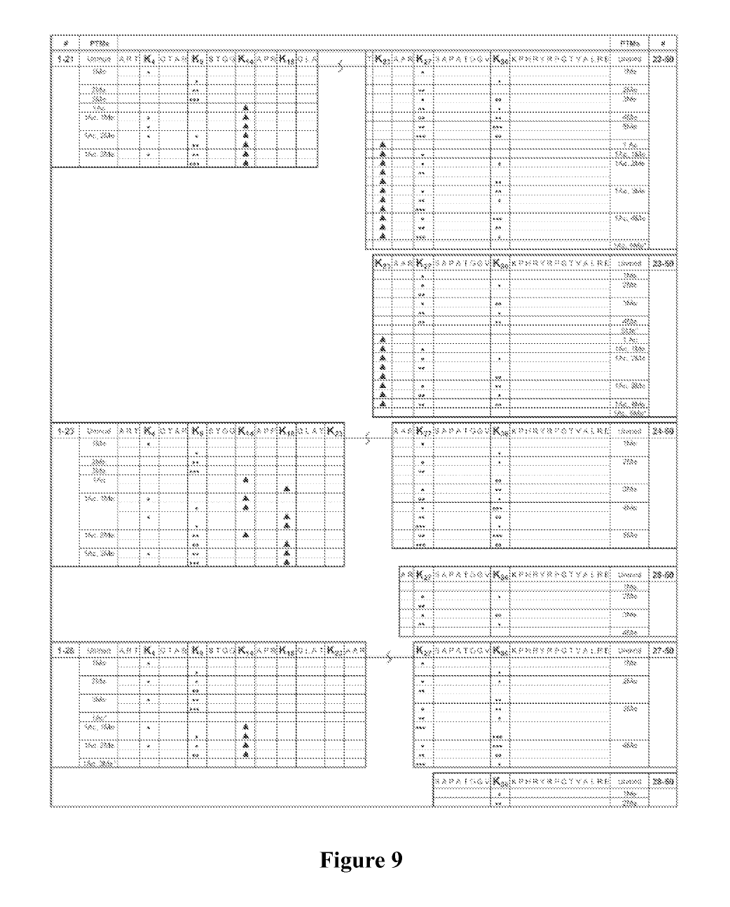

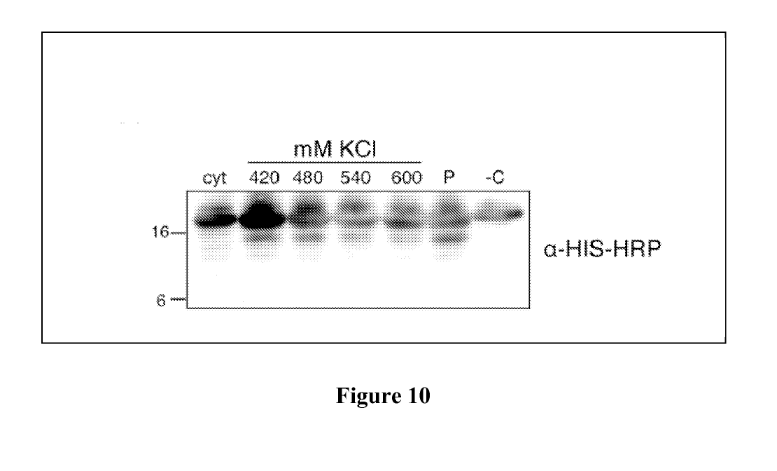

In addition, new mass spectrometric methods using a combination of electron transfer dissociation/proton transfer charge reduction and accurate mass measurements were employed to characterize the modification patterns on the proteolytically cleaved H3 (Coon et al., “Protein Identification Using Sequential Ion/Ion Reactions and Tandem Mass Spectrometry,” Proc Natl Acad Sci USA 102:9463-9468 (2005) and Taverna et al., “Long-Distance Combinatorial Linkage Between Methylation and Acetylation on Histone H₃N Termini,” Proc Natl Acad Sci USA 104:2086-2091 (2007), which is hereby incorporated by reference in its entirety). Interestingly, the data reveals that the cleaved H3 sub-species has a distinct covalent modification profile, suggesting that the H3 sub-band may be preferentially marked, before or after proteolytic processing, with a specific epigenetic signature (FIG. 2C and FIG. 9). Specifically, marks of both “active” (e.g., H3K23Ac and H3K36me) and “silent” transcription (e.g., H3K27me) were reproducibly detected on a single GluC-digested peptide derived from the proteolytically-processed H3 fragment. Moreover, all six of the truncated peptides contained Ala at position 31, revealing that the cleaved H3 peptide is H3 isoform H3.2, not H3.3. Although non-cleaved H3.3 peptide was detected in the same RP-HPLC fraction as the cleaved H3 species, cleaved H3.3 was not detected. Since H3.2 and H3.1 elute in two separate peaks, it cannot be definitively concluded that H3.2 is preferentially cleaved over H3.1, however significantly less H3 cleavage was detected in peak 2 (H3.1) than in peak 1 (H3.2+H3.3) by immunoblotting. Although it is unclear how this particular pattern of modifications and H3 isoform affect the mechanism of proteolysis, these data suggest the intriguing possibility that that regulation via post-translational modification of the substrate and/or isoform preference may regulate the proteolytic processing (see FIGS. 7A-7D).

Also detected in fraction 54 (FIG. 2A) were highly modified forms for three of the most abundant, complementary, N-terminal fragments generated by proteolytic cleavage of H3 (FIG. 2B, left column). The detection of these intact, complementary cleavage products indicates that the cleavage sites mapped above are the result of endopeptidase activity alone. Post-translational modifications detected on these three N-terminal peptides again include marks of both “active” (e.g., H3K14Ac) and “silent” transcription (e.g., H3K9me) as seen on the C-terminal fragments (FIG. 9). These findings support the conclusion that a small fraction of total histone H3.2 undergoes highly specific endoproteolytic cleavage during ESC differentiation that may be regulated by unique patterns of covalent modifications.

### Example 13

### The Lysosomal Cysteine Protease Cathepsin L is Present in Fractions That Are Enriched with H3 Protease Activity

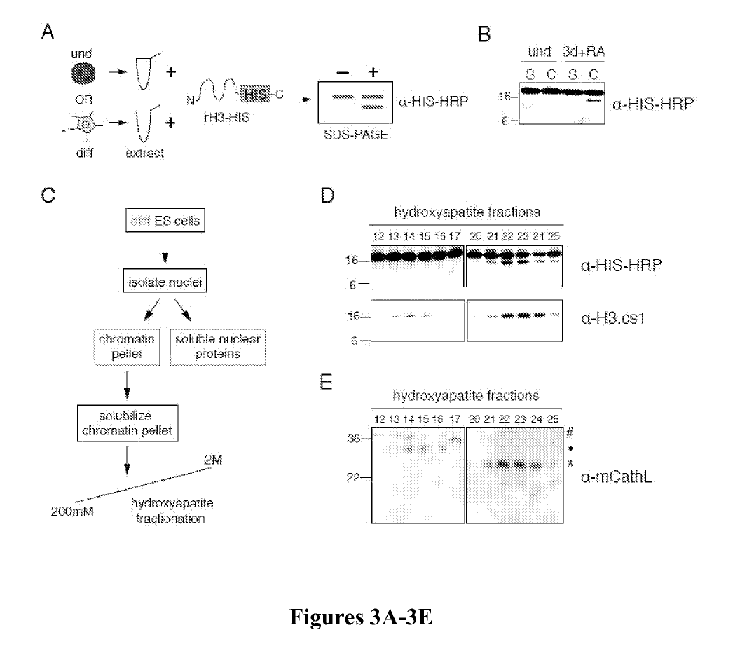

To identify and characterize the putative H3 protease(s), an in vitro H3 cleavage assay was established. Undifferentiated or differentiating ESCs were harvested and their proteins extracted. Cell lysates were then incubated with C-terminally 6×HIS tagged, full-length recombinant histone H3 (rH3-HIS), and the reaction products were analyzed by immunoblotting with HIS-HRP antibody (as depicted by the schematic in FIG. 3A). Although this assay has the potential to detect any N-terminal H3 cleavage, chromatin isolated from differentiating ESCs at the time point when in vivo cleavage was observed (˜3 days+RA) consistently possessed strong in vitro H3 cleavage activity (FIG. 3B). Preliminary experiments indicated that the in vitro H3 protease activity was associated with chromatin prepared by a variety of enrichment methods (Dignam et al., “Accurate Transcription Initiation by RNA Polymerase II in a Soluble Extract From Isolated Mammalian Nuclei,” Nucleic Acids Res 11:1475-1489 (1983); Hsieh et al., “Taspase1: A Threonine Aspartase Required for Cleavage of MLL and Proper HOX Gene Expression,” Cell 115:293-303 (2003); and Mendez et al., “Chromatin Association of Human Origin Recognition Complex, cdc6, and Minichromosome Maintenance Proteins During the Cell Cycle Assembly of Prereplication Complexes in Late Mitosis,” Mol Cell Biol 20:8602-8612 (2000), which are hereby incorporated by reference in their entirety) and produced a faster migrating H3 sub-band that reproduced the size difference of the endogenously cleaved H3.

To better ensure that the cleaved H3 product observed in the in vitro H3 cleavage assay was physiologically relevant, an antibody was generated that would recognize the new amino terminus created by the H3 protease. A 2× branched peptide antigen was synthesized that included amino acids 22-26 of H3 purposely leaving the amino terminus of T22 free such that it mimicked the primary site of H3 cleavage, H3.cs1 (FIG. 2C and FIG. 11A). The results indicated that this antibody, hereafter referred to as the H3.cs1 antibody, was specific for the primary site of in vivo generated H3 cleavage product (A21/T22) and was not sensitive to the acetylation status of H3K23 (see FIG. 11C). Importantly, the H3.cs1 antibody fails to react with full length H3 and its signal is highly enriched at the same time point at which an H3 sub-band was detected using the H3-general or H3 K27me2 antibodies during the standard RA-induced ESC differentiation time course (FIG. 11B).

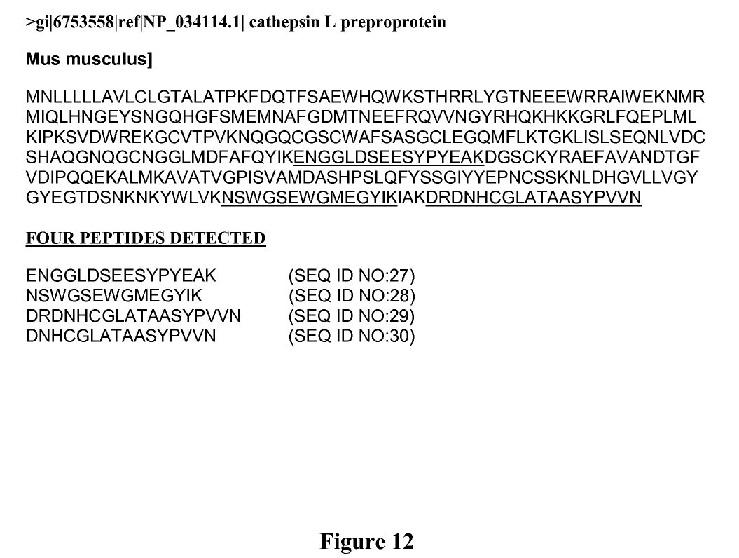

Both the HIS-HRP antibody and the H3.cs1 antibody were used in the above H3 cleavage assay to follow the biochemical enrichment of H3 protease activity of nuclear extracts derived from differentiating ESCs. Using the fractionation scheme depicted in FIG. 3C, two peaks of putative H3 cleavage activity were detected not only by the HIS-HRP antibody (FIG. 3D, upper panel), but also by the H3.cs1 antibody (FIG. 3D, lower panel). To identify a putative H3 protease in these fractions, two activity-containing fractions (#22 and 23, FIG. 3D) and an adjacent non-activity-containing fraction (#20) were subject to MS analysis. Interestingly, four peptides were detected for the cysteine protease Cathepsin L in both activity-containing fractions 22 and 23, yet none of these peptides were detected in the neighboring non-activity containing fraction 20 (see FIG. 12). To validate this identification further, a commercially available antibody was used to screen for the presence of Cathepsin L in the hydroxyapatite fractions (mCathL, FIG. 3E). Importantly, reactivity with mCathL correlates well with the detection of H3 cleavage activity in the fractions aligned above (compares FIGS. 3D and 3E).

Cathepsin L is known to exist in three principal processing forms: a proenzyme running at ˜37 kD, a single chain intermediate at ˜30 kD, and a double chain mature form at ˜25 kD and ˜5 kD (Ishidoh et al., “Multiple Processing of Procathepsin L to Cathepsin L In Vivo,” Biochem Biophys Res Commun 252:202-207 (1998), which is hereby incorporated by reference in its entirety). The pro form must be cleaved to become active, whether by self-cleavage or by another enzyme (Turk et al., “Lysosomal Cysteine Proteases: More than Scavengers,” Biochim Biophys Acta 1477:98-111 (2000), which is hereby incorporated by reference in its entirety); in contrast, both the “intermediate” and “mature” forms have been shown to be active (Mason et al., “The Identification of Active Forms of Cysteine Proteinases in Kirsten-Virus-Transformed Mouse Fibroblasts by Use of a Specific Radiolabelled Inhibitor,” Biochem J257:125-129 (1989), which is hereby incorporated by reference in its entirety). The hydroxyapatite fractions of the stronger peak of activity (˜21-25) correlate with the detection of the mature form of Cathepsin L by immunoblot (FIG. 3E, marked by asterisk), while the weaker activity fractions (˜13-15) correlate closely with the detection of the intermediate form of Cathepsin L (FIG. 3E, marked by dot). This correlation not only supports a causal relationship between the presence of Cathepsin L and the H3 cleavage activity, but also helps to explain the difference in elution and activity between the two peaks of H3 cleavage activity generated by the hydroxyapatite chromatography.

### Example 14

### Fractions Enriched in H3Cleavage Activity Exhibit Cathepsin L-like Activity

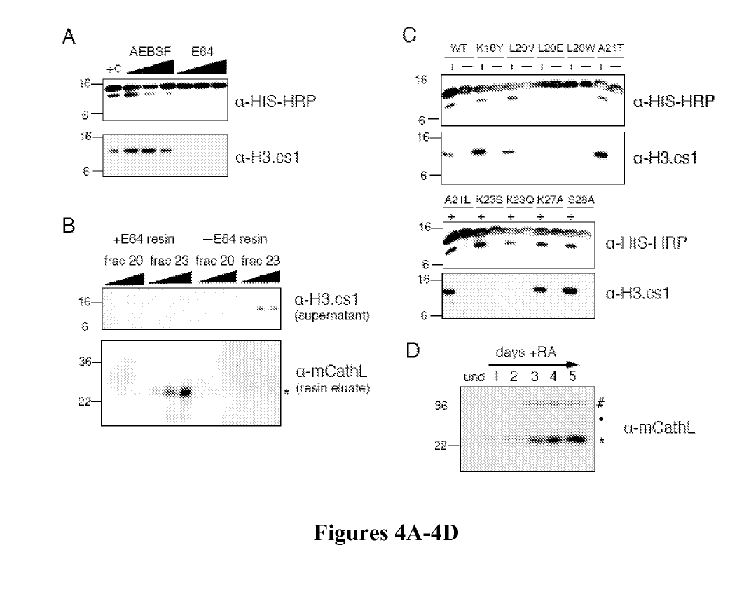

To validate further the identification of Cathepsin L as a histone H3 protease, the enriched fractions were tested with different classes of protease inhibitors in the H3 cleavage assay. As shown in FIG. 4A, adding increasing amounts of serine protease inhibitor AEBSF produced a modest reduction in cleavage activity at a high concentration (20 mM), but not at the lower concentrations tested (2 mM and 10 mM). However, when using the cysteine protease inhibitor E64, all cleavage was abolished at even the lowest concentration tested (10 μM).

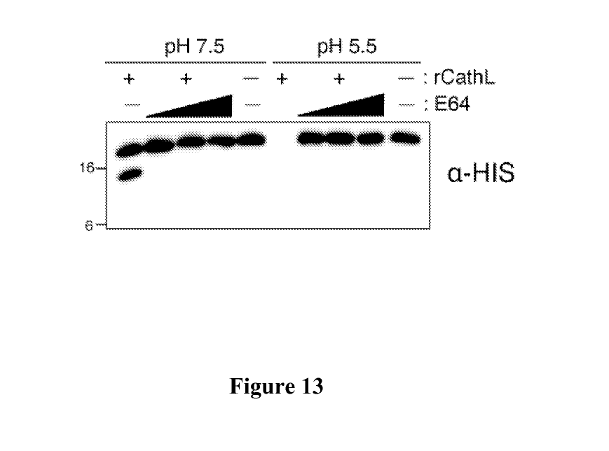

Noting the strong inhibition of H3 cleavage activity by E64, an irreversible inhibitor that binds covalently to its substrate and has been shown to inhibit Cathepsin L (See FIG. 13) (Barrett et al., “L-trans-Epoxysuccinyl-leucylamido(4-guanidino)butane (E-64) and its Analogues as Inhibitors of Cysteine Proteinases Including Cathepsins B, H and L,” Biochem J201:189-198 (1982), which is hereby incorporated by reference in its entirety), a commercially available E64-bound resin was utilized to test whether it could precipitate Cathepsin L from the active hydroxyapatite fractions and, subsequently, remove the soluble H3 cleavage activity. Both E64 bound and control resins were added to fractions 20 (in which little to no H3 cleavage activity detected) and 23 (in which strong H3 cleavage activity was detected), incubated to allow binding and potential inhibition, and then pelleted by centrifugation. The cleared supernatant was subsequently tested for H3 cleavage activity. As expected, E64 resin successfully removed H3 cleavage activity from fraction 23, while control resin did not (FIG. 4B, upper panel). Importantly, as shown using the cleavage-site specific H3.cs1 antibody, the activity removed by the E64 resin included the specific A21/T22 cleavage site activity. In contrast, the specific H3 cleavage activity remained in the supernatant of fraction 23 incubated with control resin (FIG. 4B, upper panel).

Any bound proteins were then eluted from the E64 and control resins by boiling in SDS sample buffer and the eluates were assayed for the presence of Cathepsin L by probing an immunoblot with mCathL antibody. As hypothesized, mCathL was detected on the +E64 resin that had been incubated with fraction 23, but was not found on control resin nor on resin incubated with fraction 20 (FIG. 4B, lower panel). As a control, the above eluates were analyzed by immunoblotting with an antibody to another cathepsin family member, Cathepsin B, but did not detect any immunoreactive species. Taken together, the loss of activity from solution paired with the presence of Cathepsin L protein bound to the corresponding +E64 resin strongly suggests a causal relationship between this cysteine protease and the primary H3 protease activity characterized above (cleavage of H3 between A21/T22, H3.cs1).

Cathepsin L is also known to preferentially cleave proteins that contain hydrophobic residues in their P2 position (two residues N-terminal from the cleaved bond, as originally defined (Schechter et al., “On the Size of the Active Site in Proteases I. Papain,” Biochem Biophys Res Commun 27:157-162 (1967), which is hereby incorporated by reference in its entirety)), specifically leucine and phenylalanine (Rawlings et al., MEROPS: The Peptidase Database (Cambridge CB10 1SA, UK) and Rawlings et al., “MEROPS: The Peptidase Database,” Nucleic Acids Res 36:D320-325 (2008), which are hereby incorporated by reference in their entirety). Importantly, and in keeping with this characteristic, the sequence surrounding the primary H3 cleavage site (A21/T22) mapped from ES cells by both MS and Edman degradation (FIG. 2C, solid line) includes a leucine at the P2 position (L20). To verify the significance of this leucine in the H3 sequence and support the identification of Cathepsin L in fraction 23, it was mutated (L20V, L20E & L20W), along with neighboring residues and the recombinant proteins were tested in the H3 cleavage assay (FIG. 4C). Although the more conservative L20V mutant showed little change in H3 cleavage, L20E and L20W mutants completely abolished H3 cleavage as assayed by both the HIS and H3.cs1 antibodies (FIG. 4C, top two panels). Moreover, the loss of H3 cleavage upon mutation of L20, compared to little or no loss of signal upon mutation of neighboring residues (K18 or A21), suggests that L20 is particularly important to the enzymatic activity in fraction 23. Together, this mutational analysis also supports the identification of Cathepsin L as the enzyme responsible for the histone H3 cleavage characterized in the above in vitro assays.

Interestingly, mutating K23 to either S or Q also had a significant effect on the H3 cleavage activity. Although no difference was seen in H3 cleavage by assaying with HIS, these mutations greatly diminished the cleavage site recognized by the H3.cs1 antibody, suggesting a possible role for this residue in regulating the precision of H3 proteolysis by Cathepsin L. It is interesting to note that none of the N-terminal peptides identified by MS were acetylated at K23 while C-terminal peptides acetylated at K23 were consistently detected (FIG. 9). Together, these data suggest the intriguing possibility that the acetylation of K23 may serve to inhibit cleavage at certain sites.

### Example 15

### Cathepsin L is Associated with Chromatin In Vivo

Although there is previous evidence that Cathepsin L and its activity exists in the cell nucleus of MEFs (Goulet et al., “A Cathepsin L Isoform That is Devoid of a Signal Peptide Localizes to the Nucleus in S Phase and Processes the CDP/Cux Transcription Factor,” Mol Cell 14:207-219 (2004), which is hereby incorporated by reference in its entirety), the nuclear localization in ESCs was documented using a biochemical approach: chromatin was isolated and then solubilized by micrococcal nuclease digestion as described above. As shown by probing with Cathepsin L antibody, Cathepsin L was indeed associated with chromatin fragments released by nuclease digestion (FIG. 4D). Importantly, chromatin association only begins to appear upon differentiation and is not apparent in undifferentiated ES cells. It was also noted that the mature form of Cathepsin L (marked by asterisk) was enriched over the full length (hash) or intermediate (dot) forms.

### Example 16

### rCathepsin L Reproduces the In Vivo Histone H3Cleavage Pattern

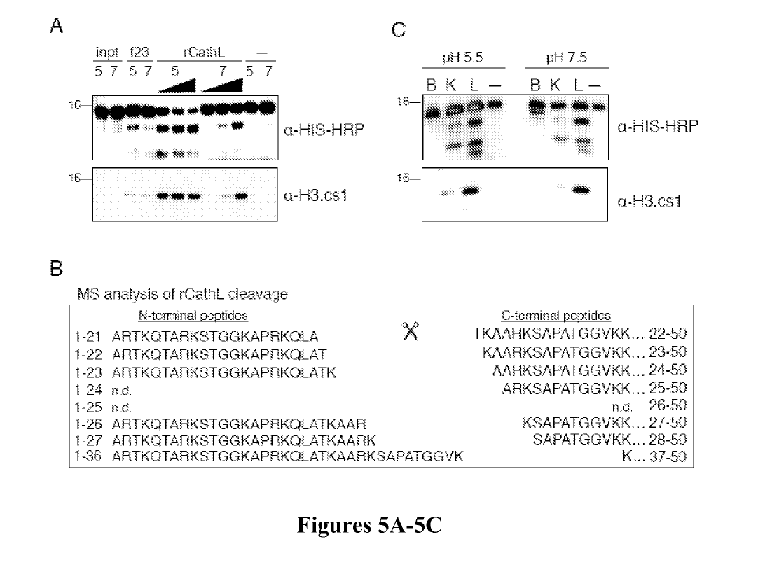

To further validate the identification of Cathepsin L as an H3 protease, recombinant mouse Cathepsin L enzyme was tested in the H3 cleavage assay (FIG. 5). Since Cathepsin L displays optimal activity at the more acidic pH of the lysosome (i.e. ˜pH 5) (Barrett et al., “L-trans-Epoxysuccinyl-leucylamido(4-guanidino)butane (E-64) and its Analogues as Inhibitors of Cysteine Proteinases Including Cathepsins B, H and L,” Biochem J201:189-198 (1982), which is hereby incorporated by reference in its entirety), and since it was suspected that Cathepsin L was acting within the nucleus at a higher pH (˜pH 7-8), the recombinant enzyme was tested in both pH environments. Importantly, recombinant mouse Cathepsin L was able to cleave recombinant histone H3 at both pH 5.5 and pH 7.4. Moreover, the recombinant enzyme not only cleaved H3, but also created the specific epitope recognized by the cleavage site-specific H3.cs1 antibody (FIG. 5A, lower panel). To further characterize the sites of proteolytic cleavage, the reaction products of rCathepsin L and rH3-HIS were analyzed by MS. Importantly, MS analysis revealed that the six sites of cleavage mapped in the endogenous H3 samples (FIG. 2) were also produced by rCathepsin L in vitro (FIG. 5B). Also observed in both reactions were five of the six complementary N-terminal peptides generated by rCathepsin L cleavage of H3 (left), and their abundances correlated, as expected, with that of their C-terminal counterparts. Furthermore, the most abundant site of cleavage in vitro was between A21 and T22, the same site that was shown to be most abundant in the MS analysis of endogenous histone H3. In addition, the relative abundances of the in vivo H3 cleavage sites and those produced by Cathepsin L in vitro are highly correlative. One exception is cleavage between residues K27 and S28, which is more abundant in the in vitro assay compared to that mapped from in vivo samples (the latter shows a preference for cleavage between residues R26/K27 over K27/S28). It was speculated that the presence of histone modifications in vivo play a role in regulating the preferred cleavage site of Cathepsin L, particularly at sites surrounding H3K27, which was shown to be preferentially methylated on cleaved H3 by both immunoblot and MS analysis (FIG. 1A and FIG. 9). Taken together, the above data support the conclusion that Cathepsin L is capable of generating all of the histone H3.2 fragments observed at day three following induction of ESC differentiation by retinoic acid.

Other cathepsin family members were then tested in the H3 cleavage assay. rCathepsin B, one of the most abundant lysosomal proteases, and rCathepsin K, which is reported to have a significant preference for leucine in the P2 position of its substrates much like Cathepsin L, were chosen to be tested first (Cathepsin S also shares this preference but was not found to have significant expression in our ESC model). Following pre-activation the recombinant enzymes, Cathepsins B, K, and L were incubated with rH3-HIS at both pH 5.5. and pH 7.5, and the reactions were then analyzed by immunoblotting with both HIS and H3.cs1 antibodies. As shown in FIG. 5C, pre-activated Cathepsin L cleaves rH3-HIS robustly and produces a pattern that is similar to that observed in vivo, while, in contrast, Cathepsin B cleaves rH3 with a distinct pattern from Cathepsins K or L and does not significantly produce the H3.cs1 epitope. Cathepsin K produces a similar pattern to Cathepsin L under these assay conditions, although they appear to differ in their preferences for specific sites (e.g., the cleavage site recognized by the H3cs.1 antibody is less abundant in the Cathepsin K reactions).

### Example 17

### RNAi-mediated and Chemical Inhibition of Cathepsin L Inhibits H3 Cleavage In Vivo

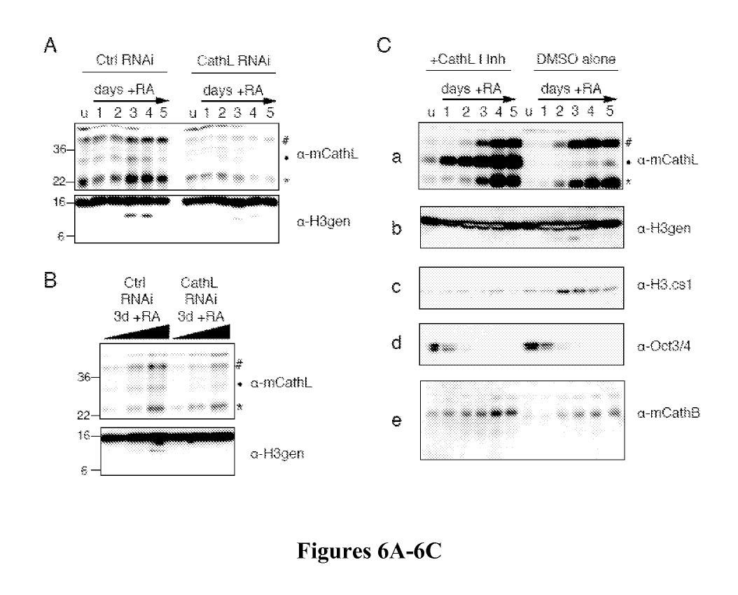

Stable cells lines constitutively expressing short RNAi hairpins to a control gene (a human gene that does not share sequence homology to the mouse genome) and the Cathepsin L gene (Ctsl) were differentiated with RA, as usual, and cells were harvested at various time points. In parallel, samples taken at day 3 post-induction with RA were also evaluated for knockdown efficiency and H3 cleavage by titration of sample (FIG. 6B). As shown in FIGS. 6A and 6B, knockdown of Cathepsin L (shown by immunoblotting with Cathepsin L antibody, upper panels) led to reproducible decrease in H3 cleavage as detected by the H3C-terminal antibody (lower panels).

To further assess whether Cathepsin L causes histone H3 cleavage in vivo, a commercially available, cell-permeable Cathepsin L specific inhibitor, Cathepsin L Inhibitor I, was used Inhibition of Cathepsin L using a chemical inhibitor allowed the effect of Cathepsin L inhibition to be assessed within a single, wild-type cell line rather than comparing individual cell lines created by drug selection. Undifferentiated ESCs were treated with or without inhibitor for 24 hours prior to plating for differentiation. Cells were then differentiated with RA while either Cathepsin L Inhibitor I or DMSO alone was maintained in the media. As shown in FIG. 6C, cells treated with inhibitor showed significant failure to fully process Cathepsin L from its intermediate form (marked by dot) into its mature form (asterisk), demonstrating that its activity had been inhibited (FIG. 6C, panel a, left). In contrast, no significant accumulation of the intermediate form was seen in the DMSO alone treated cells (FIG. 6C, panel a, right). Notably, histone H3 cleavage also decreased significantly in the cells treated with the inhibitor, but not in cells treated with DMSO alone, as shown by probing immunoblots with both H3-general and H3.cs1 antibodies (FIG. 6C, panels b and c). Interestingly, all cleavage detected by the H3-general antibody was abolished in the cells treated with inhibitor, suggesting that Cathepsin L is responsible for all sites of cleavage (supported by MS data in FIG. 5).

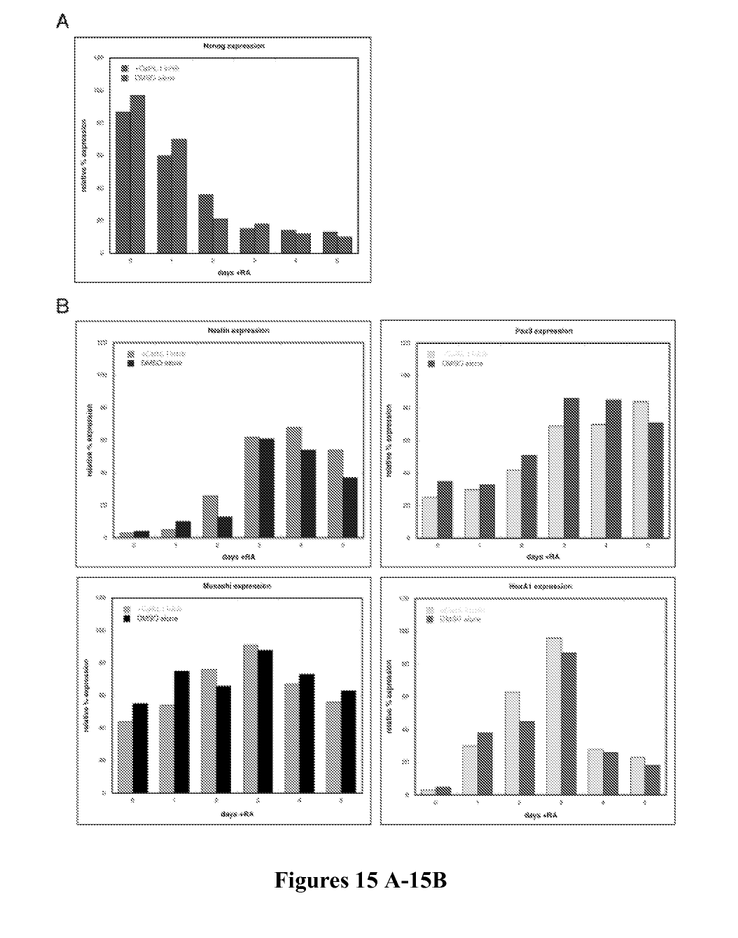

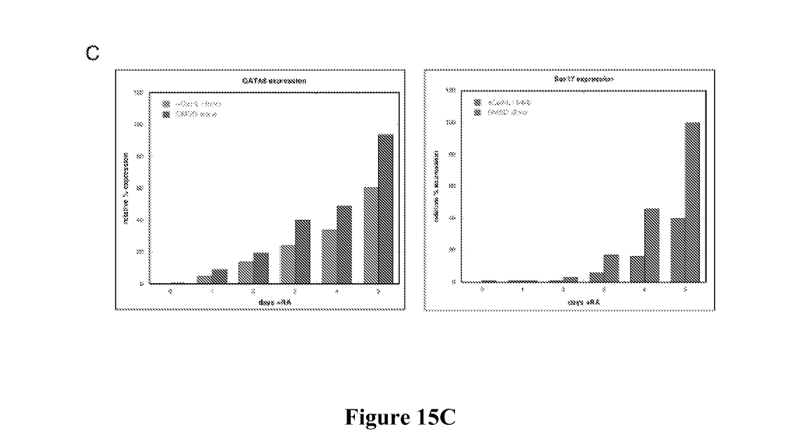

Importantly, neither Oct-3/4 nor Cathepsin B levels (FIG. 6C, panels d and e) appeared to change upon addition of the inhibitor. Since Oct-3/4 is a marker of pluripotency that is normally lost rapidly upon differentiation with RA (FIG. 1C), the fact that this pattern is unchanged in the presence of the Cathepsin L Inhibitor I indicates that the inhibitor does not alter the pluripotency or differentiation capacity of ESCs prior to the time point at which H3 cleavage is observed. This conclusion is supported by quantitative PCR (Q-PCR) data for the expression of the pluripotency marker Nanog (FIG. 15), which also remains unaffected by Cathepsin L inhibition. The consistency of Cathepsin B levels between those cells treated with Cathepsin L Inhibitor I and control cells suggests that the inhibitor is indeed specific for Cathepsin L. Markers of cell lineage were also analyzed by Q-PCR, and these data suggest some changes in neural/ectodermal expression patterns and levels of endodermal marker expression between inhibitor treated and control cells (FIG. 15).

### Example 18

### Histone Tail Modifications Can Modulate Cathepsin L Activity and Its Downstream Effects

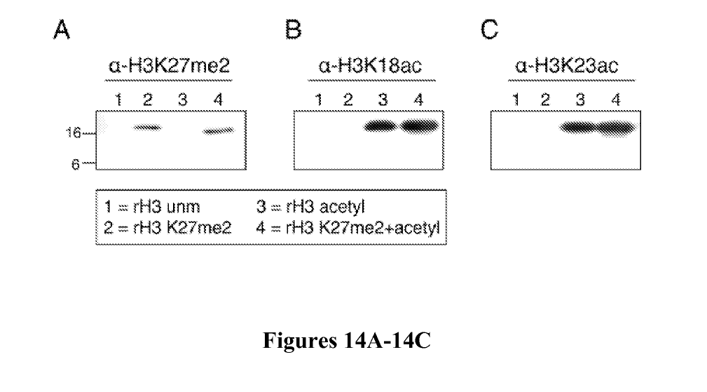

It was next asked whether known covalent modifications on the histone tail affect the cleavage activity of Cathepsin L. As depicted in FIG. 2C, several amino acids near the sites of Cathepsin L cleavage are known to be modified by either acetylation (triangles) or methylation (circles). To test the potential effects of these modifications on the cleavage activity of Cathepsin L, the H3 cleavage assay described in FIG. 3 was employed. Four different recombinant H3 substrates were prepared as follows: unmodified rH3 (1), rH3 dimethylated (me2) specifically at K27 (2), rH3 “pan-acetylated” by treatment with acetic anhydride (3), and rH3 with both K27me2 and pan-acetylation (4). These substrates were shown to have the specific modifications of interest by immunoblot (FIG. 14) and were verified by MS (>90%). As shown in FIG. 7A, the acetylation of lysine residues greatly reduced cleavage of H3 by rCathepsin L at both pH 7.5 and 5.5 (compare substrate 1 to 3). In contrast, K27me2 increases H3 cleavage (compare substrate 1 to 2; greater depletion of full-length rH3+K27me2 suggesting increased cleavage activity at pH 5.5).

In order to test the effect of these modifications more quantitatively and specifically, a set of five peptides with identical backbone sequences flanking the H3 cleavage site (H3 15-31) were synthesized and then modified as follows: unmodified, K18 acetyl, K23 acetyl, K18ac+K23ac, and K27me2. An ELISA based assay using the H3cs.1 antibody described previously (characterized in FIG. 11) was used to quantitatively measure the H3cs.1 cleavage activity of rCathepsin L on these peptides. As shown in FIG. 7B, clear differences in rCathepsin L activity were observed at both pH 7.5 and pH 5.5. As suggested by the rH3 cleavage assay described above, K27me2 (magenta) strongly increases the ability of Cathepsin L to cleave at H3cs.1 as compared to the matched unmodified H3 15-31 peptide (red). Interestingly, acetylation at K18 (blue) also increases this activity, suggesting that the acetylation of another lysine or combination of lysines must be responsible for the abrogation of cleavage by acetylation demonstrated above. This data suggest that acetylation at K23ac is at least partly responsible for this effect, as the K23ac peptide shows very little cleavage activity at H3cs.1, both alone (green) and in combination with K18ac (black).

What effect the cleavage of the H3 tail, a proteolytic modification rather than a covalent modification, might have on the binding of effector proteins was examined. Given that K27 methylation is a well-documented binding site for the chromodomain-containing protein Polycomb (Bernstein et al., “Mouse Polycomb Proteins Bind Differentially to Methylated Histone H3 and RNA and are Enriched in Facultative Heterochromatin,” Mol Cell Biol 26:2560-2569 (2006) and Fischle et al., “Molecular Basis for the Discrimination of Repressive Methyl-lysine Marks in Histone H3 by Polycomb and HP1 Chromodomains,” Genes Dev 17:1870-1881 (2003), which are hereby incorporated by reference in their entirety), the effect of H3 cleavage on Pc binding to H3K27 methylation was tested. Using peptides that mimicked either the “non-cleaved” H3 tail (H3 18-37) or the “cleaved” H3 tail (H3 22-37), the effects of H3 cleavage on the ability for the chromodomain of Pc (mouse CBX7) to bind to K27 methylation (FIG. 7C) was assayed. The PHD-finger of the known H3K4 methyl binding protein BPTF was used as a positive control and to demonstrate specificity (FIG. 7C, lower panel). As shown in FIG. 7C, upper panel, and quantified in FIG. 7D, H3 cleavage greatly diminished CBX7 binding to methylated H3K27, suggesting that proteolytic modification introduced by Cathepsin L could lead to significant downstream effects.

### Discussion of Examples 1-18

A rapidly growing literature demonstrates that cells undergo dramatic developmentally-regulated changes in aspects including gene expression and cellular morphology when transitioning from undifferentiated stem cells to those of specific lineages (Giadrossi et al., “Chromatin Organization and Differentiation in Embryonic Stem Cell Models,” Curr Opin Genet Dev 17:132-138 (2007); Kim et al., “An Extended Transcriptional Network for Pluripotency of Embryonic Stem Cells,” Cell 132:1049-1061 (2008); and Murry et al., “Differentiation of Embryonic Stem Cells to Clinically Relevant Populations: Lessons from Embryonic Development,” Cell 132:661-680 (2008), which are hereby incorporated by reference in their entirety). Despite remarkable progress made in documenting epigenetic signatures such as “bivalent domains” i.e., a H3 tail bearing both H3K4me3 and H3K27me3 marks (Bernstein et al., “A Bivalent Chromatin Structure Marks Key Developmental Genes in Embryonic Stem Cells,” Cell 125:315-326 (2006), which is hereby incorporated by reference in its entirety), little is known as to what mechanisms may function to bring about the above changes at a chromatin level. The data herein suggests that mouse ESCs employ a novel, regulated histone H3 proteolysis mechanism that may serve to alter epigenetic signatures upon differentiation. Other means of actively removing histone methyl marks have been well documented, including enzymatic demethylation (Anand et al., “Structure and Mechanism of Lysine-Specific Demethylase Enzymes,” J Biol Chem 282:35425-35429 (2007) and Shi et al., “Histone Demethylation Mediated by the Nuclear Amine Oxidase Homolog LSD1,” Cell 119:941-953 (2004), which are hereby incorporated by reference in their entirety) and selective histone variant replacement (Ahmad et al., “The Histone Variant H3.3 Marks Active Chromatin by Replication-Independent Nucleosome Assembly,” Mol Cell 9:1191-1200 (2002), which is hereby incorporated by reference in its entirety). However, considerably less evidence documenting endogenous histone proteolysis has been reported (Allis et al., “Proteolytic Processing of Histone H3 in Chromatin: A Physiologically Regulated Event in Tetrahymena Micronuclei,” Cell 20:55-64 (1980) and Falk et al., “Foot-and-Mouth Disease Virus Protease 3C Induces Specific Proteolytic Cleavage of Host Cell Histone H3,” J Virol 64:748-756 (1990), which are hereby incorporated by reference in their entirety) and in scarcely few cases have the responsible proteases been identified. The identification and characterization of developmentally-regulated H3 cleavage by Cathepsin L during ESC differentiation is an important step in understanding limited nuclear histone proteolysis as a potential mode of transcriptional regulation.

The finding that Cathepsin L is responsible for the controlled histone H3 cleavage activity in mouse ES cells was not expected, in part as this enzyme was originally described as a lysosomal (hence “L”) protease (Barrett et al., “L-trans-Epoxysuccinyl-leucylamido(4-guanidino)butane (E-64) and its Analogues as Inhibitors of Cysteine Proteinases Including Cathepsins B, H and L,” Biochem J 201:189-198 (1982), which is hereby incorporated by reference in its entirety).

However, several lines of evidence suggest that nuclear functions of Cathepsin L exists. First, Cathepsin L has been shown to localize to nuclei in a cell-cycle dependent manner where it plays a role in the proteolytic processing of transcription factor CDP/Cux (Goulet et al., “A Cathepsin L Isoform That is Devoid of a Signal Peptide Localizes to the Nucleus in S Phase and Processes the CDP/Cux Transcription Factor,” Mol Cell 14:207-219 (2004), which is hereby incorporated by reference in its entirety). Biochemical studies have also shown that pro-Cathepsin L is localized to the nucleus in ras-transformed mouse fibroblasts (Hiwasa et al., “Nuclear Localization of Procathepsin L/MEP in Ras-Transformed Mouse Fibroblasts,” Cancer Lett 99:87-91 (1996), which is hereby incorporated by reference in its entirety). Second, evidence of endogenous, nuclear serpin inhibitors with the ability to inhibit Cathepsin L activity, e.g., MENT (Bulynko et al., “Cathepsin L Stabilizes the Histone Modification Landscape on the Y Chromosome and Pericentromeric Heterochromatin,” Mol Cell Biol 26:4172-4184 (2006), which is hereby incorporated by reference in its entirety) and Cystatin B (Riccio et al., “Nuclear Localization of Cystatin B, the Cathepsin Inhibitor Implicated in Myoclonus Epilepsy (EPM1),” Exp Cell Res 262:84-94 (2001), which is hereby incorporated by reference in its entirety), further supports the notion that Cathepsin L, and potentially other cysteine proteases, play important but poorly understood roles in regulating nuclear proteolysis. With the exception of transcription factor CDP/Cux, nuclear substrates of Cathepsin L have not been identified to date and histones have not yet been identified as physiologically-relevant substrates of this class of proteases in mammalian cells. Interestingly, however, recent studies in sea urchin have suggested that a Cathepsin L-like cysteine protease may be responsible for the degradation of sperm histones during a key chromatin remodeling event after fertilization (Morin et al., “Cathepsin L Inhibitor I Blocks Mitotic Chromosomes Decondensation During Cleavage Cell Cycles of Sea Urchin Embryos,” J Cell Physiol. (2008), which is hereby incorporated by reference in its entirety).

Several lines of evidence suggest that histone H3 is a substrate for Cathepsin L, at least at some genomic loci and in certain developmental contexts. First, the histone H3 sequence, specifically the P2 position to the first mapped endogenous cleavage site (H3.cs1, see FIG. 2C), agrees well with the documented sequence preference of Cathepsin L (Rawlings et al., “MEROPS: The Peptidase Database,” Nucleic Acids Res 36:D320-325 (2008), which is hereby incorporated by reference in its entirety). Second, chemical inhibitors to cysteine proteases, those specific to Cathepsin L, and RNAi mediated knockdown of the Ctsl gene all attenuate histone H3 cleavage both in vitro and in vivo. Significant inhibition of H3 cleavage activity was observed with the addition of moderately high concentrations of EDTA or EGTA. Although Cathepsin L is not a metalloprotease, its inhibition by these chelators has been documented previously (Hara et al., “Effect of Proteinase Inhibitors on Intracellular Processing of Cathepsin B, H and L in Rat Macrophages,” FEBS Lett 231:229-231 (1988), which is hereby incorporated by reference in its entirety). Third, recombinant Cathepsin L cleaves H3 in vitro with a remarkably similar pattern to that mapped in vivo. Moreover, differences in the relative abundances of the sites produced may be explained by the fact that Cathepsin L activity is modulated by the presence of specific histone modifications. Thus, the view that controlled H3 proteolysis is limited to a small fraction of developmentally-regulated loci that may contain unique epigenetic signatures is favored

The finding that Cathepsin L is an H3 protease is interesting when considering the phenotype common to the Cathepsin L knockout mouse (Nakagawa et al., “Cathepsin L: Critical Role in Ii Degradation and CD4 T Cell Selection in the Thymus,” Science 280:450-453 (1998), which is hereby incorporated by reference in its entirety) and the furless mouse, which has been shown to have a spontaneous mutation in the Cathepsin L gene (Roth et al., “Cathepsin L Deficiency as Molecular Defect of Furless: Hyperproliferation of Keratinocytes and Perturbation of Hair Follicle Cycling,” FASEB J 14:2075-2086 (2000), which is hereby incorporated by reference in its entirety). These mice exhibit periodic hair loss due to the improper cycling and morphogenesis of their hair follicles, suggesting a defect in stem cell renewal and/or differentiation. Cathepsin L knockout mice are viable and fertile, however, indicating that its functions are nonessential and/or redundant. Interestingly, although Cathepsin B mice are also viable and show no obvious phenotype, Cathepsin L/Cathepsin B double knockout mice exhibit severe brain atrophy and die two to four weeks after birth (Felbor et al., “Neuronal Loss and Brain Atrophy in Mice Lacking Cathepsins B and L,” Proc Natl Acad Sci USA 99:7883-7888 (2002), which is hereby incorporated by reference in its entirety). The severity and neural selectivity of this phenotype suggests that these two enzymes overlap in their specific functions. Although significant inhibition of Cathepsin B was not observed upon in vivo chemical inhibition of Cathepsin L, nor was the same pattern of H3 cleavage with recombinant Cathepsin B compared to Cathepsin L observed in vitro, the possibility of redundancy in H3 cleavage function between these or other related enzymes at other stages or lineages of differentiation cannot be excluded.

Limited proteolysis of nuclear proteins is an important means of regulating transcription and other cell processes (Goulet et al., “Complete and Limited Proteolysis in Cell Cycle Progression,” Cell Cycle 3:986-989 (2004) and Vogel et al., “Site-Specific Proteolysis of the Transcriptional Coactivator HCF-1 Can Regulate its Interaction With Protein Cofactors,” Proc Natl Acad Sci USA 103:6817-6822 (2006), which are hereby incorporated by reference in their entirety). Here it was shown that limited proteolysis of histone H3 by Cathepsin L occurs during differentiation of ESCs and may be regulated both positively and negatively by other covalent modifications on the H3 tail itself. The data herein support other studies that demonstrate the nuclear localization of Cathepsin L and provide the first indication that cellular histones, H3 in particular, are key substrates of this family of proteases.

The current findings require a revaluation as to the function of this family of enzymes in transcriptional and epigenetic regulation (Chapman, H. A., “Cathepsins as Transcriptional Activators?” Dev Cell 6:610-611 (2004), which is hereby incorporated by reference in its entirety). Many important questions remain. First, how is the protease cleavage activity regulated? The data herein demonstrate that covalent modifications (such as acetylation and/or methylation of nearby lysines) serve to regulate, positively and negatively, the H3 proteolytic processing event. Here it is noted that proteolytic processing of H3 in the ciliate model occurs selectively in a hypoacetylated, transcriptionally silent (micronuclear) genome, while processing of H3 fails to occur in hyperacetylated, transcriptionally active macronuclei (Allis et al., “Proteolytic Processing of Histone H3 in Chromatin: A Physiologically Regulated Event in Tetrahymena Micronuclei,” Cell 20:55-64 (1980), which is hereby incorporated by reference in its entirety). These data support the hypothesis that other chromatin modifying enzyme complexes, such as HATs or HDACs, play a role in regulating histone proteolysis. Along these same lines, it is feasible that histone mono-ubiquitylation might lead to limited histone “clipping,” as compared to the wholesale protein degradation that is signaled by poly-ubiquitinylation.

Second, by what mechanism is the cleaved H3 replaced, and is DNA replication and chromatin assembly required? The current findings that the histone variant H3.2 is preferably cleaved as compared to H3.3 suggests that S-phase/replication-coupled replacement may be involved (Loyola et al., “Marking Histone H3 Variants: How, When and Why?,” Trends Biochem Sci 32:425-433 (2007), which is hereby incorporated by reference in its entirety). Along this line, it was noted that proteolytic processing of transcription factor CDP/Cux by Cathepsin L occurs during the G1/S-phase transition and is coupled to cell cycle progression (Goulet et al., “A Cathepsin L Isoform That is Devoid of a Signal Peptide Localizes to the Nucleus in S Phase and Processes the CDP/Cux Transcription Factor,” Mol Cell 14:207-219 (2004), which is hereby incorporated by reference in its entirety). Whether proteolytic processing of H3 is cell cycle dependent remains unclear, although it is interesting to note that the present studies were done in ESCs, which cycle rapidly and spend a high percentage of time in S-phase as measured by flow cytometry.

Finally, with other mechanisms of histone demethylation well documented (i.e., enzymatic demethylation, variant replacement, etc.), it is possible histone proteolysis serves other purposes that are not appreciated, such as the generation of new N-termini. Here, it was noted that the engagement of end-binding-effector modules may be facilitated by removing the steric hindrance of fragments more N-terminal to their binding sites. (Ruthenburg et al., “Methylation of Lysine 4 on Histone H3: Intricacy of Writing and Reading a Single Epigenetic Mark,” Mol Cell 25:15-30 (2007), which is hereby incorporated by reference in its entirety). Alternatively, histone cleavage may remove critical recognition elements and thereby block the binding of downstream effectors (See FIG. 7D).

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1.-81. (canceled)

82. A method comprising:
administering to a cell an agent that modulates histone proteolysis at a motif comprising KQLATK (SEQ ID NO:4) of the histone.

83. The method according to claim 82, wherein the agent modulates histone proteolysis of histone-3.

84. The method according to claim 82, wherein the agent inhibits cathepsin.

85. The method according to claim 84, wherein the cathepsin inhibitor is selected from the group consisting of a nucleic acid, a peptide, and a small molecule.

86. The method according to claim 85, wherein the cathepsin inhibitor is a siRNA molecule directed to cathepsin L and having a nucleotide sequence of SEQ ID NO:7 or SEQ ID NO:8.

87. The method according to claim 85, wherein the cathepsin inhibitor is selected from the group consisting of Z-Phe-Phe-CH2F, Z-Phe-Tyr-CHO, Z-LLY-CHN2, Ac-LLnL-CHO-ALLN, aprotinin, leupeptin, N-morpholineurea-phenylalanyl-homophenylalanylfluoromethyl ketone, Z—FF-FMK, Z-LL-FMK, Z-Phe-Tyr(t-Bu)-diazomethylketone, LLLTR-NH2, RKLLW-NH2, LFLTR-NH2, RKLWL-NH2, RKLWD-NH2, an alpha-ketoamide derivative, an acylaminoaldehyde derivative, and a thiocarbazate derivative.

88. The method according to claim 82, wherein the agent is a recombinant cathepsin protein or proteolytic active cathepsin polypeptide.

89. The method according to claim 82, wherein the agent is a nucleic acid molecule encoding a recombinant cathepsin protein or proteolytic active cathepsin polypeptide.

90. The method according to claim 82, wherein the agent is a recombinant cathepsin L protein or proteolytic active cathepsin L polypeptide.

91. The method according to claim 82, wherein the agent modulates amino acid acetylation.

92. The method according to claim 91, wherein the agent is a histone acetyltransferase inhibitor.

93. The method according to claim 92, wherein the histone acetyltransferase inhibitor is selected from the group consisting of a coenzyme A conjugate, a polyisoprenylated benzophenone, a curcumin derivative, a quinoline derivative, and an isothiazolone.

94. The method according to claim 91, wherein said agent is a histone deacetylase inhibitor.

95. The method according to claim 94, wherein the histone deacetylase inhibitor is selected from the group consisting of nucleoplasmin, chamydocin, Cyl-2, cyclic(eta-oxo-alpha-aminooxiraneoctanoylphenylalanylleucyl-2-piperidinecarbonyl (WF-3161), depudecin, radicocol, oxamfiatin, apidicin, suberoxylanilide hydroxamic acid, 2-amino-8-oxo-9,10-epoxy-decanoic acid, butyrate, trapoxin analogs, trichostatin A, valproic acid and its derivatives, carbamic acid compounds comprising sulfonamide linkages, compounds having a zinc-binding moiety, cyclic tetrapeptide derivatives m-carboxycinnamic acid bis-hydroxamie, FK228, M344, and 3-(4-aroyl-2-pyrrolyl)-N-hydroxy-2-propenamide.

96. A method of suppressing stem cell differentiation, said method comprising:
administering to a population of stem cells an agent that inhibits histone proteolysis at a motif comprising KQLATK (SEQ ID NO:4) of the histone under conditions effective to regulate stem cell differentiation, wherein the agent is a cathepsin inhibitor.

97. A method of decreasing gene transcription in a cell, said method comprising:
administering to a population of cells an agent that inhibits histone proteolysis at a motif comprising KQLATK (SEQ ID NO:4) of the histone under conditions effective to modulate gene transcription in the cell, wherein the agent is a cathepsin inhibitor.

98. A method of identifying candidate compounds useful for modulating histone proteolysis comprising:
providing the candidate compound;
providing a population of differentiating stem cells;
contacting the candidate compound with the population of differentiating stem cells under conditions effective for the candidate compound to modulate histone proteolysis;
detecting the presence or absence of a histone cleavage product in the population of differentiated stem cells; and
identifying a compound useful for modulating histone proteolysis based on said detecting.

99. A method of treating a subject having cancer, said method comprising:
selecting a patient based on his/her propensity to undergo histone proteolysis and
administering an agent that modulates histone proteolysis to the subject under conditions effective to treat cancer.

100. A method comprising:
administering to a cell an agent that inhibits histone proteolysis in the cell, wherein the agent is a cathepsin inhibitor selected from the group consisting of a nucleic acid, a peptide, and a small molecule cathepsin inhibitor.

101. A method comprising:
administering to a cell an agent that induces histone proteolysis in the cell, wherein the agent is a recombinant cathepsin protein, a proteolytic active cathepsin polypeptide, or a nucleic acid molecule encoding the cathepsin protein or proteolytic active cathepsin polypeptide.
